(19) United States
(12) Patent Application Publication
(10) Pub. No.: US 2021/0123021 A1
(43) Pub. Date: Apr. 29, 2021

(54) NEUTROPHIL SUBTYPES

(71) Applicant: Agency for Science, Technology and Research, Singapore (SG)

(72) Inventors: Lai Guan NG, Singapore (SG); Maximilien EVRARD, Singapore (SG); Immanuel Weng Han KWOK, Singapore (SG)

(21) Appl. No.: 16/965,197

(22) PCT Filed: Jan. 25, 2019
(86) PCT No.: PCT/SG2019/050040

§ 371 Date: Jul. 27, 2020

(30) Foreign Application Priority Data
Jan. 26, 2018 (SG) 10201800714R

(51) Int. Cl. C12N 5/0787; C07K 14/705; G01N 33/50

(52) U.S. Cl. CPC C12N 5/0642; C07K 14/70596; G01N 2496/05; G01N 2800/7095; G01N 2800/7028; G01N 33/5091

(57) ABSTRACT

Disclosed is a method of characterising and/or separating neutrophils, the method comprises characterising and/or separating the neutrophils into a first population comprising proliferative neutrophils and a second population comprising mature neutrophils, according to the expression of CD101 on the neutrophils. Also disclosed are compositions comprising proliferative neutrophils that are CD10⁻CD101⁻, and methods of treatment, diagnostic or prognostic using neutrophils thereof, as well as kits for characterising and/or separating proliferative neutrophils based on the expression of CD101 or CD10. In a preferred embodiment, the population of neutrophils may be characterised as proliferative neutrophils if CD10⁻CD101⁻, as immature neutrophils if CD10⁻CD101⁺ and as mature neutrophils if CD10⁺CD101⁺.

### TECHNICAL FIELD OF THE INVENTION

The present disclosure relates to neutrophils, methods of categorizing neutrophils into neutrophil subtypes and separating and/or isolating/enriching the same. The present disclosure also relates to therapeutic, diagnostic and prognostic methods or kits related to neutrophil subtypes.

### BACKGROUND OF THE INVENTION

Neutrophils are indispensable cells of the early innate immune response against pathogens. Any defect in neutrophil generation can lead to life threatening conditions, and hence their development needs to be tightly regulated. Due to their short half-life, neutrophils require a constant replenishment from proliferative bone marrow (BM) precursors. While it is well established that neutrophils are derived from granulocyte-macrophage progenitor (GMP), the differentiation pathways from GMP to functional mature neutrophils are poorly defined.

### SUMMARY OF THE INVENTION

The present invention seeks to provide a method of categorizing/characterising neutrophils into neutrophil subtypes and separating and/or isolating/enriching the same. The present invention also seeks to provide kits, and therapeutic, diagnostic and prognostic methods related to neutrophil subtypes.

According to one aspect of the present invention, there is provided a method of characterising and/or separating neutrophils, the method comprises characterising and/or separating the neutrophils into a first population comprising proliferative neutrophils and a second population comprising mature neutrophils, according to the expression of CD101 on the neutrophils.

In some examples, the first population expresses CD101⁻ and the second population expresses CD101⁺.

In some examples, when the neutrophils are human neutrophils, the method may further comprise characterising and/or separating the neutrophils according to the expression of CD10 on the neutrophils, wherein the first population comprising proliferative neutrophils are CD10⁻CD101⁻ and the second population comprising mature neutrophils are CD10⁺CD101⁺, optionally the second neutrophils population further comprises immature neutrophils that are CD10⁻CD101⁺.

In some examples, the method may further comprise characterising and/or separating the neutrophils according to the expression of one or more biomarkers selected from the group consisting of CD49d, CD16, CXCR2, CD34, CD66, CD15, CD71, and CD11b.

In some examples, the proliferative neutrophils may comprise pro-neutrophils and pre-neutrophils.

In some examples, the pro-neutrophils may be CD101⁻CD10⁻CD16⁻ CD34⁻CD66b⁺CD15⁺CD71⁺CD49d⁺CD11b⁻CXCR2⁻, the pre-neutrophils may be CD101⁻CD10⁻CD16⁻CD34⁻CD66b⁺CD15⁺CD71⁺CD49d⁺CD11b⁺CXCR2⁻, the immature neutrophils may be CD101⁺CD10⁻CD16⁻CD34⁻CD66b⁺CD15⁺CD71⁻ CD49d^{lo}CD11b⁺CXCR2⁻, and the mature neutrophils may be CD101⁺CD10⁺CD16⁺CD34⁻CD66b⁺CD15⁺CD71⁻CD49d^{lo}CD11b⁺CXCR2⁺.

In some examples, when the neutrophils are murine neutrophils, the method may further comprise characterising and/or separating the neutrophils according to the expression of cKit on the neutrophils, wherein the first population comprising proliferative neutrophils may be one of cKit^{hi}CD101⁻, cKit^{int}CD101⁻, or cKit^{lo}CD101⁻ and the second population comprises mature neutrophils that may be cKit-CD101⁺. In some examples, the first neutrophils population may further comprise immature neutrophils that are cKit^{lo}CD101⁺.

In some examples, the method may further comprise characterising and/or separating the neutrophils according to the expression of one or more biomarkers selected from the group consisting of CD101, cKit, Ly6C, CD106, SiglecF, CD115, CD205, CD11b, Gr1, and CXCR4.

In some examples, the proliferative neutrophils may comprise pro-neutrophils and pre-neutrophils.

In some examples, pro-neutrophils may be CD101⁻ cKit^{Hi}Ly6C⁺CD106⁺SiglecF⁻CD115⁻CD205⁻CD11b^{Lo}Gr1^{Lo}CXCR4^{Hi}, the pre-neutrophils may be CD101⁻cKit^{lo}Ly6C⁺CD106⁺⁺SiglecF⁻CD115⁻CD205⁺CD11b^{Hi}Gr1^{Hi} CXCR4^{Hi} or CD101⁻cKit^{int}Ly6C⁺CD106⁺⁺SiglecF⁻CD115⁻CD205⁺CD11b^{Hi}Gr1^{Hi} CXCR4^{Hi}, the immature neutrophils may be CD101⁻ cKit^{int}Ly6C⁺CD106⁺SiglecF⁻CD115⁻ CD205⁺CD11b^{Hi}Gr1^{Hi}CXCR4^{Lo} or CD101⁻cKit^{lo}Ly6C⁺CD106⁺SiglecF⁻CD115⁻ CD205⁺CD11b^{Hi}Gr1^{Hi}CXCR4^{Lo} and the mature neutrophils may be CD10⁺cKit⁻ Ly6C⁺CD106^{lo}SiglecF⁻CD115⁻CD205⁺CD11b^{Hi}Gr1^{Hi}CXCR4^{Lo}.

According to another aspect of the present invention, there is provided a kit for separating neutrophils. In some examples, the kit may comprise an agent for detecting the expression of CD101 on the neutrophils; and/or a separator for separating a first population comprising proliferative neutrophils and a second population comprising mature neutrophils according to the expression of CD101 on the neutrophils.

In some examples, the first population may express CD101⁻ and the second population may express CD101⁺.

In some examples, the kit may be for separating human neutrophils and the kit may further comprises an agent for detecting the expression of CD10 on the human neutrophils, and the separator may be adapted to separate the neutrophils according to the expression of CD10 on the neutrophils, wherein the first population comprising proliferative neutrophils are CD10⁻CD101⁻, and the second population comprising mature neutrophils are CD10⁺CD101⁺, optionally the second population further comprises immature neutrophils that are CD10⁻CD101⁺.

In some examples, the agent for detecting the expression of CD10 is an antibody adapted to target CD10, and/or wherein the agent for detecting the expression of CD101 is an antibody adapted to target CD101.

In some examples, the kit may further comprise an agent for detecting the expression on the neutrophils one or more biomarkers selected from a group consisting of CD49d, CD16, CXCR2, CD34, CD66, CD15, CD71, and CD11b, and wherein the separator is adapted to separate the neutrophils according to the expression of one or more of CD49d, CD16, CXCR2, CD34, CD66, CD15, CD71, and CD11b on the neutrophils.

In some examples, the kit may be for separating murine neutrophils, and wherein the separator may be further adapted to separate the neutrophils according to the expression of CD101 and/or cKit, wherein the first population may comprise proliferative neutrophils that are one of cKit^{hi}CD101⁻, cKit^{int}CD101⁻, or cKit^{lo}CD101⁻ and the second population may comprise mature neutrophils are cKit-CD101⁺, optionally, wherein the first population may further comprise immature neutrophils that are cKit^{lo}CD101⁺.

In some examples, the agent for detecting the expression of CD101 and/or cKit may be an antibody adapted to target CD101 and/or cKit.

In some examples, the kit may further comprise an agent for detecting the expression on the neutrophils of one or more biomarkers such as but is not limited to CD101, cKit, Ly6C, CD106, SiglecF, CD115, CD205, CD11b, Gr1, CXCR4, and the like, and wherein the separator may be adapted to separate the neutrophils according to the expression of one of the biomarkers such as but is not limited to CD101, cKit, Ly6C, CD106, SiglecF, CD115, CD205, CD11b, Gr1, and/or CXCR4 on the neutrophils.

According to another aspect of the present invention, there is provided a method of isolating and/or enriching a desired neutrophil. In some examples, the method may comprise categorizing neutrophils in a sample into a first population comprising proliferative neutrophils and a second population comprising mature neutrophils according to the expression of CD101 on the neutrophils. In some examples, the method may further comprise isolating and/or enriching one or more neutrophil from the first population and/or the second population.

In some examples, the sample may be obtained from a human subject. In such examples, the method may further comprise categorizing the neutrophils according to the expression of CD10 on the neutrophils, wherein the first population comprising proliferative neutrophils are CD10⁻CD101⁻ and the second population comprising mature neutrophils are CD10⁺CD101⁺. In some examples, the second population may further comprise immature neutrophils and are CD10⁻CD101⁺.

In some examples, the method may comprise detecting expression of CD10 and/or CD101 with an agent adapted to target CD10 and/or CD101.

In some examples, the method may comprise isolating one or more neutrophil comprises immobilizing the one or more neutrophil via the agent adapted to target CD10 and/or CD101.

In some examples, the method may further comprise the step of validating the neutrophil in the first and/or second population by detecting the expression of one or more biomarkers selected from a group consisting of CD49d, CD16, CXCR2, CD34, CD66, CD15, CD71, and CD11b.

In some examples, the sample may be obtained from a murine subject. In some examples, the first population may comprise proliferative neutrophils that are CD101⁻, and the second population may comprise mature neutrophils that are CD101⁺. In some examples, the first population may further comprise immature neutrophils that are CD101⁻.

In some examples, the method may comprise detecting expression of CD101 with agents adapted to target CD101. In some examples, the method may comprise isolating one or more desired neutrophil subtypes. In such examples, the method may comprise immobilizing the one or more desired neutrophil subtypes via the agents adapted to target CD101.

In some examples, the method may further comprise the step of validating the desired neutrophil subtype by detecting the expression of one or more biomarkers such as but is not limited to CD101, cKit, Ly6C, CD106, SiglecF, CD115, CD205, CD11b, Gr1, and CXCR4.

In some examples, the method may comprise administering the subject with agents such as but is not limited to Plerixafor, granulocyte-colony stimulating factor (G-CSF) and/or interleukin 3 (IL-3) prior to obtaining the population of cells from the subject.

In some examples, the desired neutrophil subtype may be pro-neutrophils and/or pre-neutrophils.

In some examples, the method may further comprise the step of expanding the pro-neutrophils and/or pre-neutrophils with one or more growth factors selected from a group consisting of interleukin 6 (IL-6), leukaemia inhibitory factor (LIF), stem cell factor (SCF), G-CSF and IL-3.

According to another aspect of the present invention, there is provided a composition comprising proliferative neutrophils. In some examples, the proliferative neutrophils may be CD10⁻CD101⁻.

According to another aspect of the present invention, there is provided a composition comprising a therapeutically effective amount of proliferative neutrophils for use in treatment. In some examples, the proliferative neutrophils may be CD10⁻ CD101⁻.

In some examples, the composition may be for use in the treatment of immunodeficiency related diseases and/or disorders in a patient.

According to another aspect of the present invention, there is provided a composition comprising a therapeutically effective amount of proliferative neutrophils for enhancing the immune system of a subject and/or maintaining an immune response in the subject. In some examples, the proliferative neutrophils may be CD10⁻CD101⁻.

In some examples, the proliferative neutrophils may comprise pro-neutrophils and/or pre-neutrophils.

In some examples, the pro-neutrophils may be CD101⁻CD10⁻CD16⁻ CD34⁻CD66b⁺CD15⁺CD71⁺CD49d⁺CD11b⁻CXCR2⁻ and/or the pre-neutrophils may be CD101⁻CD10⁻CD16⁻CD34⁻CD66b⁺CD15⁺CD71⁺CD49d⁺CD11b⁺CXCR2⁻.

According to another aspect of the present invention, there is provided a use of proliferative neutrophils in the manufacture of a medicament for treating immunodeficiency related diseases and/or disorders in a patient. In some examples, the proliferative neutrophils may be CD10⁻CD101⁻.

According to another aspect of the present invention, there is provided a method of treating immunodeficiency related diseases and/or disorders in a patient, the method comprising administering to a therapeutically effective amount of proliferative neutrophils to a patient. In some examples, the proliferative neutrophils may be CD10⁻CD101⁻.

In some examples, the immunodeficiency related disease and/or disorders may be associated with cancer and/or infection.

In some examples, the patient may be immunocompromised.

In some examples, the method may comprise administering the therapeutically effective amount of proliferative neutrophils to the patient every three (3) to five (5) days.

According to another aspect of the present invention, there is provided a method of enhancing the immune system of a patient. In some examples, the method may comprise the steps of (a) obtaining a population of cells comprising neutrophils; (b) isolating proliferative neutrophils from the population of cells according to CD10 and/or CD101 expression on the neutrophils, wherein the proliferative neutrophils are CD10⁻CD101⁻; and (c) administering a therapeutically effective amount of the proliferative neutrophils to the patient.

In some examples, wherein step (b) may further comprise detecting expression of CD10 and/or CD101 with agents adapted to target CD10 and/or CD101.

In some examples, the method may further comprise the step of expanding the pre-neutrophils prior to step (c).

In some examples, the proliferative neutrophils may be expanded with one or more growth factors selected from a group consisting of interleukin 6 (IL-6), leukaemia inhibitory factor (LIF), stem cell factor (SCF), G-CSF and IL-3.

In some examples, step (a) may comprise obtaining the population of cells comprising neutrophils from the patient. In some examples, the population of cells may be from the bone marrow of the patient and/or from cord blood.

According to another aspect of the present invention, there is provided a method for diagnosing or prognosing a medical condition in a patient. In some examples, the method may comprise the steps of: (a) testing a sample comprising neutrophils obtained from a patient, to detect the expression of CD10 and/or CD101 on the neutrophils; (b) measuring the levels of proliferative neutrophils, immature neutrophils and/or mature neutrophils in the sample, wherein proliferative neutrophils are CD10⁻CD101⁻, immature neutrophils are CD10⁻CD101⁺, and mature neutrophils are CD10⁺CD101⁺; and (c) comparing the levels of the proliferative neutrophils, immature neutrophils and/or mature neutrophils in the sample, to reference levels in a control to determine the absence or presence of the medical condition, or to predict the course of the medical condition.

In some examples, the sample may be a bone marrow sample and/or a spleen sample. In such examples, a level of proliferative neutrophils in the sample higher than the reference level in the control may indicate that the patient has an inflammatory medical condition.

In some examples, the inflammatory medical condition may be associated with an autoimmune disease, sepsis and/or cancer.

In some examples, a level of immature neutrophils in the sample higher than the reference level in the control may indicate that the patient has the medical condition. In some examples, the level of immature neutrophils may correlate with the progression of the medical condition.

In some examples, the sample may be a blood sample or a tumor sample. In some examples, the medical condition may be cancer. In some examples, the cancer may be pancreatic cancer.

According to another aspect of the present invention, there is provided a kit for detecting and/or predicting inflammation in a patient, the kit comprising: (a) an agent for detecting the expression of CD10 on neutrophils and/or an agent for detecting the expression of CD101 on neutrophils to measure the level of proliferative neutrophils in a sample taken from the patient, wherein the proliferative neutrophils are CD10⁻ CD101⁻; and (b) a reference level for comparing the measured level of proliferative neutrophils, wherein a level of proliferative neutrophils in the sample higher than the reference level may indicate that the patient has an inflammatory medical condition.

According to another aspect of the present invention, there is provided a kit for diagnosis and/or prognosing cancer in a patient, the kit comprising: (a) an agent for detecting the expression of CD10 on neutrophils and/or an agent for detecting the expression of CD101 on neutrophils to measure the level of immature neutrophils in a sample taken from the patient, wherein the immature neutrophils are CD10⁻ CD101⁺; and (b) a reference level for comparing the measured level of immature neutrophils, wherein a level of immature neutrophils in the sample higher than the reference level may indicate that the patient has cancer, and/or wherein the level of immature neutrophils may correlate with the progression of cancer.

### BRIEF DESCRIPTION OF THE DRAWINGS

The invention will now be described, by way of example only, with reference to the accompanying drawings as follows:

FIG. 1. (A) Visualized t-SNE map of human CD45+ BM cells based on the expression of 40 different markers by mass cytometry. (B-C) Neutrophils were manually gated as Lin-CD15+CD66b+ and were identified as proliferative (IdU+) and non-proliferative (IdU−). Median expression of markers among IdU+ and IdU− neutrophils were next plotted as heat maps to identify differentially expressed markers between proliferating and non-proliferating neutrophils.

FIG. 2. (A) Gating strategy of human BM neutrophil subsets, which are defined as pre-neutrophils (preNeu), immature neutrophils and mature neutrophils. (B) Median expression of surface markers among neutrophil subsets were next plotted as heat maps (blue: low expression; red: high expression). (C) Based on the expression of CD10 and CD101, Lin-CD15+CD66b+ total neutrophils can be subdivided into preNeu, immature and mature neutrophils.

FIG. 3. (A) preNeu and immature neutrophils are mainly localized in the BM but not in the blood at resting state. (B) Neutrophil subsets display similar proliferation status across tissues.

FIG. 4. Intra-BM transfer of sorted Lyz2-GFP+ preNeu into wild type recipients. Black dots represent transferred cells at day 1 (top row) and day 2 (bottom row) after transfer. Data are representative of one out of five independent mice. Eo-eosinohils, Mo-monocytes.

FIG. 5. Mass cytometry reveals proliferative neutrophils with distinct phenotypic signatures. (A) Schematic diagram of the hierarchical order of hematopoiesis adapted from Manz and Boettcher, 2014. (B) Frequency of proliferating cells among various progenitor and mature leukocyte populations by Fucci-(S-G2-M) (#474) mice in vivo. Results are expressed as mean±SD (n=3) and representative of two independent experiments. Peritoneum LPM—peritoneum large peritoneal macrophage; Spleen RPM—spleen red pulp macrophage. (C) Visualized t-SNE maps of IdU⁺ (proliferative) and IdU⁻ (non-proliferative) cells from mouse CD45⁺ BM cells based on the expression of 40 different parameters. (D) Based on the clusters identified in (C), median intensities for each marker were calculated and plotted as heat maps to identify the respective immune cell population. (E) Heat map of surface marker expression (median intensity) for IdU⁺ and IdU⁻ neutrophils, showing differentially expressed markers (black arrows). (C-E) Data are representative of one out of two independent experiments (n=3) (See also FIG. 12).

FIG. 6. Identification of a proliferative neutrophil precursor that is found in clusters in close proximity with CAR cells. (A) BM Gr1⁺CD11b⁺ neutrophils of Fucci-(S-G2-M) (#474) mice were gated accordingly and subjected to t-SNE dimensional reduction based on the expression of 11 markers. (B) Expression plot of Fucci-(S-G2-M) (#474) color mapped from blue (low expression) to red (high expression). (C) Differential expression of Fucci-(S-G2-M)⁺ (green) and Fucci-(S-G2-M)⁻ (grey) clusters (left) represented by overlaid histograms of indicated markers (middle) and plots (right). (D) Snapshot of cleared distal epiphysis of BM femur (250 μm thick vibratome section) showing Fucci-(S-G2-M)⁺ cells (green), neutrophils (S100A9, red), collagen (second harmonic generation, grey) and blood vessels (laminin, blue) (scale bar=300 μm). A zoomed-in view of a cluster is shown (right) (scale bar=20 μm). (E) Representative images, from 3 independent experiments, of Fucci-(S-G2-M)⁺S100A9⁺ cells in close proximity to CXCL12⁺ stromal cells (top, arrowheads) compared to Fucci-(S-G2-M)⁻S100A9⁺ cells (bottom, asterisk) (scale bar=10 μm). (F) The distance and mean distance to the nearest CAR cell (CXCL12⁺) or vessel (laminin⁺) (n=1893 preNeu and n=1509 Neu from the distal epiphyses of 3 BM femurs). Data reflect mean±SEM from three independent experiments. **, p<0.01; ***, p<0.001; ****, p<0.0001 (one-way ANOVA). (G) Comparison of BM preNeu in wildtype mice, S100a8^{cre}Cxcr4^{fl} mice and Cxcr4^{WHM/+} mice. Data are representative of at least two experiments. Results are expressed as a fold change in cell numbers±SD (n=10 mice per group). **, p<0.01; ****, p<0.0001 (See also FIG. 13).

FIG. 7. Transcriptomic analysis reveals distinct expression signatures during neutrophil development. (A-G) BM GMP, preNeu, immature Neu, mature Neu and blood Neu were sorted from three individual mice according to the gating strategy (A), and RNA was extracted for RNA-seq analysis (see gating strategy in FIG. 14A). (B) Wright-Giemsa staining of sorted populations (scale bar=10 μm). Data representative of 3 independent experiments. (C) PCA of gene expression. (D) Correlation matrix generated using Pearson's correlation coefficients that represents similarities of gene expression between subsets (low similarity=red, high similarity=yellow). (E) Heat-map of differentially-expressed genes between subsets among the 20% most variable genes (4820 genes out of 24098 detected transcripts). Genes clusters (1 to 7) were defined following hierarchical clustering and exported for gene ontology (G0) biological process analysis. (F) G0 biological process terms enriched in various indicated clusters. (G) Median gene expression (log₂ CPM) of indicated cluster-associated genes across sorted populations. (H) Gating strategy for identifying cell cycle stage using Fucci-(G0-G1) (#639)/Fucci-(S-G2-M) (#474) BM cells (left) and the representative proportions of each stage in indicated subsets (See also FIG. 14H). (I) Analysis of the in vitro proliferation assay of neutrophil subsets. Data are expressed as fold-change in numbers±SD (n=3) and is representative of four independent experiments. ****, p<0.0001 (one-way ANOVA) (See also FIG. 14).

FIG. 8. preNeu are committed towards the neutrophil lineage. (A) PCA of gene expression data from GMP and neutrophil and monocyte subsets. (B) Relative expression of S100a8 (log₂CPM) among GMPs and BM neutrophil subsets (n=3). (C) Strategy for genetic cell fate-mapping (left) and recombination frequency in the indicated populations (right). Results are expressed as mean±SD (n=3) and are representative of two independent experiments. (D) Intra-BM transfer of sorted Lyz2-GFP⁺ preNeu into wild type recipients. Top-row: identification strategy of the different cell populations. Medium and bottom row: black dots represent transferred cells at day 1 (middle row) and day 2 (bottom row) after transfer. Data are representative of one out of five independent mice. (E) Kinetics of BrdU incorporation among neutrophil subsets after a single pulse of BrdU. Data are expressed as mean±SD (n≥3 per timepoint) and are representative of two experiments. (F) Myelodepletion of BM cell populations using 5-FU. Data are expressed as mean±SD (n≥3 per timepoint) and are representative of two experiments (See also Fig. S15).

FIG. 9. Functional maturation along neutrophil development. (A-B) Expression of genes (z-score normalized) encoding (A) myeloid development-related TFs and (B) granule production, assessed in GMPs and neutrophil subsets. (C) ROS biosynthetic process-related genes in GMPs and neutrophil subsets. (D) ROS production by neutrophils subsets assessed by flow cytometry using dihydrorhodamine 123 (DHR). Data are shown as a geometric mean±SD (n=3) and are representative of two independent experiments. **, p<0.01 (one-way ANOVA). (E) Phagocytosis of GFP⁺ E. coli, expressed as a percentage of GFP⁺cells±SD (n=3) and are representative of two independent experiments. *, p<0.05; ***, p<0.001 (one-way ANOVA). (F) Phagocytosis-related genes expression in GMP and neutrophil subsets. (G) Chemotaxis-related genes and their corresponding expression in GMP and neutrophil subsets. (H) (top) Experimental set-up of the laser-induced sterile injury model. (bottom) Maximum z-projected snapshots from time-lapse showing neutrophil subsets migration towards the laser burn (grey square) (bottom left) with corresponding cell tracks (bottom right). Scale bar=100 μm. Time, h:min. Data are representative of three independent experiments.

FIG. 10. C/EBPE-deficiency impairs the development of preNeu and downstream neutrophil populations. (A-B) Absolute counts of BM myeloid cell subsets in WT and Cebpe^{-/-} mice expressed as mean±SD (n=5) and are representative of two independent experiments. (C) (top) Experimental set-up and (bottom) percentage contribution of various hematopoietic cells by WT CD45.1⁺ or Cebpe^{-/- CD}45.2⁺ cells expressed as mean±SD (n=5) and are representative of two independent experiments. (D) Absolute counts of infiltrated skin neutrophils. (E) (left) Representative (n=3) photographs showing RPA-induced leakage. Insets, pixel classification: leakage, white; no leakage, black. (right) Measurement of total Evans blue dye in mouse ears. Results are pooled from two experiments, expressed as mean±SEM (n=9-12 per group).**, p<0.001 (Student's t test). (F) Absolute counts of mature neutrophils in the blood and peritoneum of WT and Cebpe^{-/-} mice. (G) Bacteria CFU quantification of blood and peritoneal fluid 24 hours after mid-grade CLP. Results are expressed in mean±SD (n=4-10 per group) and are representative of two experiments. ***, p<0.001 (Student's t test).

FIG. 11. Immature Neutrophils can be distinguished from mature Neutrophils through CD101 expression and are associated with tumor progression. (A-B) Absolute counts of the expansion of preNeu in the BM (A) and spleen (B) under cecal ligation and puncture (CLP) mid-grade sepsis. Results are expressed as mean±SEM (n=3-5 per condition). ***, p<0.001 (Student's t test) and are representative of two experiments. (C-D) Absolute counts of the expansion of preNeu in the BM (C) and spleen (D) in tumor-bearing mice. Results are pooled from three experiments and are expressed as mean±SEM (n=15-16 per condition). ****, p<0.0001 (Student's t test) and are representative of three experiments. (E) CXCR2 expression among total neutrophils (Lin⁻CD115⁻SiglecF⁻Gr1⁺CD11b⁺) in BM, blood and pancreas orthotopic tumors. Data are representative of three independent experiments. (F) Gene expression of Cd101 (log₂CPM) in BM neutrophil subsets. Results are expressed as mean±SD (n=3). ****, p<0.0001 (one-way ANOVA). (G) Representative FACS plots of immature (red) and mature Neu (orange) in BM, spleen and blood. Histograms represent corresponding CXCR2 expression. Lineage markers include: B220, NK1.1, CD90.2, CD115, Siglec-F and MHCII. (H-I) Absolute number of immature and mature Neu present in blood (H) and pancreas (I) of naïve and tumor-bearing mice. Data are expressed as mean (n=15-16 per group). ***, p<0.001, ****, p<0.0001 (one-way ANOVA) (See also FIG. 16). (J) Graph showing the correlation between blood and pancreas immature neutrophils. Data are pooled from three independent experiments. Significance was determined by a Pearson correlation test. (K-M) Tumor-bearing mice were split into two groups based on the median tumor weight. (K-L) Representative FACS plots of blood and pancreas immature and mature Neu in naïve mice, and in mice carrying a low or high tumor burden. (M) Pancreas mass from mice carrying orthotopic tumors are separated into two groups: top 50% pancreas mass are considered as high tumor burden, while bottom 50% pancreas mass are considered as low tumor burden. Results are pooled from three independent experiments. (N) Absolute number of blood immature and mature Neu between mice carrying a low or high tumor burden. (O) Graph showing the correlation between blood immature Neu and pancreas weight of tumor bearing mice. Data are pooled from three independent experiments. Significance was determined by a Pearson correlation test.

FIG. 12 (related to FIG. 5): Mass cytometry reveals proliferative myeloid cells with distinct phenotypic signatures. (A) Surface marker expression levels of IdU⁺ and IdU⁻ basophils, eosinophils and Ly6C^{hi} monocytes. Arrows indicate differentially expressed surface markers.

FIG. 13 (related to FIG. 6): Identification of transitional pre-monocytes (tpMo) through their proliferation activity. (A) BM Ly6C^{hi} monocytes of Fucci-(S-G2-M) (#474) mice were gated and subjected to t-SNE dimensional reduction based on the expression of seven markers. (B) Expression level plot of Fucci-(S-G2-M) (#474) color mapped from blue (low expression) to red (high expression). (C) Differential expression levels of Fucci-(S-G2-M)⁺ (green) Fucci-(S-G2-M⁾⁻ (grey) clusters (left) represented by plotting CXCR4 against CD11b (middle) and overlaid histograms of indicated markers (right).

FIG. 14 (related to FIG. 7): Transcriptomic analysis reveals distinct expression signatures during neutrophil development. (A) Gating strategy of BM GMP and neutrophil subsets (preNeu, immature and mature Neu). (B) Gating strategy of spleen neutrophil subsets (preNeu, immature and mature Neu). (C-D) Absolute counts of (C) BM or (D) spleen neutrophil subsets. (E) Heat map of relative surface marker expression levels between BM and splenic neutrophil subsets. (F) Volcano plots depicting the number of differentially expressed genes together with log2 fold change between GMP and preNeu, preNeu and Immature Neu, immature and mature Neu and mature and blood Neu versus the −log10 FDR. (G) Cell cycle related gene expression in GMPs and neutrophil subsets. (H) Gating strategy for identifying cell cycle stage using Fucci-(G0-G1) (#639)/Fucci-(S-G2-M) (#474) BM cells (left) and (I) the representative proportions of each stage in the indicated subsets (right). (J) Colony forming assay of the sorted BM GMP and neutrophil subsets supplemented with the indicated cytokines. Results are representative of three independent experiments. Scale bar=50 μm.

FIG. 15 (related to FIG. 8): preNeu are committed towards the neutrophil lineage. (A) Computationally determined developmental path using the optimal leaf ordering (OLO) algorithm, that starts with GMP and ends with blood Neu as the most mature population. (B) Gene expression levels of S100a8 (log₂CPM) in indicated subsets. (C) Gene expression levels of Lyz2 (log₂CPM) in indicated subsets. (D) Fate mapping recombination frequency in the indicated subsets. Results are expressed as mean±SD (n=3) and are representative of two independent experiments. (E-K) Unsupervised analysis of healthy human bone marrow. (E) t-SNE visualization of human BM showing the various identified immune subsets in the sample. (F) Representative plot of the IdU incorporation in total neutrophils and (G) the differentially expressed markers (indicated by black arrows) between IdU+ and IdU− neutrophils. (H) Gating Strategy of human BM neutrophil subsets. (I) Wright-Giemsa staining of the neutrophil subsets (scale bar=10 μm). (J) Representative bi-axial plot of the neutrophil subsets in healthy human whole blood. (K) Surface marker expression levels of human BM neutrophil subsets. Data are represented as median intensity.

FIG. 16 (related to FIG. 11): Immature neutrophils are mobilizable and motile during inflammation. (A-B) Representative FACS plots of BM and spleen preNeu expansion in 2 weeks after CLP-induced sepsis (A) and 3 weeks after orthotopic tumor transplant (B) models. (C) Representative FACS plots of blood immature and mature Neu 24 h after G-CSFcx stimulation. (D) Mobilization kinetics of immature and mature Neu after G-CSFcx administration. Results are expressed as mean±SD (n=4 per time point). *, p<0.05; ****, p<0.0001 (one-way ANOVA), and are representative of two independent experiments. (E) (top) Experimental set-up of the laser-induced sterile injury model. (bottom) Maximum z-projected snapshots from time-lapse showing neutrophil subsets migration towards the laser burn (grey square) (bottom left) with corresponding cell tracks (bottom right). Scale bar=100 μm. Time, h:min. Data are representative of three independent experiments. (F-G) Graph showing the correlation between blood (F) mature Neu or (G) Ly6Chi monocytes and pancreas weight of tumor bearing mice. Data are pooled from three independent experiments. Significance was determined by a Pearson correlation test.

FIG. 17. Proliferative potential of Mouse Neutrophil Precursors. (A) Representative gating Strategy of neutrophil precursors and subsets using flow cytometric analysis of murine mouse bone marrow. (B) Colony forming unit (CFU) assay of indicated neutrophil precursors over 6 days. Black scale bars=20 μM. White scale bars=100 μM. Data is representative of three independent experiments. (C) Proliferation assay of indicated neutrophil precursors over 4 days. Data is expressed as mean (n=3) and is representative of three independent experiments. **=p<0.01, (Mann-Whitney test). (D) In vivo transfer of sorted GFP+ proNeu #2. Cells were sorted according to the gating strategy shown in (A). Sorted cells were then transferred intra-femorally and tracked across time as indicated. Data is representative of at least three independent experiments.

FIG. 18. Identification of Corresponding Neutrophil Precursors in Humans. Representative gating Strategies of neutrophil precursors and subsets using flow cytometric analysis of human (A) Cord blood, (B) Fetal bone marrow and (C) Adult bone marrow. All samples were processed and stained in the same way. Samples were lysed in 1× RBC lysis buffer (eBioscience) for 5 min and preincubated with human Fc blocker for 20 min before staining with fluorophore-conjugated antibodies. Data is representative of (A)>10 donors, (B) 1 donor, (C) 3 donors.

FIG. 19. In vivo proliferative and differentiation potential of preNeus. (A) In vivo transfer of sorted GFP+ preNeus into wild-type mice over 3 days. Cells were sorted according to the gating strategy shown in FIG. 17. Sorted cells were then transferred intra-femorally and tracked across time as indicated. Data is representative of at least three independent experiments.

FIG. 20. Transcriptional Regulation of Neutrophil Precursors. (A) Top 10 variable genes expressed by the indicated subsets. Data is obtained from 281 single-cell RNA-seq (Smart-seq2) and analysed using Seraut. (B) Violin plot of known transcription factors critical for neutrophil/monocyte fate decision. Values are expressed as raw UMI counts. (C) Heatmap of known neutrophil-related genes and their scaled expression values. Genes highlighted in light font (i.e. Gfi1, Far2, Per3, Camp, S100a8, S100a9, Ngp, Ltf, and Wfdc21) indicate exclusive genes and transcription factors to their respective neutrophil precursor population.

### DETAILED DESCRIPTION

Examples of the present disclosure will now be described with reference to the accompanying drawings. The terminology used herein is for the purpose of describing examples only and is not intended to limit the scope of the present disclosure. Additionally, unless defined otherwise, all technical and scientific terms used herein have the same meanings as commonly understood by one or ordinary skill in the art to which the present disclosure belongs.

Neutrophils are the most abundant immune cell type in human peripheral blood, and they act as the first responders during sterile and microbial insults. They elicit powerful effector functions to eliminate foreign threats and play crucial roles in tissue remodeling. Neutrophils are short-lived with an estimated half-life of 19 h in humans. Therefore, neutrophils must be constantly replenished as an impairment in their production and migration leads to neutropenia and life-threatening conditions.

Historically, neutrophil development has been defined using histological staining and electron microscopy into stages based on size, nucleus morphology and cytosol coloration. After maturation, neutrophils are retained in the bone marrow through CXCR4 chemokine receptor signaling while CXCR2 signaling drives their release into the circulation. During inflammation, increased amounts of granulocyte-colony stimulating factor (G-CSF) can potentiate neutrophil mobilization from the bone marrow by lowering the threshold of its release and increasing the amounts of mobilizing signals (i.e. CXCL1).

It is believed that neutrophils consist of a homogenous population. However, this view is rapidly evolving due to increasing reports of neutrophil heterogeneity. Notably, studies in the art focused primarily on the phenotype of circulating neutrophils but not their ontogeny. Therefore, the functional heterogeneous populations at the early maturation stages remains undefined. Myeloid cell development begins with the common myeloid progenitor (CMP), which gives rise to the granulocyte-monocyte progenitor (GMP). GMPs have also been shown to give rise to the common DC progenitor (CDP) and common monocyte progenitor (cMoP) that only form DCs or monocytes respectively. However, the developmental trajectory from GMP to functionally mature neutrophils remain poorly defined. To address this, multiparameter analytical techniques were utilized in the present disclosure to investigate the differentiation pathways and functional properties of neutrophil subsets in steady and inflammatory states.

### Definitions

As used herein, the term “about” may refer to +/−5% of the stated value, or +/−4% of the stated value, or +/−3% of the stated value, or +/−2% of the stated value, or +/−1% of the stated value, or +/−0.5% of the stated value.

Throughout the specification, unless the context requires otherwise, the word “comprise” or variations such as “comprises” or “comprising”, will be understood to imply the inclusion of a stated integer or group of integers but not the exclusion of any other integer or group of integers. Throughout the specification, unless the context requires otherwise, the word “include” or variations such as “includes” or “including”, will be understood to imply the inclusion of a stated integer or group of integers but not the exclusion of any other integer or group of integers.

Biomarkers or a component thereof includes but are not limited to polypeptides (e.g. cell surface proteins) and polynucleotides (e.g. DNA and RNA).

As used herein, the term “treatment”, “treat” and “therapy”, and synonyms thereof refer to both therapeutic treatment and prophylactic or preventative measures, wherein the object is to prevent, slow down (lessen), or cure a medical condition, which includes but is not limited to diseases (such as autoimmune diseases or cancer), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

As used herein, the term “therapeutically effective amount” of a compound will be an amount of an active agent that is capable of preventing or at least slowing down (lessening) a medical condition, such as autoimmune diseases, inflammation and cancer. Dosages and administration of compounds, compositions and formulations of the present disclosure may be determined by one of ordinary skill in the art of clinical pharmacology or pharmacokinetics. See, for example, Mordenti and Rescigno, (1992) Pharmaceutical Research. 9:17-25; Morenti et al., (1991) Pharmaceutical Research. 8:1351-1359; and Mordenti and Chappell, “The use of interspecies scaling in toxicokinetics” in Toxicokinetics and New Drug Development, Yacobi et al. (eds) (Pergamon Press: NY, 1989), pp. 42-96. An effective amount of the active agent of the present disclosure to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

As used in the specification herein, the term “subject” includes patients and non-patients. The term “patient” refers to individuals suffering or are likely to suffer from a medical condition, while “non-patients” refer to individuals not suffering and are likely to not suffer from a medical condition. “Non-patients” include healthy individuals. The term “subject” includes humans and animals. Animals include murine and the like. “Murine” refers to any mammal from the family Muridae, such as mouse, rat, and the like.

As used in the specification herein, agents for detecting biomarkers in the present disclosure refer to any compound, molecule and/or system that functions to detect the presence/absence and/or expression or level thereof of biomarkers in the present disclosure. Such agents are capable of detecting and/or binding directly or indirectly to a biomarker. In the present disclosure, additional moieties may be required to enhance the detection of the biomarkers, for example, by/through amplifying optical diffraction. Examples of agents and the additional moieties include but are not limited to proteins (for example antigen binding proteins such as antibodies or fragments thereof, enzymes such as horseradish peroxides and alkaline phosphatase, and the like), polynucleotides (for example aptamers), and small molecules (for example metallic nanoparticles).

As used herein, an “expression” refers to both genotypic as well as phenotypic expression of biomarkers in the present disclosure.

A “biomarker” refers to a molecule, for example a protein, carbohydrate structure, glycolipid, glycoprotein (including cell surface glycoprotein), or gene (or nucleic acid encoding the gene), the expression of which in or on a cell (or sample) derived from a subject (such as a mammalian tissue) can be detected by standard methods in the art (as well as those disclosed herein). In some examples, a biomarker may be any molecule that may serve as an identifier (i.e. marker) of a target of interest. Thus, in some examples, a biomarker may be a cell surface glycoprotein, transcription factors, and the like. In some examples, the biomarker may be a cell surface glycoprotein such as but is not limited to CD marker. “CD marker” as used herein refers to biomarkers associated with a cell, as recognised by sets of antibodies (as exemplified in Tables 1 and 2), which may be used to identify, detect, select, sort, and/or isolate the cell type, stage of differentiation, and activity state of a cell.

In some examples, when the biomarker is a cell surface marker or glycoprotein, the expression of the marker may be denoted in accordance to the acceptable denotation known in common general knowledge. For example, for a cell surface glycoprotein CD10, a CD10⁺ refers to the cell positively expresses CD10, a CD10⁻ refers to the cell not expressing detectable CD10, CD10^{lo} refers to the cell expressing low CD10, CD10^{int} refers to the cell expressing intermediate CD10, and CD10^{hi} refers to the cell expressing high CD10.

The present disclosure provides for antigen binding proteins including but not limited to polyclonal and/or monoclonal antibodies and fragments thereof, and immunologic binding equivalents thereof, which are capable of specifically binding to a target (such as a polypeptide target) and fragments thereof. Such antigen binding proteins thus include for example, but are not limited to polyclonal, monoclonal, chimeric, single chain, Fab fragments, and a Fab expression library. As used herein, “antibody” refers to a protein comprising one or more polypeptides substantially encoded by immunoglobulin genes or fragments of immunoglobulin genes. The recognised immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. An antibody may be specific for a particular antigen.

A “monoclonal antibody” refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bi-specific (chimeric) monoclonal antibody.

As used in the specification herein, the term “immobilized” refers to being bound directly or indirectly to a surface of, e.g., a device, including attachment by covalent binding or noncovalent binding (e.g., hydrogen bonding, ionic interactions, van der Waals forces, or hydrophobic interactions).

As used in the specification herein, neutrophils include pro-neutrophils (or also referred to as “proNeu”), pre-neutrophils (or also referred to as “preNeu”), immature neutrophils, and mature neutrophils.

Methods of the present disclosure include but are not limited to in vivo, in vitro and ex vivo methods.

Throughout this disclosure, certain examples may be disclosed in a range format. It should be understood that the description in range format is merely for convenience and brevity and should not be construed as a limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. Ranges are not limited to integers, and can include decimal measurements. This applies regardless of the breadth of the range.

### EXAMPLES OF THE PRESENT DISCLOSURE

The present disclosure seeks to provide a method of categorizing/characterising neutrophils into neutrophil subtypes and separating and/or isolating/enriching the same. The present disclosure also seeks to provide kits, and therapeutic, diagnostic and prognostic methods related to neutrophil subtypes.

According to an aspect of the present invention, there is provided a method of characterising and/or separating neutrophils, the method comprising characterising and/or separating the neutrophils into a first neutrophils population comprising proliferative neutrophils and a second neutrophils population comprising mature neutrophils, according to the expression of CD101 on the neutrophils.

In some examples, the proliferative neutrophils may be pro-neutrophils and pre-neutrophils. As used herein, the term “proliferative” refers to the ability of a cell to divide and therefore produce more cells of the same or more differentiated type.

Thus, proliferative neutrophils refer to hematopoietic cells that have committed to the neutrophil lineage, but still retain their ability to divide and produce more of the same cells (i.e. more pro-neutrophils and/or pre-neutrophils) or more differentiated types (i.e. immature neutrophils and/or mature neutrophils).

In some examples, the first population expresses CD101⁻ and the second population expresses CD101⁺.

As shown in the experimental section, when the neutrophils are human neutrophils (such as neutrophils that are from a population of cells obtained from a human subject), the method may further comprise characterising or separating the neutrophils into neutrophil subtypes according to the expression of CD10 on the neutrophils, wherein the first population comprising proliferative neutrophils are CD10⁻ CD101⁻, and the second population comprising mature neutrophils are CD10⁺CD101⁺. At the same time, in human, the second neutrophils population further comprises immature neutrophils. In human, the immature neutrophils are CD10⁻CD101⁺.

The inventors of the present disclosure surprisingly found two subtypes of neutrophils that are capable of proliferating under suitable conditions. These proliferative neutrophils are referred to in the present disclosure as pro-neutrophils (also referred to as “proNeu”) and pre-neutrophils (also referred to as “preNeu”). Thus, in some examples, the proliferative neutrophils include pro-neutrophils and pre-neutrophils.

In some examples, the (human) pro-neutrophils and/or pre-neutrophils are CD10⁻CD101⁻. Therefore, the method as described herein may further comprise characterising the proliferative neutrophils (or pro-neutrophils and/or pre-neutrophils) to be CD10⁻CD101⁻.

As exemplified in the Experimental Section, pro-neutrophils may be characterised by their ability to proliferate as well as their expression of biomarkers such as, but is not limited to, CD101, CD10, CD16, CD34, CD66b, CD15, CD71, CD49d, CD11b, CXCR2, and the like. In some examples, the pro-neutrophils may express one or more biomarkers such as but is not limited to CD101⁻, CD10⁻, CD16⁻, CD34⁻, CD66b⁺, CD15⁺, CD71⁺, CD49d⁺, CD11b⁻, CXCR2⁻, and the like. In some examples, the pro-neutrophils may express or be characterised by CD34⁻ CD66b⁺CD15⁺CD71⁺CD4d⁺CD101⁻CD11b⁻. Other biomarkers that may characterise pro-neutrophils include any other biomarkers discussed in the experimental section of the present disclosure.

Accordingly, in some examples, the method may further comprise characterising and/or separating the pro-neutrophils (i.e. proNeu) according to the expression of one or more biomarkers such as, but is not limited to, CD101, CD10, CD16, CD34, CD66b, CD15, CD71, CD49d, CD11b, CXCR2, and the like. In some examples, the method may further comprise characterising and/or separating the pro-neutrophils based on their expression of one or more of CD101⁻, CD10⁻, CD16⁻, CD34⁻, CD66b⁺, CD15⁺, CD71⁺, CD49d⁺, CD11b⁻, CXCR2⁻, and the like. In some examples, other biomarkers used in the experimental section of the present disclosure may be included in the method as described herein.

Pre-neutrophils may also be characterised by their ability to proliferate as well as their expression of biomarkers such as but is not limited to, CD101, CD10, CD16, CD34, CD66b, CD15, CD71, CD49d, CD11b, CXCR2, and the like. In some examples, the pre-neutrophils may express one or more biomarkers such as, but is not limited to, CD101⁻, CD10⁻, CD16⁻, CD34⁻, CD66b⁺, CD15⁺, CD71⁺, CD49d⁺, CD11b⁺, CXCR2⁻, and the like. In some examples, the pre-neutrophils may express or be characterised by CD66b⁺CD15⁺CD71⁺CD4d⁺CD101⁻CD11b⁺. Other biomarkers that may characterise pre-neutrophils include any other biomarkers discussed in the experimental section of the present disclosure.

Accordingly, in some examples, the method may further comprise characterising and/or separating the pre-neutrophils according to the expression of one or more biomarkers such as, but is not limited to, CD101, CD10, CD16, CD34, CD66b, CD15, CD71, CD49d, CD11b, CXCR2, and the like. In some examples, the method may further comprise characterising and/or separating pre-neutrophils based on their expression of one or more biomarkers such as, but is not limited to, CD101⁻, CD10⁻, CD16⁻, CD34⁻, CD66b⁺, CD15⁺, CD71⁺, CD49d⁺, CD11b⁺, CXCR2⁻, and the like. In some examples, other biomarkers used in the experimental section of the present disclosure may be included in the method as described herein.

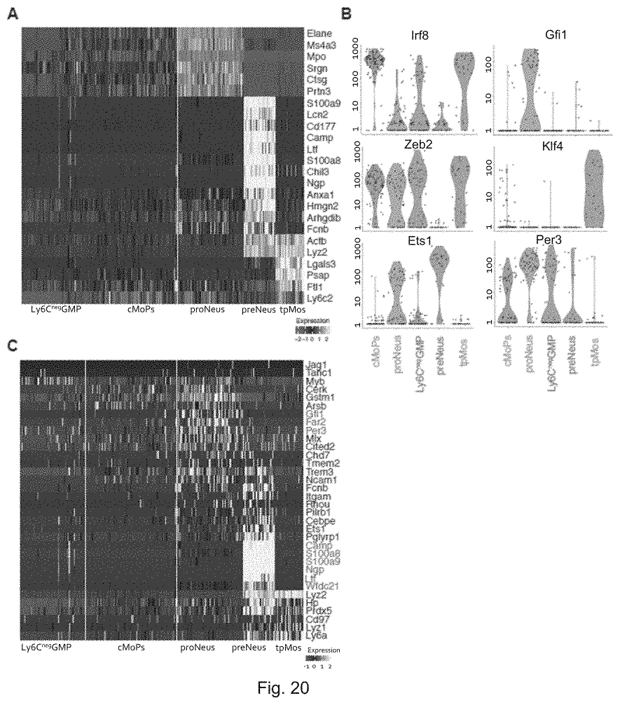

In some examples, proliferative neutrophils (such as pro-neutrophils and pre-neutrophils) may be characterised and/or separated based on the expression of transcription factors (or genes) such as but not limited to Gfi1, far2, Per3, Camp, S100a8, S100a9, Ngp, Ltf, Wfdc21, and the like. In some examples, transcription factors (or genes) that may be used to distinguish pro-neutrophils and/or pre-neutrophils from immature neutrophils and/or mature neutrophils includes but is not limited to transcription factors (or genes) related to cell cycle and/or granule (such as primary granules). Examples of transcription factors (or genes) related to cell cycle and/or granule (such as primary granules) include but is not limited to transcription factors and/or genes as disclosed herein in FIG. 20A. In some examples, the transcription factors (or genes) may include but is not limited to Elane, Ms4a3, Mpo, Srgn, Ctsg, Prtn3, S100a9, Lcn2, Cd177, Camp, Ltf, S100a8, Chil3, Ngp, Anxa1, Hmgn2, Arhgdib, Fcnb, Actb, Lyz2, Lgals3, Psap, Ftl1, Ly6c2, and the like. In some examples, the transcription factors (or genes) may include but is not limited to Elane, Mpo, Srgn, Ctsg, Prtn3, and the like.

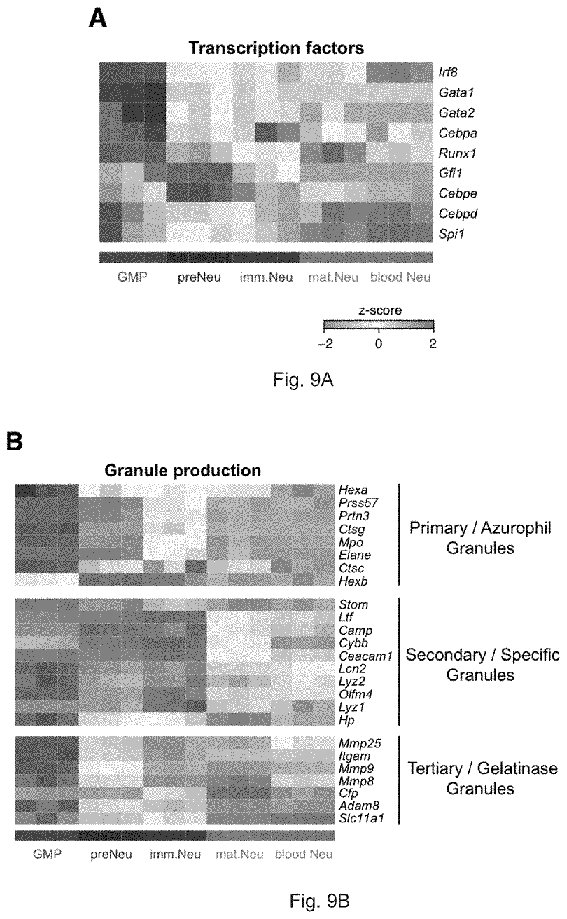

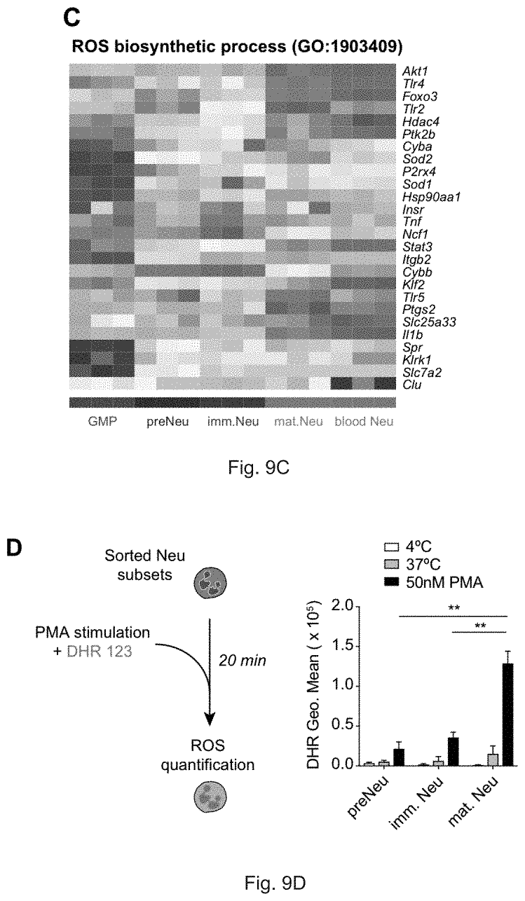

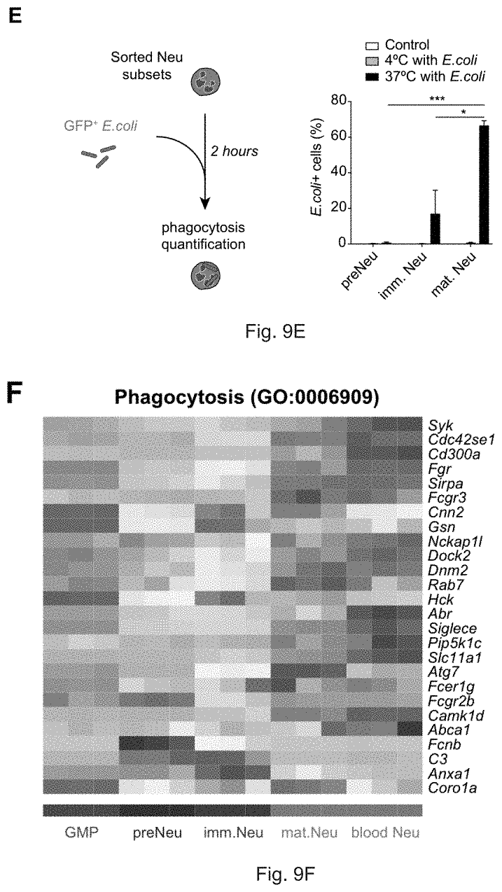

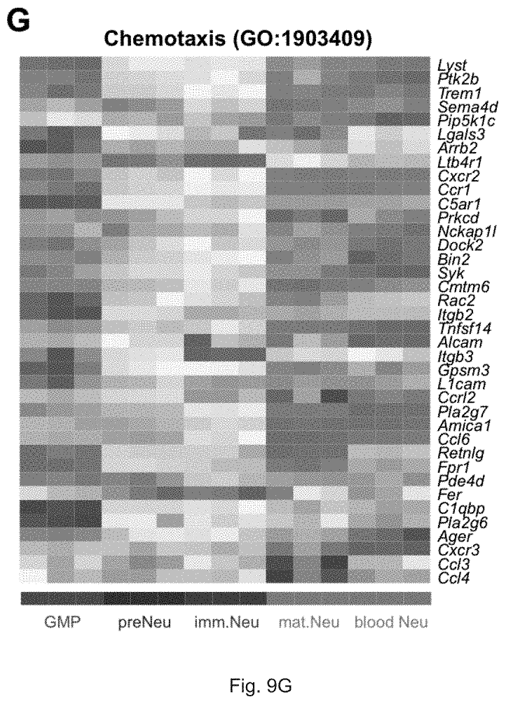

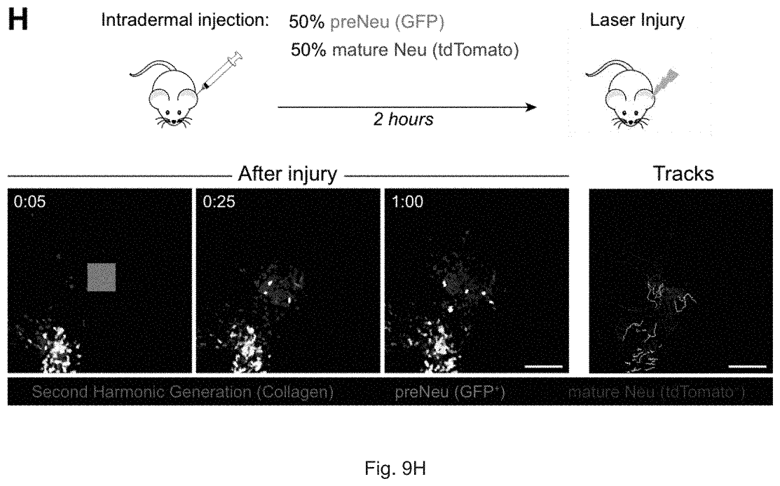

In some examples, immature and/or mature neutrophils may be characterised and/or separated based on their expression of transcription factors related to terminal granulopoiesis, neutrophil effector functions, such as but is not limited to production of reactive oxygen species (ROS), production of neutrophilic granules, phagocytosis, chemotaxis, and the like. For example, mature neutrophils may express transcription factors such as but is not limited to Cd101, Cebpd, Spi1 (PU.1), transcription factors recited in FIG. 9, and the like. In some examples, mature neutrophils may be characterised and/or separated based on their expression of transcription factor (or gene) such as but is not limited to Cd101. In some examples, mature neutrophils may be characterised and/or separated based on their expression of transcription factors related to ROS biosynthetic process such as but is not limited to Akt1, Tlr4, Foxo3, Tlr2, Hdac4, Ptk2b, Stat3, Itgb2, Cybb, Klf2, Tlr5, Ptgs2, Slc25a33, Il1b, Clu, and the like. In some examples, immature and/or mature neutrophils may be characterised and/or separated based on their expression of transcription factors related to tertiary. Gelatinase granules such as but is not limited to Mmp25, Itgam, Mmp9, Mmp8, Cfp, Adam8, Slc11a1, and the like. In some examples, mature neutrophils may be characterised and/or separated based on their expression of transcription factors related to phagocytosis such as but is not limited to Syk, Cdc42se1, Cd300a, Fgr, Sirpa, Fcgr3, Gsn, Nckap1I, Dock2, Dnm2, Rab7, Hck, Abr, Siglece, Pip5k1c, Slc11a1, Atg7, Fcer1g, camk1d, Abca1, Coro1a, and the like. In some examples, mature neutrophils may be characterised and/or separated based on their expression of transcription factors related to chemotaxis such as but is not limited to Lyst, Ptk2b, Trem1, Sema4d, Pip5k1c, Lgals3, Arrb2, Cxcr2, Ccr1, C5ar1, Prkcd, Nckap1l, Dock2, Bin2, Syk, Cmtm6, Rac2, Itgb2, Tnfsf14, Alcam, Itgb3, Gpsm3, L1cam, Ccrl2, Pla2g7, Amica1, Ccl6, Retnlg, Fpr1, Ager, Cxcr3, Ccl3, Ccl4, and the like.

In humans, the immature neutrophils may be characterised by the expression of one or more biomarkers such as, but is not limited to CD101, CD10, CD16, CD34, CD66b, CD15, CD71, CD49d, CD11b, CXCR2, and the like. In some examples, the immature neutrophils may express one or more biomarkers such as, but is not limited to, CD101⁺, CD10⁻, CD16⁻, CD34⁻, CD66b⁺, CD15⁺, CD71⁻, CD49d^{lo}, CD11b⁺, CXCR2⁻, and the like. Other biomarkers that may characterise immature neutrophils include any other biomarkers discussed in the experimental section of the present disclosure.

Accordingly, in some examples, the method may further comprise characterising and/or separating the immature neutrophils according to the expression of one or more biomarkers such as, but is not limited to, CD101, CD10, CD16, CD34, CD66b, CD15, CD71, CD49d, CD11b, CXCR2, and the like. In some examples, the method may further comprise characterising and/or separating immature neutrophils based on their expression of one or more biomarkers such as, but is not limited to, CD101⁺, CD10⁻, CD16⁻, CD34⁻, CD66b⁺, CD15⁺, CD71⁻, CD49d^{lo}, CD11b⁺, CXCR2⁻, and the like. In some examples, other biomarkers used in the experimental section of the present disclosure may be included in the method as described herein.

In humans, the mature neutrophils may be characterised by the expression of one or more biomarkers such as, but is not limited to CD101, CD10, CD16, CD34, CD66b, CD15, CD71, CD49d, CD11b, CXCR2, and the like. In some examples, the mature neutrophils may express one or more biomarkers such as, but is not limited to, CD101⁺, CD10⁺, CD16⁺, CD34⁻, CD66b⁺, CD15⁺, CD71⁻, CD49d^{lo}, CD11b⁺, CXCR2⁺, and the like. Other biomarkers that may characterise mature neutrophils include any other biomarkers discussed in the experimental section of the present disclosure.

Accordingly, in some examples, the method may further comprise characterising and/or separating the mature neutrophils according to the expression of one or more biomarkers such as, but is not limited to, CD101, CD10, CD16, CD34, CD66b, CD15, CD71, CD49d, CD11b, CXCR2, and the like. In some examples, the method may further comprise characterising and/or separating mature neutrophils based on their expression of one or more biomarkers such as, but is not limited to, CD101⁺, CD10⁺, CD16⁺, CD34⁻, CD66b⁺, CD15⁺, CD71⁻, CD49d^{lo}, CD11b⁺, CXCR2⁺, and the like. In some examples, other biomarkers used in the experimental section of the present disclosure may be included in the method as described herein.

As shown in the experimental section, when the neutrophils are murine neutrophils (such as neutrophils that are from a population of cells obtained from a murine subject), the method may further comprise detecting expression of cKit on the neutrophils and characterising the neutrophils into neutrophil subtypes according to the expression of cKit on the neutrophils, wherein the first population comprising proliferative neutrophils are cKit^{hi}CD101⁻ or cKit^{int}CD101⁻ or cKit^{lo}CD101⁻ and the second population comprising mature neutrophils are cKit⁻CD101⁺ (i.e. cKit^{(negative)}CD101⁺) At the same time, in murine, the first neutrophils population further comprises immature neutrophils. In rodents (such as murine or mouse), the immature neutrophils are cKit^{lo}CD101⁻.

In murine, the pro-neutrophils may be cKit^{hi}CD101⁻ and pre-neutrophils may be cKit^{lo}CD101⁻ or cKit^{int}CD101⁻. Therefore, the method as described herein may further comprise characterising and/or separating the proliferative neutrophils (or pro-neutrophils and/or pre-neutrophils) to be cKit^{hi}CD101⁻, cKit^{int}CD101⁻ or cKit^{lo}CD101⁻.

As exemplified in the Experimental Section, (murine) pro-neutrophils may be characterised by their ability to proliferate as well as their expression of biomarkers such as, but is not limited to, CD101, cKit, Ly6C, CD106, SiglecF, CD115, CD205, CD11b, Gr1, CXCR4, and the like. In some examples, the pro-neutrophils may express one or more biomarkers such as, but is not limited to, CD101⁻, cKit^{Hi}, Ly6C⁺, CD106⁺, SiglecF⁻, CD115⁻, CD205⁻, CD11b^{lo}, Gr1^{Lo}, CXCR4^{Hi}, and the like. In some examples, the pro-neutrophils may be characterised by cKit^{hi}Ly6C⁺CD106⁺CD115⁻ CD205⁻CD11b^{lo}Gr1^{lo}. Other biomarkers that may characterise pro-neutrophils include any other biomarkers discussed in the experimental section of the present disclosure.

Accordingly, in some examples, the method may further comprise characterising and/or separating the pro-neutrophils (i.e. proNeu) according to the expression of one or more biomarkers such as, but is not limited to, CD101, cKit, Ly6C, CD106, SiglecF, CD115, CD205, CD11b, Gr1, CXCR4, and the like. In some examples, the method may further comprise characterising and/or separating the pro-neutrophils based on their expression of one or more of CD101⁻, cKit^{Hi}, Ly6C⁺, CD106⁺, SiglecF⁻, CD115⁻, CD205⁻, CD11b^{Lo}, Gr1^{Lo}, CXCR4^{Hi}, and the like. In some examples, other biomarkers used in the experimental section of the present disclosure may be included in the method as described herein.

In some examples, (murine) pre-neutrophils may also be characterised by their ability to proliferate as well as their expression of biomarkers such as but is not limited to, CD101, cKit, Ly6C, CD106, SiglecF, CD115, CD205, CD11b, Gr1, CXCR4, and the like. In some examples, the pre-neutrophils may express one or more biomarkers such as, but is not limited to, CD101⁻, cKit^{lo} or cKit^{int}, Ly6C⁺, CD106⁺⁺, SiglecF⁻, CD115⁻, CD205⁺, CD11b^{Hi}, Gr1^{Hi}, CXCR4^{Hi}, and the like. In some examples, the pre-neutrophils may be characterised by cKit^{lo}Ly6C⁺SiglecF⁻CD115⁻ CD205⁺CD11b^{hi}Gr1^{hi}CXCR4^{hi}. Other biomarkers that may characterise pre-neutrophils include any other biomarkers discussed in the experimental section of the present disclosure.

Accordingly, in some examples, the method may further comprise characterising and/or separating the pre-neutrophils according to the expression of one or more biomarkers such as, but is not limited to, CD101, cKit, Ly6C, CD106, SiglecF, CD115, CD205, CD11b, Gr1, CXCR4, and the like. In some examples, the method may further comprise characterising and/or separating pre-neutrophils based on their expression of one or more biomarkers such as, but is not limited to, CD101⁻, cKit^{lo} or cKit^{int}, Ly6C⁺, CD106⁺⁺, SiglecF⁻, CD115⁻, CD205⁺, CD11b^{Hi}, Gr1^{Hi}, CXCR4^{Hi}, and the like. In some examples, other biomarkers used in the experimental section of the present disclosure may be included in the method as described herein.

In some examples, the (murine) immature neutrophils may be characterised by the expression of one or more biomarkers such as, but is not limited to CD101, cKit, Ly6C, CD106, SiglecF, CD115, CD205, CD11b, Gr1, CXCR4, and the like. In some examples, the immature neutrophils may express one or more biomarkers such as, but is not limited to, CD101⁻, cKit^{lo} or cKit^{int}, Ly6C⁺, CD106⁺, SiglecF⁻, CD115⁻, CD205⁺, CD11b^{Hi}, Gr1^{Hi}, CXCR4^{lo}, and the like. Other biomarkers that may characterise immature neutrophils include any other biomarkers discussed in the experimental section of the present disclosure.

Accordingly, in some examples, the method may further comprise characterising and/or separating the immature neutrophils according to the expression of one or more biomarkers such as, but is not limited to, CD101, cKit, Ly6C, CD106, SiglecF, CD115, CD205, CD11b, Gr1, CXCR4, and the like. In some examples, the method may further comprise characterising and/or separating immature neutrophils based on their expression of one or more biomarkers such as, but is not limited to, CD101⁻, cKit^{lo}, Ly6C⁺, CD106⁺, SiglecF⁻, CD115⁻, CD205⁺, CD11b^{Hi}, Gr1^{Hi}, CXCR4^{lo}, and the like. In some examples, other biomarkers used in the experimental section of the present disclosure may be included in the method as described herein.

In some examples, the (murine) mature neutrophils may be characterised by the expression of one or more biomarkers such as, but is not limited to CD101, cKit, Ly6C, CD106, SiglecF, CD115, CD205, CD11b, Gr1, CXCR4, and the like. In some examples, the mature neutrophils may express one or more biomarkers such as, but is not limited to, CD101⁺, cKit⁻, Ly6C⁺, CD106^{lo}, SiglecF⁻, CD115⁻, CD205⁺, CD11b^{Hi}, Gr1^{Hi}, CXCR4^{lo} and the like. Other biomarkers that may characterise and/or separate mature neutrophils include any other biomarkers discussed in the experimental section of the present disclosure.

Accordingly, in some examples, the method may further comprise characterising and/or separating the mature neutrophils according to the expression of one or more biomarkers such as, but is not limited to, CD101, cKit, Ly6C, CD106, SiglecF, CD115, CD205, CD11b, Gr1, CXCR4, and the like. In some examples, the method may further comprise characterising and/or separating mature neutrophils based on their expression of one or more biomarkers such as, but is not limited to, CD101⁺, cKit⁻, Ly6C⁻, CD106^{lo}, SiglecF⁻, CD115⁻, CD205⁺, CD11b^{Hi}, Gr1^{Hi}, CXCR4^{lo} and the like. In some examples, other biomarkers used in the experimental section of the present disclosure may be included in the method as described herein.

According to another aspect of the present disclosure, there is provided a kit for separating neutrophils. In some examples, the kit may comprise an agent for detecting the expression of CD101 on the neutrophils. In some examples, the kit may further comprise a separator for separating a first population comprising proliferative neutrophils and a second population comprising mature neutrophils according to the expression of CD101 on the neutrophils.

In some examples, the first population may express CD101⁻ and the second population may express CD101⁺. In some examples, the kit may be for separating human neutrophils and the kit may further comprises an agent for detecting the expression of CD10 on the human neutrophils. In some examples, the separator may be adapted to separate the neutrophils according to the expression of CD10 on the neutrophils, wherein the first population may comprise proliferative neutrophils that may be CD10⁻CD101⁻, and the second population may comprise mature neutrophils that may be CD10⁺CD101⁺. In some example, the second population may further comprise immature neutrophils that may be CD10⁻CD101⁺.

In some examples, the agent for detecting the expression of CD10 may be an antibody adapted to target CD10. In some examples, the agent for detecting the expression of CD101 may be an antibody adapted to target CD101.

In some examples, the kit may further comprise an agent for detecting the expression on the neutrophils one or more biomarkers selected from a group consisting of CD49d, CD16, CXCR2, CD34, CD66, CD15, CD71, and CD11b. As such, in some examples, the separator may also be adapted to separate the neutrophils according to the expression of one or more of CD49d, CD16, CXCR2, CD34, CD66, CD15, CD71, and CD11b on the neutrophils. In such examples, the characteristics of the various neutrophils subtypes (i.e. proliferative neutrophils including pro-neutrophils and pre-neutrophils, immature neutrophils and mature neutrophils) may be as described herein above and in the experimental section.

In some examples, the kit may be for separating murine neutrophils. In such examples, the separator may be further adapted to separate the neutrophils according to the expression of CD101 and/or cKit. In some examples, the first population may comprise proliferative neutrophils that may be any one of cKit^{hi}CD101⁻, cKit^{int}CD101⁻, or cKit^{lo}CD101⁻ and the second population may comprise mature neutrophils that may be cKit⁻CD101⁺. In some examples, the first population may further comprise immature neutrophils, which may express cKit^{lo}CD101⁺.

In some examples, the agent for detecting the expression of CD101 and/or cKit may be an antibody adapted to target CD101 and/or cKit.

In some examples, the kit may further comprise an agent for detecting the expression on the neutrophils of one or more biomarkers selected from a group consisting of CD101, cKit, Ly6C, CD106, SiglecF, CD115, CD205, CD11b, Gr1, and CXCR4. In such examples, the separator may be adapted to separate the neutrophils according to the expression of CD101, cKit, Ly6C, CD106, SiglecF, CD115, CD205, CD11b, Gr1, and/or CXCR4 on the neutrophils. In such examples, the characteristics of the various neutrophils subtypes (i.e. proliferative neutrophils including pro-neutrophils and pre-neutrophils, immature neutrophils and mature neutrophils) may be as described herein above and in the experimental section.

According to another aspect of the present disclosure, there is provided a method of isolating and/or enriching a desired neutrophil. In some examples, the method may comprise: categorizing neutrophils in a sample into a first population comprising proliferative neutrophils and a second population comprising mature neutrophils according to the expression of CD101 on the neutrophils. In some examples, the method may also comprise isolating and/or enriching one or more neutrophil from the first population and/or the second population.

In some examples, the sample may be obtained from a human subject. In such examples, the method may further comprise categorizing the neutrophils according to the expression of CD10 on the neutrophils. In some examples, the first population may comprise proliferative neutrophils and may be CD10⁻CD101⁻ and the second population may comprise mature neutrophils and may be CD10⁺CD101⁺. In some examples, the second population may further comprise immature neutrophils and are CD10⁻CD101⁺.

In some examples, the method may comprise detecting expression of CD10 and/or CD101 with an agent adapted to target CD10 and/or CD101.

In some examples, the isolating of one or more neutrophil may comprise immobilizing the one or more neutrophil via an agent adapted to target CD10 and/or CD101.

In some examples, the method may further comprise the step of validating the neutrophil in the first and/or second population by detecting the expression of one or more biomarkers selected from a group consisting of CD49d, CD16, CXCR2, CD34, CD66, CD15, CD71, and CD11b. In such examples, the characteristics of the various neutrophils subtypes (i.e. proliferative neutrophils including pro-neutrophils and pre-neutrophils, immature neutrophils and mature neutrophils) may be as described herein above and in the experimental section.

In some examples, the sample may be obtained from a murine subject, and wherein the first population comprising proliferative neutrophils are CD101⁻, and the second population comprising mature neutrophils are CD101⁺. In some examples, the first population may further comprise immature neutrophils that are CD101⁻.

In some examples, the method may comprise detecting expression of CD101 with agents adapted to target CD101.

In some examples, the isolation of one or more desired neutrophil subtypes may be performed by methods known in the art. For example, the one or more desired neutrophils subtypes may be isolated through immobilizing the one or more desired neutrophil subtypes via agents adapted to target CD101.

In some examples, the method may further comprise the step of validating the desired neutrophil subtype by detecting the expression of one or more biomarkers selected from a group consisting of CD101, cKit, Ly6C, CD106, SiglecF, CD115, CD205, CD11b, Gr1, and CXCR4. In such examples, the characteristics of the various neutrophils subtypes (i.e. proliferative neutrophils including pro-neutrophils and pre-neutrophils, immature neutrophils and mature neutrophils) may be as described herein above and in the experimental section.

In some examples, the method may also comprise administering the subject with an agent capable of mobilising neutrophils, hematopoietic stem cells, and progenitor cells from bone marrow, stimulating neutrophils and/or inducing granulopoiesis. In some examples, the agent may include, but is not limited to, one or more of Plerixafor, granulocyte-colony stimulating factor (G-CSF) and/or interleukin 3 (IL-3) prior to obtaining the population of cells from the subject.

In some examples, the desired neutrophil subtype may be proliferative neutrophils, such as pro-neutrophils and/or pre-neutrophils.

In some examples, the method may further comprise the step of expanding the proliferative neutrophils (such as pro-neutrophils and/or pre-neutrophils) with one or more growth factors. As used herein, “growth factors” may include any biologically active molecule that is capable of facilitating or inducing a cell (such as neutrophil) to enter the cell division phase of a cell cycle (i.e. the S phase of a cell cycle). For example, the one or more growth factors may include, but is not limited to, interleukin 6 (IL-6), leukaemia inhibitory factor (LIF), stem cell factor (SCF), G-CSF, IL-3, and the like.

According to another aspect of the present disclosure, there is provided a composition comprising proliferative neutrophils. In some examples, the proliferative neutrophils may be CD10⁻CD101⁻.

According to another aspect of the present disclosure, there is provided a composition comprising a therapeutically effective amount of proliferative neutrophils for use in treatment. In some examples, the proliferative neutrophils may be CD10⁻ CD101⁻. In some examples, the composition may be for use in the treatment of immunodeficiency related diseases and/or disorders in a patient.

According to another aspect of the present disclosure, there is provided a composition comprising a therapeutically effective amount of proliferative neutrophils for enhancing the immune system of a subject and/or maintaining an immune response in the subject. In some examples, the proliferative neutrophils may be CD10⁻CD101⁻.

In some examples, the proliferative neutrophils may comprise pro-neutrophils and/or pre-neutrophils. As described herein, the pro-neutrophils may be CD101⁻CD10⁻CD16⁻CD34⁻CD66b⁺CD15⁺CD71⁺CD49d⁺CD11b⁻CXCR2⁻ and/or the pre-neutrophils are CD101⁻CD10⁻CD16⁻CD34⁻ CD66b⁺CD15⁺CD71⁺CD49d⁺CD11b⁺CXCR2⁻.

According to another aspect of the present disclosure, there is provided the use of proliferative neutrophils in the manufacture of a medicament for treating immunodeficiency related diseases and/or disorders in a patient. In some examples, the proliferative neutrophils may be CD10⁻CD101⁻.

According to another aspect of the present disclosure, there is provided a method of treating immunodeficiency related diseases and/or disorders in a patient, the method comprising administering a therapeutically effective amount of proliferative neutrophils to a patient. In some examples, the proliferative neutrophils may be CD10⁻ CD101⁻.

In some examples, the immunodeficiency related disease and/or disorders may be associated with cancer and/or infection. In some examples, the patient may be immunocompromised.

In some examples, the method may comprise administering a therapeutically effective amount of proliferative neutrophils to the patient as required. For example, the patient may require administration of the proliferative neutrophils every one (1) day to seven (7) days, once a week, once every two weeks, once every three weeks, once every four weeks (or a month), once a month, once every two months, and the like. In some examples, the patient may require administration of the proliferative neutrophils every two (2) to six (6) days, or every three (3) to five (5) days. In some examples, the method may comprise administering a therapeutically effective amount of proliferative neutrophils to the patient as required for a period of at least one week, at least two weeks, at least three weeks, at least four weeks, at least five weeks, at least one month, at least two months, at least three months, or at least for the duration of the patient being immunocompromised. In some examples, the patient may require administration of the proliferative neutrophils intermittently depending on the patient's immune state.

As used herein, “immunocompromised” refers to a state of being in a human patient where the immune system of the patient may not be considered optimal. For example, a human patient may be considered immunocompromised when the patient lacks certain component of the immune system. In some examples, the patient may be considered immunocompromised when the patient does not have the same amount of total neutrophil count or composition in a sample (such as bone marrow, spleen or blood sample) as a reference non-diseased (healthy or not immunocompromised) subject. For example, higher level of immature neutrophils in a patient as compared to a reference subject may indicate inflammation. “Reference subject” as used herein refers to a subject or individual of general population who is known to be non-diseased or at least do not have the same condition as the patient (i.e. subject suspected of or confirmed to be immunocompromised).

According to another aspect of the present disclosure, there is provided a method of enhancing the immune system of a patient, the method may comprise the step of: (a) obtaining a population of cells comprising neutrophils. In some examples, the method further comprises the step of (b) isolating proliferative neutrophils from the population of cells according to CD10 and/or CD101 expression on the neutrophils. In some examples, the method further comprises the step of (c) administering a therapeutically effective amount of the proliferative neutrophils to the patient. In some examples, the proliferative neutrophils may be CD10⁻CD101⁻.

In some examples, step (b) may further comprise detecting expression of CD10 and/or CD101 with agents adapted to target CD10 and/or CD101.

In some examples, the method may further comprise the step of expanding the pre-neutrophils prior to step (c).

In some examples, the proliferative neutrophils may be expanded with one or more growth factors. In some examples, the growth factors may be growth factors known in the art to encourage or facilitate or induce proliferation of neutrophils.

In some examples, the growth factors may include, but is not limited to, interleukin 6 (IL-6), leukaemia inhibitory factor (LIF), stem cell factor (SCF), G-CSF and IL-3.

In some examples, step (a) may comprise obtaining the population of cells comprising neutrophils from the patient. In some examples, the population of cells comprising neutrophils may be obtained from the bone marrow of the patient and/or from cord blood.

According to another aspect of the present disclosure, there is provided a method for diagnosing or prognosing a medical condition in a patient. In some examples, the method may comprise the step of (a) testing a sample comprising neutrophils obtained from a patient, to detect the expression of CD10 and/or CD101 on the neutrophils. In some examples, the method may comprise (b) measuring the levels of proliferative neutrophils, immature neutrophils and/or mature neutrophils in the sample, wherein proliferative neutrophils are CD10⁻CD101⁻, immature neutrophils are CD10⁻CD101⁺, and mature neutrophils are CD10⁺CD101⁺. In some examples, the method may further comprise the step of (c) comparing the levels of the proliferative neutrophils, immature neutrophils and/or mature neutrophils in the sample, to reference levels in a control to determine the absence or presence of the medical condition, or to predict the course of the medical condition.

In some examples, the sample may be a bone marrow sample and/or a spleen sample.

In some examples, where the sample is a bone marrow sample and/or a spleen sample, a level of proliferative neutrophils in the sample higher than the reference level in the control may indicate that the patient has an inflammatory medical condition.

In some examples, the inflammatory medical condition may be associated with an autoimmune disease, sepsis and/or cancer.

In some examples, a level of immature neutrophils in the sample higher than the reference level in the control may indicate that the patient has the medical condition. In some examples, the level of immature neutrophils may correlate with the progression of the medical condition.

In some examples, the sample may be a blood sample or a tumor sample.

In some examples, the medical condition may be cancer. For example, the cancer may include, but is not limited to, lung cancer, bladder cancer, head and/or neck cancer, breast cancer, esophageal cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, stomach cancer, prostate cancer, testicular cancer, ovarian cancer, cervical cancer, endometrial cancer, uterine cancer, pancreatic cancer, colon cancer, colorectal, gastric cancer, kidney cancer, bladder cancer, lymphoma cancer, spleen cancer, thymus cancer, thyroid cancer, brain cancer, neuronal cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, white blood cell cancer (e.g., lymphoma, leukaemia, etc.), hereditary non-polyposis cancer (HNPC), colitis-associated cancer, and the like. In some examples, the cancer may be pancreatic cancer.

According to another aspect of the present disclosure, there is provided a kit for detecting and/or predicting inflammation in a patient. In some examples, the kit may comprise an agent for detecting the expression of CD10 on neutrophils and/or an agent for detecting the expression of CD101 on neutrophils to measure the level of proliferative neutrophils in a sample taken from the patient. In some examples, the proliferative neutrophils may be CD10⁻CD101⁻. In some examples, the kit may further comprise a reference level for comparing the measured level of proliferative neutrophils. In some examples, a level of proliferative neutrophils in the sample higher than the reference level may indicate that the patient has an inflammatory medical condition.

According to another aspect of the present disclosure, there is provided a method of separating neutrophils, the method comprising the step of: separating the neutrophils into a first population comprising proliferative neutrophils and a second population comprising mature neutrophils, according to the expression of CD101 on the neutrophils.

In some examples, there is provided a method of separating neutrophils, the method comprising the steps of: (a) detecting expression of CD101 on neutrophils; and (b) separating the neutrophils into neutrophil subtypes comprising pre-neutrophils, immature neutrophils and mature neutrophils, according to the expression of CD101 on the neutrophils.

In some examples, the neutrophils are from a population of cells obtained from a human subject, and wherein the method further comprises detecting expression of CD10 on the neutrophils and separating the neutrophils into neutrophil subtypes according to the expression of CD10 on the neutrophils, wherein pre-neutrophils are CD10⁻CD101⁻, immature neutrophils are CD10⁻CD101⁺, and mature neutrophils are CD10⁺CD101⁺.

In some examples, the method further comprises detecting expression on the neutrophils and separating the neutrophils into neutrophil subtypes according to one or more biomarkers selected from a group comprising CD49d, CD16 and CXCR2, wherein pre-neutrophils are CD49d⁺CXCR2⁻, immature neutrophils are CD16⁻CXCR2⁻ and mature neutrophils are CD16⁺CXCR2⁺.

In some examples, the method comprises detecting expression of CD10 and CD101 with antibodies adapted to target CD10 and/or CD101.

In some examples, the neutrophils are from a population of cells obtained from a murine subject, and wherein pre-neutrophils and immature neutrophils are CD101⁻, and mature neutrophils are CD101⁺.

In some examples, the method further comprises detecting expression on the neutrophils and separating the neutrophils into neutrophil subtypes according to one or more biomarkers selected from a group comprising CXCR4 and ckit, wherein pre-neutrophils are CXCR4^{hi}ckit^{int}, immature neutrophils are CXCR4^{lo}ckit^{lo}, and mature neutrophils are CXCR4⁻ckit⁻.

In some examples, the neutrophils are from a population of cells obtained from a bone marrow, spleen and/or blood of the subject.

In some examples, there is provided a kit for separating neutrophils, the kit comprising: an agent for detecting the expression of CD101 on the neutrophils; and a separator for separating the neutrophils into neutrophil subtypes comprising pre-neutrophils, immature neutrophils and mature neutrophils according to the expression of CD101 on the neutrophils.

In some examples, the kit is for separating human neutrophils and the kit further comprises an agent for detecting the expression of CD10 on the human neutrophils, and the separator is adapted to separate the neutrophils into neutrophil subtypes according to the expression of CD10 on the neutrophils, wherein pre-neutrophils are CD10⁻CD101⁻, immature neutrophils are CD10⁻CD101⁺, and mature neutrophils are CD10⁺CD101⁺. In some examples, the agent for detecting the expression of CD10 is an antibody adapted to target CD10, and/or wherein the agent for detecting the expression of CD101 is an antibody adapted to target CD101.

In some examples, the kit further comprises an agent for detecting the expression on the neutrophils, of one or more biomarkers selected from a group comprising CD49d, CD16 and CXCR2, and wherein the separator is adapted to separate the neutrophils into neutrophil subtypes according to the expression of CD49d, CD16 and/or CXCR2 on the neutrophils.

In some examples, the kit is for separating murine neutrophils, and wherein the separator is adapted to separate the neutrophils into neutrophil subtypes according to the expression of CD101, wherein pre-neutrophils and immature neutrophils are CD101⁻, and mature neutrophils are CD101⁺.

In some examples, the agent for detecting the expression of CD101 is an antibody adapted to target CD101. In some examples, the kit further comprises an agent for detecting the expression on the neutrophils, of one or more biomarkers selected from a group comprising CXCR2, Ly6G, ckit, CD11b and CXCR4, and wherein the separator is adapted to separate the neutrophils into neutrophil subtypes according to the expression of CXCR2, Ly6G, ckit, CD11b and/or CXCR4 on the neutrophils.

In some examples, there is provided a method of isolating and/or enriching neutrophil subtypes, the method comprising: (a) detecting expression of CD101 on neutrophils in a population of cells; and (b) categorizing the neutrophils into neutrophil subtypes comprising pre-neutrophils, immature neutrophils and mature neutrophils according to the expression of CD101 on the neutrophils; and (c) isolating and/or enriching one or more desired neutrophil subtypes.

In some examples, the population of cells are obtained from a human subject, and wherein the method further comprises detecting expression of CD10 on the neutrophils and categorizing the neutrophils into neutrophil subtypes according to the expression of CD10 on the neutrophils, wherein pre-neutrophils are CD10⁻CD101⁻, immature neutrophils are CD10⁻CD101⁺, and mature neutrophils are CD10⁺CD101⁺.

In some examples, the method comprises detecting expression of CD10 and CD101 with antibodies adapted to target CD10 and/or CD101. More preferably, isolating one or more desired neutrophil subtypes comprises immobilizing the one or more desired neutrophil subtypes via the antibodies adapted to target CD10 and/or CD101.

In some examples, the method further comprises the step of validating the desired neutrophil subtype by detecting the expression of one or more biomarkers selected from a group comprising CD34, CD15, CD66b, CD49d, CD16, CXCR2 and Siglec8 (or SiglecF).

In some examples, the population of cells are obtained from a murine subject, and wherein pre-neutrophils and immature neutrophils are CD101⁻, and mature neutrophils are CD101⁺.

In some examples, the method comprises detecting expression of CD101 with antibodies adapted to target CD101. More preferably, isolating one or more desired neutrophil subtypes comprising immobilizing the one or more desired neutrophil subtypes via the antibodies adapted to target CD101.

In some examples, the method further comprising the step of validating the desired neutrophil subtype by detecting the expression of one or more biomarkers selected from a group comprising CXCR2, Ly6G, ckit, CD11b and CXCR4.

In some examples, the method comprising obtaining the population of cells from a bone marrow, spleen and/or blood of the subject.

In some examples, the method comprises administering the subject with Plerixafor, granulocyte-colony stimulating factor (G-CSF) and/or interleukin 3 (IL-3) prior to obtaining the population of cells from the subject.

In some examples, the desired neutrophil subtype is pre-neutrophils. More preferably, the method further comprises the step of expanding the pre-neutrophils with one or more growth factors selected from a group comprising interleukin 6 (IL-6), leukaemia inhibitory factor (LIF), stem cell factor (SCF), G-CSF and IL-3.

In some examples, there is provided a composition comprising pre-neutrophils, wherein the pre-neutrophils are CD10⁻CD101⁻.

In some examples, the pre-neutrophils are CD10⁻CD101⁻CD34⁻ CD15⁺CD66b⁺CD49d^{hi}Siglec8⁻ (or SiglecF⁻). In some examples, there is provided a composition comprising a therapeutically effective amount of pre-neutrophils for use in treatment, wherein the pre-neutrophils are CD10⁻CD101⁻.

In some examples, the composition is for use in the treatment of immunodeficiency related diseases and/or disorders in a patient. In some examples, the immunodeficiency related diseases and/or disorders are associated with cancer and/or infection. Even more preferably, the patient is immunocompromised.

In some examples, there is a composition comprising a therapeutically effective amount of pre-neutrophils for enhancing the immune system of a subject and/or maintaining an immune response in the subject, wherein the pre-neutrophils are CD10⁻CD101⁻.

In some examples, there is provided a use of pre-neutrophils in the manufacture of a medicament for treating immunodeficiency related diseases and/or disorders in a patient, wherein the pre-neutrophils are CD10⁻CD101⁻.

In some examples, the immunodeficiency related disease and/or disorders are associated with cancer and/or infection. More preferably, the patient is immunocompromised.

In some examples, there is provided a method of treating immunodeficiency related diseases and/or disorders in a patient, the method comprising administering to a therapeutically effective amount of pre-neutrophils to a patient, wherein the pre-neutrophils are CD10⁻CD101⁻.

In some examples, the immunodeficiency related disease and/or disorders are associated with cancer and/or infection. More preferably, the patient is immunocompromised.

In some examples, the method comprises administering a therapeutically effective amount of pre-neutrophils to the patient every three (3) to five (5) days.

In some examples, there is provided a method of enhancing the immune system of a patient, the method comprising the steps of: (a) obtaining a population of cells comprising neutrophils; (b) detecting expression of CD10 and CD101 on the neutrophils; (c) isolating pre-neutrophils from the population of cells, wherein the pre-neutrophils are CD10⁻CD101⁻; and (d) administering a therapeutically effective amount of the pre-neutrophils to the patient.

In some examples, step (b) comprises detecting expression of CD10 and CD101 with antibodies adapted to target CD10 and/or CD101.

In some examples, the method further comprises the step of expanding the pre-neutrophils prior to step (d). In some examples, the pre-neutrophils are expanded with one or more growth factors selected from a group comprising interleukin 6 (IL-6), leukaemia inhibitory factor (LIF), stem cell factor (SCF), G-CSF and IL-3.

In some examples, step (a) comprises obtaining a population of cells comprising neutrophils from the patient, preferably from the bone marrow of the patient. In some examples, the population of cells comprising neutrophils are obtained from cord blood.

In some examples, there is provided a method for diagnosing or prognosing a medical condition in a patient, the method comprising the steps of: (a) testing a sample comprising neutrophils obtained from a patient, to detect the expression of CD10 and CD101 on the neutrophils; (b) measuring the levels of pre-neutrophils, immature neutrophils and/or mature neutrophils in the sample, wherein pre-neutrophils are CD10⁻CD101⁻, immature neutrophils are CD10⁻CD101⁺, and mature neutrophils are CD10⁺CD101⁺: and (c) comparing the levels of the pre-neutrophils, immature neutrophils and/or mature neutrophils in the sample, to reference levels in a control to determine the absence or presence of the medical condition, or to predict the course of the medical condition.

In some examples, step (a) comprises detecting expression of CD10 and CD101 with antibodies adapted to target CD10 and/or CD101. In some examples, the method is an in vitro method.

In some examples, the sample is a bone marrow sample and/or a spleen sample, and wherein a level of pre-neutrophils in the sample higher than the reference level in the control indicates that the patient has an inflammatory medical condition. In some examples, the inflammatory medical condition is associated with an autoimmune disease, sepsis and/or cancer.

In some examples, the medical condition is a disease and the sample is a tissue sample, and wherein a level of immature neutrophils in the sample higher than the reference level in the control indicates that the patient has the disease. In some examples, the level of immature neutrophils correlates with the progression of the disease. In some examples, the tissue sample is a blood sample or a tumor sample, and wherein the disease is cancer. In some examples, the cancer is pancreatic cancer.

In some examples, there is provided a kit for detecting and/or predicting inflammation in a patient, the kit comprising: an agent for detecting the expression of CD10 on neutrophils and an agent for detecting the expression of CD101 on neutrophils to measure the level of pre-neutrophils in a sample taken from the patient, wherein the pre-neutrophils are CD10⁻CD101⁺; and a reference level for comparing the measured level of pre-neutrophils, wherein a level of pre-neutrophils in the sample higher than the reference level indicates that the patient has an inflammatory medical condition.

In some examples, there is provided a kit for diagnosis and/or prognosing cancer in a patient, the kit comprising: an agent for detecting the expression of CD10 on neutrophils and an agent for detecting the expression of CD101 on neutrophils to measure the level of immature neutrophils in a sample taken from the patient, wherein the immature neutrophils are CD10⁻CD101⁺; and a reference level for comparing the measured level of immature neutrophils, wherein a level of immature neutrophils in the sample higher than the reference level indicates that the patient has cancer, and/or wherein the level of immature neutrophils correlates with the progression of cancer.

### Experimental Section

Non-limiting examples of the present disclosure will be further described, which should not be construed as in any limiting the scope of the disclosure.

Experimental Model and Subject Details

Mice

Six to ten-week-old C57BL/6 mice were bred and maintained under specific pathogen-free (SPF) conditions in the Biological Resource Centre (BRC) of A*STAR, Singapore. Both males and females were used for experiments, but animals were sex- and age-matched in each experiment as much as possible. S100a8^{cre} (B6.Cg-Tg(S100A8-cre,-EGFP)1llw/J), Lyz2^{cre/cre} (B6.129P2-Lyz2^{tm1(cre)lfo}/J), Rosa26^{mT/mG} (STOCK Gt(ROSA)^{26Sortm4(ACTB-tdTomato,-EGFP)Luo}/J, Cxcl12^{DsRed/+} (STOCK Cxcl12^{tm2.1Sjm}/J), Rosa26^{LsL-YFP} (B6.129X1-Gt(ROSA)26Sor^{tm1(EYFP)Cos}/J), Albino mice (B6(Cg)-Tyr^{c-2J}/J), CD45.1 (B6.SJL-Ptprc^{a} Pepc ^{b}/BoyJ) and Cxcr4^{fl/fl} (B6.129P2-Cxcr4^{tm2Yzo}/J) mice were obtained from The Jackson Laboratory. For fate-mapping experiments, S100a8^{cre} and Lyz2^{cre/cre} mice were crossbread in-house with Rosa26^{LsL-YFP} and Rosa26^{mT/mG} mice respectively. Fucci-S/G2/M (#474) and the double transgenic Fucci-G1 (#639) mice were obtained from the RIKEN BioResource Center (Ibaraki, Japan; (Tomura et al., 2013)). Lyz2^{gfp/+} (Lyz2^{tm1.1Graf}) were provided by T. Graf (Centre for Genomic Regulation, Barcelona, Spain; (Faust et al., 2000)). Gain-of-function Cxcr4^{1013/+} (termed Cxcr4^{WHIM}) mice were provided by F. Bachelerie (INSERM 996, Clamart, France; (Balabanian et al., 2012)). Cebpe^{-/-} mice were provided by P. Koeffler (Cancer Science Institute of Singapore, NUS, Singapore) (Yamanaka et al., 1997). To generate C/EBP_{ε}-deficient chimeras, C57BL/6 mice were lethally irradiated (1100 rad) and reconstituted with Cebpe^{-/-} bone marrow cells alone, or with an equal proportion of WT CD45.1 bone marrow cells. S100a8^{cre} mice were crossbred in-house with Cxcr4^{fl/fl} to generate progeny with CXCR4-deficient neutrophils. For niche localization of neutrophil subsets, Fucci-S/G2/M (#474) mice were crossbred in-house with Cxcl12^{DsRed/+} mice. All transgenic mice were maintained on a C57BL/6 background and experiments were performed under the approval of the Institutional Animal Care and Use Committee (IACUC), in accordance with the guidelines of the Agri-Food and Veterinary Authority (AVA) and the National Advisory Committee for Laboratory Animal Research (NACLAR) of Singapore.

Human Blood, Bone Marrow and Cord Blood Samples

All samples were obtained in accordance with a favorable ethical opinion from SingHealth CIRB or A*STAR, the Singapore Immunology Network. Consent for bone marrow samples was sought from healthy donors who were already giving bone marrow for a different study or a medical cause. Cord blood units that do not meet clinical grade were obtained from the Singapore Cord Blood bank for research.

Method Details

Treatments

For 5-Fluorouracil (5-FU) myeloablative treatment, mice were injected once intraperitoneally with 150 mg/kg 5-FU (Sigma-Aldrich) or PBS control. For G-CSF treatment, mice were injected once intraperitoneally with 1.5 μg of G-CSF/anti-G-CSF antibody complex (G-CSFcx) as previously described (Rubinstein et al., 2013). Briefly, G-CSFcx were generated by incubating G-CSF (Neupogen) and anti-G-CSF (BVD11-37G10; Southern-Biotech) at 1:5 cytokine to antibody ratio for 20 min at 37° C. and were next diluted at least 10-fold in PBS before injection.

Tissue Preparation and Data Analysis for Flow Cytometry and Cell Sorting

Blood was obtained via an incision in the submandibular region and was then lysed in red blood cell lysis buffer (eBioscience). For BM cells, mice femurs were flushed using a 23-gauge needle in PBS containing 2 mM EDTA and 2% fetal bovine serum (FBS) and passed through a 70-μm nylon mesh sieve. Spleens were harvested and homogenized into single-cell suspensions using 70-μm nylon mesh sieves and syringe plungers. Antibodies were purchased from BD, Biolegend, eBioscience or R&D. For the identification of mouse myeloid cells, cells were stained with fluorophore-conjugated anti-mouse antibodies against CCR2 (475301), CD11b (M1/70), CD11c (N418), CD16/32 (2.4G2), CD31 (390), CD45 (30-F11), CD45.1 (A20), CD45.2 (104), CD49f (GoH3), CD62L (MEL-14), CD101 (Moushi101), CD115 (AFS598), cKit (2B8), CXCR2 (SA044G4), CXCR4 (2B11), CX3CR1 (SA011F11), F4/80 (BM8), Gr1 (RB6-8C5), I-A/I-E (M5/114.15.2), Ly6C (HK1.4), Ly6G (1A8) and Siglec-F (E50-2440), together with exclusion lineage markers that include CD3e (145-2C11), CD90.2 (53-2.1), B220 (RA3-6B2), NK.1.1 (PK136), and Sca-1 (D7). After exclusion of cell doublets and dead cells with DAPI, preNeu were identified as (Lin,CD115,Siglec-F)⁻ Gr1⁺CD11b⁺CXCR4^{hi}ckit^{int}CXCR2⁻, immature Neu were identified as (Lin,CD115,Siglec-F)⁻Gr1⁺CD11b⁺CXCR4^{lo}cKit^{lo}CXCR2⁻ and mature Neu were identified as (Lin,CD115,Siglec-F)⁻Gr1⁺CD11b⁺CXCR4⁻cKit⁻Ly6G⁺CXCR2⁺.

For identification of HSCs and HPCs, cells were stained with CD16/32 (2.4G2), CD34 (RAM34), CD48 (HM48-1), CD150 (TC15-12F12.2), cKit (268), Flt3 (A2F10), Ly6C (HK1.4) and Sca-1 (D7), together with exclusion lineage markers that include CD3e (145-2C11), CD11b (M1/70), CD90.2 (53-2.1), B220 (RA3-662), Gr1 (RB6-8C5) and NK.1.1 (PK136). After exclusion of cell doublets and dead cells with DAPI, LT-HSC were identified as Lin⁻cKit⁺Sca-1⁺CD150⁺CD48⁺, ST-HSC were identified as Lin⁻cKit⁺Sca-1⁺CD150⁻CD48⁻, MPP were identified as Lin⁻cKit⁺Sca-1⁺CD150⁻CD48⁺, CMP were identified as Lin⁻cKit⁺Sca-1⁻CD16/32^{int}CD34^{int}, GMP were identified as Lin⁻cKit⁺Sca-1⁻CD16/32^{hi}CD34^{hi}, MDP were identified as Lin⁻cKit⁺Sca-1⁻ CD115⁺Flt3⁺Ly6C⁻ and cMoP were identified as Lin⁻cKit⁺Sca-1⁻CD115⁺Flt3⁻Ly6C⁺. Flow cytometry acquisition was performed on a 5-laser BD LSR II (BD) using FACSDiva software, and data was subsequently analyzed with FlowJo software (Tree Star). Cell numbers were quantified with count beads (CountBright; Life Technologies) according to the manufacturer's instructions. Sorting of BM neutrophil subsets were performed using a BD ARIAII (BD) to achieve >98% purity.

Mass Cytometry (CyTOF) Sample Preparation, Acquisition and Analysis

For mass cytometry analysis, purified antibodies were obtained from BD Biosciences, Biolegend, eBioscience, BioXCell, and conjugated using MAXPAR® DN3 antibody labeling kits (Fluidigm) according to manufacturer's instructions. Mice were injected once intraperitoneally with 2 mg IdU (Sigma-Aldrich). Mice were euthanized 2 h later, femurs were harvested, flushed in PBS and passed through a 70-μm nylon mesh sieve. BM cells were plated in a 96-well round bottom plate at a density of 5×10⁶ cells per well. For human BM, aspirates were incubated in RPMI containing 10% FCS and 50 μM IdU for 1 h at 37° C. Cells were stained for viability with 100 μL of 50 μM of cisplatin (Sigma-Aldrich) for 5 minutes at 4° C. Cells were then washed with staining buffer (4% FBS, 0.05% sodium azide, 2 mM EDTA in 1× PBS) and incubated with anti-CCR2-APC, anti-CD34-FITC, anti-CD115-PE and anti-Flt3-biotin (mouse panel) or CXCR4-biotin, CXCR2-FITC, CD101-APC (human panel) in 50 μL reaction volume for 90 minutes at 4° C. Red blood cells were lysed with 1× RBS lysis buffer (eBioscience) and cells were washed with staining buffer. Cells were stained with 50 μL of metal isotope-labeled surface antibodies (See Table 1 and 2) on ice. After 30 minutes, cells were washed twice with staining buffer, once with PBS, and then fixed in 2% paraformaldehyde (PFA) (Electron Microscopy Sciences) in PBS at 4° C. overnight. The next day, cells were pelleted and re-suspended in 200 μL 1× permeabilization buffer (Biolegend) and allowed to stand for 5 minutes on ice. Cells were then washed once with PBS and incubated with cellular barcodes on ice for 30 minutes as previously described (Becher et al., 2014). Subsequently, cells were washed once with perm buffer and in staining buffer for 10 minutes on ice. Cellular DNA was labeled at room temperature with 250 nM iridium intercalator (Fluidigm) in 2% PFA/PBS. After 20 minutes, cells were washed twice with staining buffer.

Prior to acquisition, cells were washed twice with water before final re-suspension in water. Cells were pooled from all samples, enumerated, filtered and diluted to a final concentration of 0.6×10⁶ cells/mL. Mass-tag barcoding was used so that all samples could be acquired simultaneously. EQ Four Element Calibration Beads (Fluidigm) were added to the pooled samples at a final concentration of 1% prior to acquisition. Samples were acquired on a CyTOF2 (Fluidigm) equipped with a Super Sampler fluidic system (Victorian Airship & Scientific Apparatus LLC) at an event rate of <500 events per second. After mass cytometry acquisition, data were exported in flow-cytometry (FCS) format, normalized and events with parameters having zero values were randomized using a uniform distribution of values between minus-one and zero. Each sample containing a unique combination of two metal barcodes was de-convoluted by Boolean gating using FlowJo software (Tree Star). Subsequently, manual gating was done to exclude residual beads, debris and dead cells. BM CD45⁺IdU⁺ (proliferative cells) and CD45⁺IdU⁻ (non-proliferative cells) were gated using Flowjo, and exported as a FCS file. Random subsampling without replacement was performed to select 90000 events. Dimensional reduction of the CyTOF data was performed selecting the markers listed in Table 1 by t-distributed stochastic neighbor embedding (t-SNE) using the Cytofkit R package (Chen et al., 2016; van der Maaten and Hinton, 2008). Clusters were generated using the FIowSOM implementation in Cytofkit. Median intensity values per cluster for each marker were calculated and exported to produce heatmaps using R. The identity of each cluster was inferred based on the expression of each individual marker.

TABLE 1
Mouse cyToF panel, related to STAR methods

| Metal | Antibody | Clone | Cat number | Company |
|---|---|---|---|---|
| 89 | CD45 | 30-F11 | 3089005B | Fluidigm |
| 112/114 | CD19 | 6D5 | Q10379 | Invitrogen |
| 115 | CD90 | T24/31 | BE0212 | BioXCell |
| 127 | IdU | | I7125 | Sigma-Aldrich |
| 141 | CD43 | S7 | 553268 | BD Biosciences |
| 142 | MHCII | Y-3P | BE0178 | BioXCell |
| 143 | B220 | RA3.3A1/6.1 | BE0067 | BioXCell |
| 144 | CD11a | FD441.8 | BE0005-1 | BioXCell |
| 145 | Gr-1 | RB6-8C5 | 108402 | Biolegend |
| 146 | CD88 | 20/70 | 135802 | Biolegend |
| 147 | Ly6G | 1A8 | 127602 | Biolegend |
| 148 | Ly6c | HK1.4 | 128002 | Biolegend |
| 149 | CD31 | MEC13.3 | 102502 | Biolegend |
| 150 | CX3CR1 | SA011F11 | 149002 | Biolegend |
| 151 | CD62L | MEL-14 | 104402 | Biolegend |
| 152 | CD11c | N418 | 117302 | Biolegend |
| 153 | CD11b | M1/70 | 101202 | Biolegend |
| 154 | CD49b | DX5 | 108902 | Biolegend |
| 155 | cKit | 2B8 | 105829 | Biolegend |
| 156 | BST2 | 120G8 | N/A | Purified |
| | | | | in house |
| 157 | CXCR2 | SA044G4 | 149302 | Biolegend |
| 158 | TER119 | TER-119 | 116202 | Biolegend |
| 159 | F4/80 | CI:A3-1 | MCA497GA | Bio-Rad |
| 160 | Flt3 Biotin | A2F10 | 135308 | Biolegend |
| | (Primary) | | | |
| | Streptavidin | | | Purified |
| | (Secondary) | | | in house |
| 161 | CD34 FITC | RAM34 | 553733 | BD Biosciences |
| | (Primary) | | | |
| | anti-FITC | FIT-22 | 408302 | Biolegend |
| | (Secondary) | | | |
| 162 | PD-L1 | 10F.9G2 | 124302 | Biolegend |
| 163 | CD150 | TC15-12F12.2 | 115933 | Biolegend |
| 164 | NK1.1 | PK136 | 108743 | Biolegend |
| 165 | Ly6B.2 | 7/4 | NBP2- | Novus |
| | | | 13077AF488 | Biologicals |
| 166 | CD48 | HM48-1 | 103402 | Biolegend |
| 167 | CXCR4 | L276F12 | 146502 | Biolegend |
| 168 | CCR2 APC | 475301 | FAB5538A | R&D Systems |
| | (Primary) | | | |
| | anti-APC | APC003 | 408002 | Biolegend |
| | (Secondary) | | | |
| 169 | CD115 PE | AFS598 | 61-1152-82 | Thermo Fisher |
| | (Primary) | | | |
| | anti-PE | PE001 | 408102 | Biolegend |
| | (Secondary) | | | |
| 170 | CD49f | GoH3 | 313602 | Biolegend |
| 171 | FceR1 | MAR-1 | 14-5898-82 | Thermo Fisher |
| 172 | Sca-1 | D7 | 108102 | Biolegend |
| 173 | CD49d | R1-2 | 553154 | BD Biosciences |
| 174 | CD24 | M1/69 | 101802 | Biolegend |
| 175 | Siglec-F | E50-2440 | 552125 | BD Biosciences |
| 176 | CD16/32 | 2.4G2 | 553140 | BD Biosciences |

TABLE 2
Human cyToF panel, related to STAR methods

| Metal | Antibody | Clone | Cat number | Company |
|---|---|---|---|---|
| 89 | CD45 | HI30 | 3089003B | Fluidigm |
| 113 | CD15 | HI98 | 301902 | Biolegend |
| 115 | CD57 | HCD57 | 322302 | Biolegend |
| 140 | CD2 | RPA-2.10 | 300202 | Biolegend |
| 141 | CD13 | WM15 | 301702 | Biolegend |
| 142 | CD5 | UCHT2 | 300602 | Biolegend |
| 143 | CD62L | DREG-56 | 555541 | BD Biosciences |
| 144 | CD38 | HIT2 | 303502 | Biolegend |
| 145 | CD45RA | HI100 | 304102 | Biolegend |
| 146 | CD3 | UCHT1 | 300402 | Biolegend |
| 147 | HLA-DR | L243 | 307602 | Biolegend |
| 148 | CD66b | G10F5 | 555723 | BD Biosciences |
| 149 | CD10 | HI10a | 312202 | Biolegend |
| 150 | CD235ab | HIR2 | 306602 | Biolegend |
| 151 | CD7 | M-T701 | 555359 | BD Biosciences |
| 152 | Siglec 8 | 7C9 | 347102 | Biolegend |
| 153 | FcER1 | AER-37 | 16-5899-82 | eBioscience |
| | | (CRA1) | | |
| 154 | CCR3 | 5E8 | 310702 | Biolegend |
| 155* | CD123 | 6H6 | 306002 | Biolegend |
| 156 | CD14 | M5E2 | 301802 | Biolegend |
| 157* | CD31 | WM59 | 303102 | Biolegend |
| 158 | CD56 | NCAM16.2 | 559043 | BD Biosciences |
| 159 | CD33 | WM53 | 303402 | Biolegend |
| 160 | CXCR4 Biotin | 12G5 | 306504 | Biolegend |
| | (Primary) | | | |
| | Streptavidin | | | Synthesized |
| | (Secondary) | | | in house |
| 161* | CXCR2 FITC | 5E8/CXCR2 | 320704 | Biolegend |
| | (Primary) | | | |
| | anti-FITC | FIT-22 | 408302 | Biolegend |
| | (Secondary) | | | |
| 162* | CD88 | S5/1 | 344302 | Biolegend |
| 163* | CD66a/c/e | ASL-32 | 342302 | Biolegend |
| 164 | CD116 | 4H1 | 305902 | Biolegend |
| 165 | CD303 | 201A | 354202 | Biolegend |
| 166 | CD117 | 104D2 | 313202 | Biolegend |
| 167 | CD49d | 9F10 | 304302 | Biolegend |
| 168 | CD101 APC | BB27 | 331007 | Biolegend |
| | (Primary) | | | |
| | anti-APC | APC003 | 408002 | Biolegend |
| | (Secondary) | | | |
| 169 | CD49f | GoH3 | 313602 | Biolegend |
| 170 | CD64 | 10.1 | 305002 | Biolegend |
| 171 | CD34 | 581 | 343502 | Biolegend |
| 172 | CD44 | IM7 | 103002 | Biolegend |
| 173* | CD19 | HIB19 | 302202 | Biolegend |
| | CD20 | 2H7 | 302302 | Biolegend |
| 174 | CX3CR1 | K0124E1 | 355702 | Biolegend |
| 175 | CD11c | B-ly6 | 555390 | BD Biosciences |
| 176 | CD11b | ICRF44 | 301312 | Biolegend |
| 209 | CD16 | 3G8 | 3209002B | Fluidigm |

Whole-Mount Tissue Preparation and Immunostaining

Freshly dissected femurs of 6-10-week-old mice were fixed in 4% PFA in 1× PBS containing 30% sucrose for 3 hours at room temperature with gentle shaking. The bones were washed with 1× PBS for 3 times (30-minute interval). Femurs were next placed in decalcifying solution containing 10% Ethylenediamine tetra-acetic acid (EDTA) in PBS at pH=7 for 2 days at 4° C. After 3 washes with 1× PBS of 30 minutes each, femurs were embedded in 4% agarose and sectioned using a vibratome (Leica VT1000S) at a thickness of 250 μm. Femur sections were blocked and permeabilized in staining buffer containing 10% dimethyl sulphoxide (DMSO) and 2.5% goat and donkey serum overnight. Sections were stained for 3 days with rat anti-mouse S100A9 (21310, Abcam) and rabbit polyclonal laminin 1+2 (ab7363, Abcam) in staining buffer. Sections were subsequently washed 3 times with 1× PBS (1-hour interval), and stained for 2 days with anti-rat AF555 IgG and anti-rabbit AF647 IgG (Life Technologies). Sections were washed 3 times in 1× PBS (1-hour interval), and placed in RapiClear 1.55 (Sunjin Lab) for at least 30 min for refractive index matching. Sections were finally mounted in RapiClear 1.55 between two coverslips and sealed with vacuum grease (Dow Corning).

Multi-Photon Image Acquisition of Femur Sections

Three-dimensional (3D) mosaic images of femur sections were acquired using a LaVision TriM Scope II microscope (LaVision BioTec), equipped with a water dipping objective (20× magnification, 1.0 NA, 2 mm WD; XLUMPLFLN20xW, Olympus) and a Chameleon-pulsed infrared laser (titanium sapphire; Coherent). Acquisitions were performed two excitation wavelengths: 990 nm and 800 nm. 990 nm excitation was used for the simultaneous imaging of Fucci-(S-G2-M) positive cells (λₑₘ=505 nm), second harmonic generation (SHG) (λₑₘ=495 nm), AF555 (λₑₘ=565 nm) and DsRed (λₑₘ=580 nm). Subsequently, imaging was performed at 800 nm for the acquisition of AF647 (λₑₘ=670 nm). Filter used were: 494/41, 525/50, 565/40, 620/60 and 665/40 (Semrock). Dichroic mirrors used were 495LP, 560LP, 620LP, 591sh (Semrock) and 640LP (Chroma Technology). Images were acquired with the following settings: 450 μm×450 μm, 517×517 pixels, 600 Hz line scan with 2 frames of line averaging, using a 2 μm z-step size with a depth of 250 μm. The distal epiphysis was chosen as the area for imaging to maintain consistency between samples. 3D mosaic Z-stack images were stitched together using FIJI is just ImageJ (FIJI), and subsequently rendered and analyzed using Imaris software (Bitplane). Spectral spillover between AF555 and DsRed was removed using Imaris with the channel arithmetic plugin. S100A9⁺Fucci-(S-G2-M)⁺ and S100A9⁺Fucci-(S-G2-M)⁻ cells were identified using the spots function tool in Imaris. Calculation of distance to the nearest vessels and CAR cell was performed using the Distance Transform Matlab-based XTension built in Imaris. Raw statistics were then exported for further analysis in Prism (Graphpad).

Cytospin and Wright-Giemsa Staining

Sorted neutrophil subsets (1×10⁵ cells each) were spun onto glass slides using Cytospin 4 Cytocentrifuge (Thermo scientific), dried for 20 minutes, fixed in methanol and stained with the Hema 3 manual staining system (Fisher Diagnostics) according to the manufacturer's protocol. Images were acquired with an Olympus BX43 equipped with a 100× oil immersion objected, and image brightness was adjusted with Photoshop (Adobe).

Transcriptomics

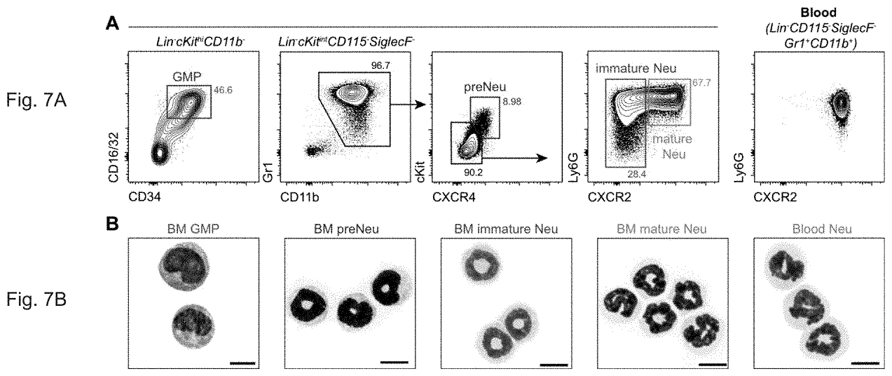

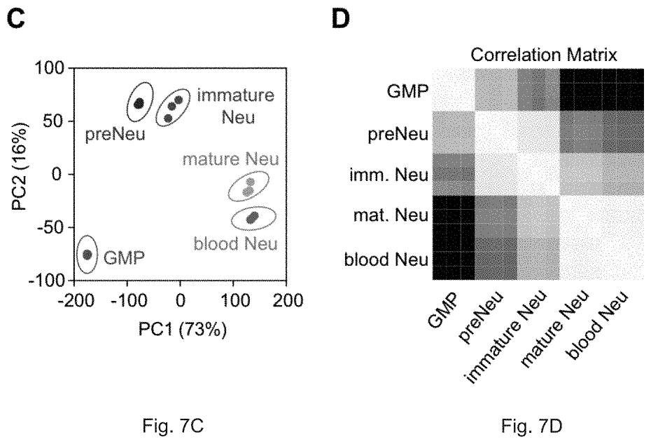

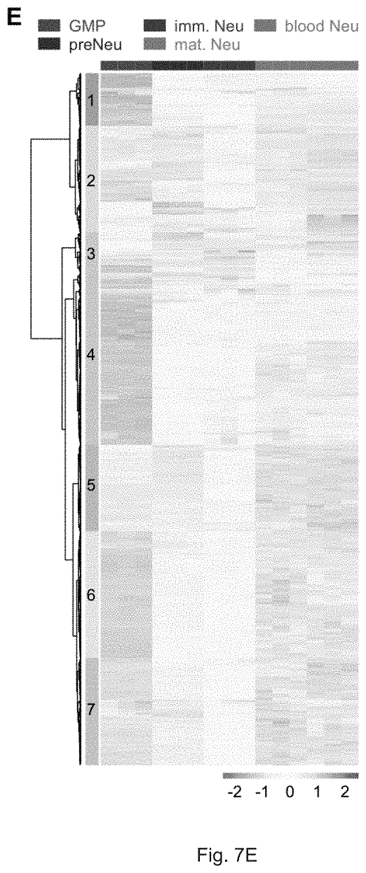

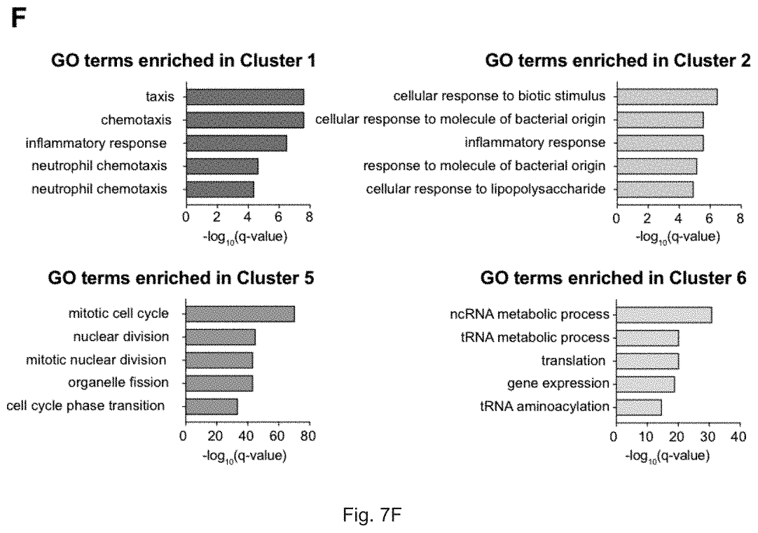

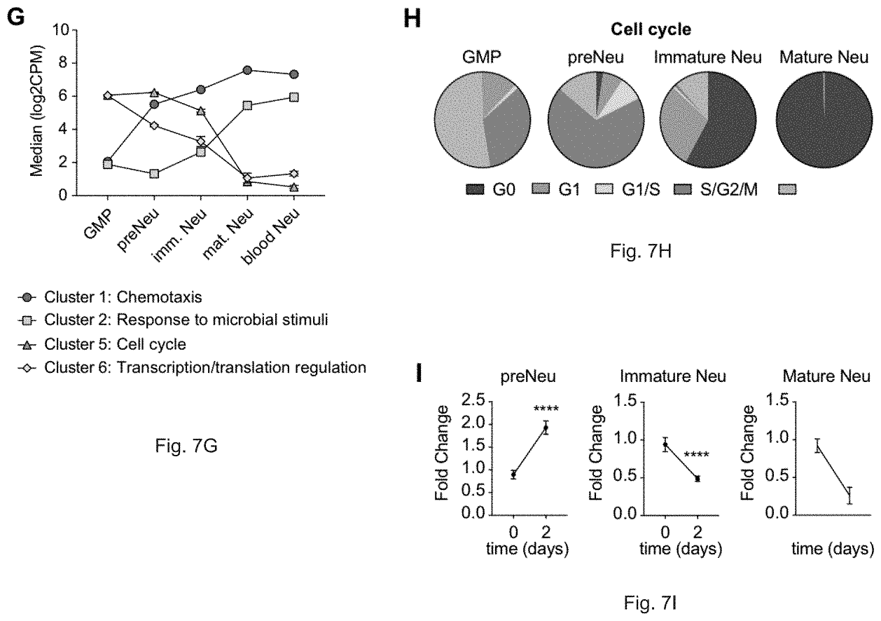

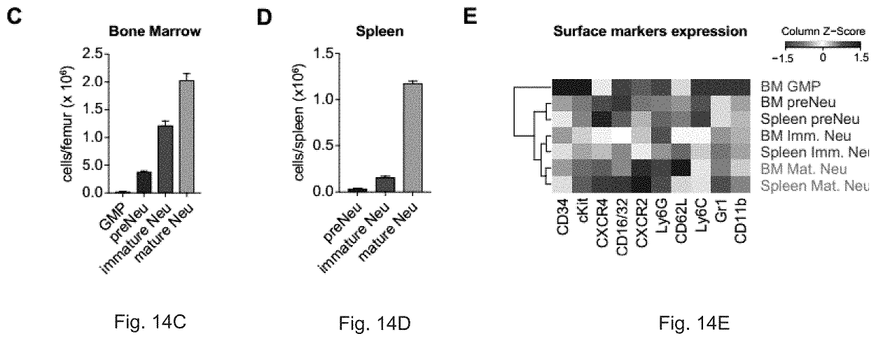

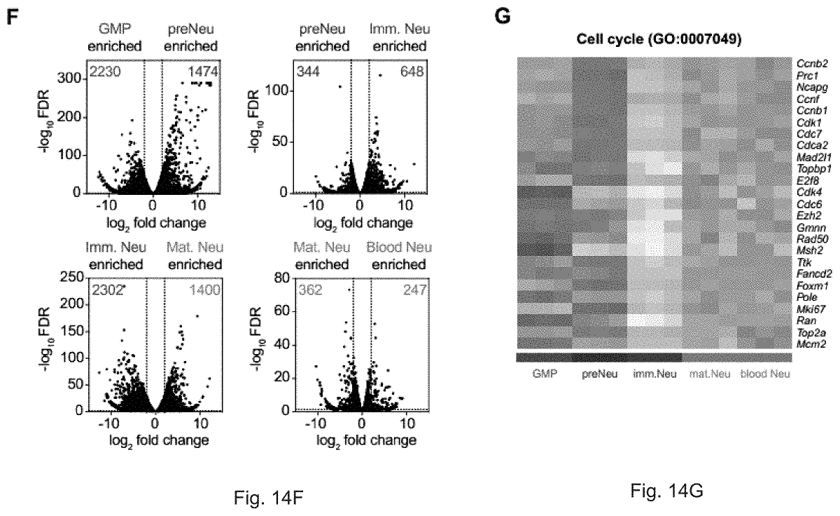

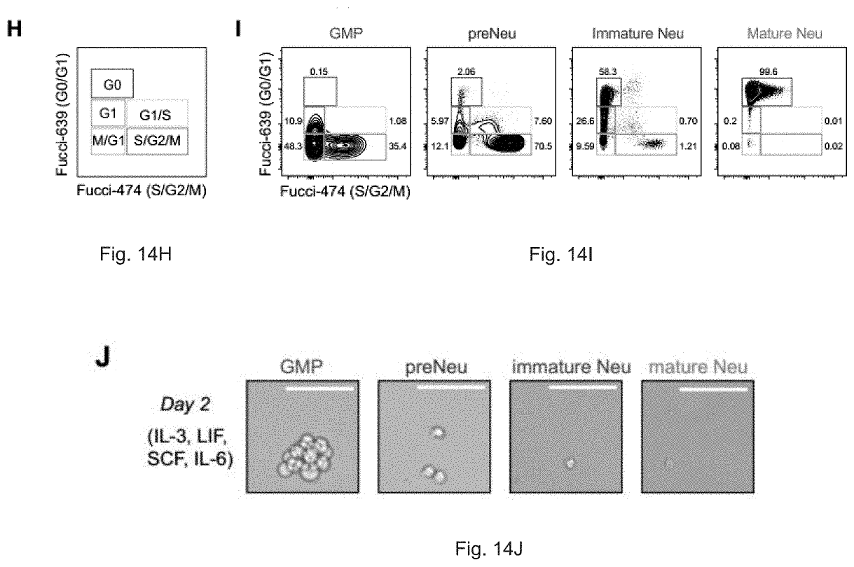

GMP, preNeu, immature Neu, mature Neu and blood Neu from 3 different mice were sorted based on the gating strategy depicted in FIGS. 7A and 14A. BM Transitional pre-monocytes (tpMo) and BM mature Ly6C^{hi} monocytes were sorted as Lin(CD3,CD90.2,B220,NK1.1,Ly6G)⁻CD115⁺Flt3⁻Ly6C⁺CXCR4^{hi}CD11b^{lo} and Lin(CD3,CD90.2,B220,NK1.1,Ly6G)⁻CD115⁺Flt3⁻Ly6C⁺CXCR4^{lo}CD11b^{hi} respectively from 3 different mice (see gating strategy in (Chong et al., 2016)). Total RNA isolation was subsequently performed using Arcturus PicoPure RNA Isolation kit according to the manufacturer's protocol. All mouse RNAs were analyzed on Perkin Elmer Labchip GX system for quality assessment with RIN>7.7. cDNA libraries were prepared using 2 ng of total RNA and 1 μL of a 1:50000 dilution of ERCC RNA Spike in Controls (Ambion) using SMARTSeq v2 protocol (Picelli et al., 2014), except for the following modifications: (1) use of 20 μM TSO; and (2) use of 250 pg of cDNA with 1/5 reaction of Illumina Nextera XT kit. The length distribution of the cDNA libraries was monitored using DNA High Sensitivity Reagent kit on the Perkin Elmer Labchip. All 18 samples were subjected to an indexed PE sequencing run of 2×51 cycles on an Illumina HiSeq 2500 Rapid mode.

RNA-Seq data in the form of FASTQ files were subsequently mapped to the mouse genome build mm10 using the STAR alignment software. The mapped reads were then counted using featureCounts (part of Subread package) based on the GENCODE M7 annotations. The raw counts were then used for a differential gene expression analysis (DEG) using edgeR (R version 3.1.2) with FDR<0.05 and log₂FC>2 to identify genes differentially regulated in neutrophil subsets to generate volcano plots. Count per million reads (CPM) values were calculated from raw counts using edgeR (R version 3.1.2). The CPM values were then log₂-transformed in R (x→log₂(1+x)). For PCA, hierarchical clustering and correlation matrices, the gene expression matrix was first segregated using the top 20% variable genes (as measured by standard deviation across samples) and then those that were significantly associated with a cell population (FDR-corrected ANOVA, q-value <0.05) resulting in 4820 DEGs. For hierarchical clustering, Euclidean distance and the Ward aggregation criterion and the pheatmap package were used to plot the results as a heatmap. The correlation matrix was computed using Pearson's correlation coefficients. Gene ontology (GO) enrichment (GO Biological Process 2015) of DEGs was done using Enrichr (Chen et al., 2013).

Computational Inference of Developmental Path

R package seriation 2 (Hahsler et al., 2008) was used to find a suitable linear order for GMP, preNeu, immature Neu, mature Neu and blood Neu. Six different seriation methods including TSP, R2E, ARSA, HC, GW and OLO. TSP, ARSA, GW and OLO produced identical and the best results in terms of shortest path length, minimal AR events and minimum Moore stress. Seriation analysis was done using log₂CPM values of all detected genes.

In Vitro Cell Culture

Sorted cells (3×10⁴ for each neutrophil subset) were plated onto 96-well plates in triplicates and cultured at 37° C., 5% CO₂ in Iscove's Modified Dulbecco's Medium with 25 mM HEPES and L-Glutamine (Chemtron) containing 10% (vol/vol) FBS, 1 mM sodium pyruvate, penicillin (100 U/ml) and streptomycin (100 ug/ml). Colony-formation assays were performed as described before (Hettinger et al., 2013). Briefly, sorted cells (3×10⁴ for each neutrophil subset) were cultured for in Iscove's modified Dulbecco's medium (Sigma) with the supplements mentioned above, 1% (wt/vol) methylcellulose (MethoCult M3134, Stem Cell Technologies) and a combination of cytokines (50 ng/ml SCF, 20 ng/ml LIF, 10 ng/ml IL-3, 20 ng/ml IL-6). Representative colony images were collected with an Olympus IX-81 microscope (Olympus). Image brightness was adjusted with Photoshop.

BrdU Pulsing Assays

For in vivo assays, mice were injected intraperitoneally with 2 mg 5-bromo-2′-deoxyuridine (BrdU; Sigma-Aldrich) at indicated time points. To detect BrdU incorporation into neutrophil subsets, cells were stained with a fixable vitality dye (Zombie UV fixable viability kit; Biolegend), surface-stained, fixed, permeabilized, and subjected to intracellular staining with FITC-conjugated anti-BrdU antibody, according to the manufacturer's protocol (BrdU Flow kit; BD) before analysis by flow cytometry.

Adoptive Cell Transfer

Sorted Lyz2^{gfp/+} preNeu (2×10⁵ cells) were transferred intra-BM into wild-type recipients as described previously (Chong 2016). Briefly, recipient mice were anesthetized with ketamine (150 mg/kg)/xylazine (10 mg/kg), and had their right leg shaved to expose the kneecap. Sorted preNeu were resuspended in 1× PBS at a concentration of 2×10⁴ cells/μL, and a volume of 10 μL was administered into the tibia through the kneecap using a 29-gauge insulin needle. At 24 and 48 hours after cell transfer, tibias were collected, stained and analyzed by flow cytometry.

Laser-Induced Sterile Injury Model

Neutrophil subsets were sorted from either Lyz2^{gfp/+} (GFP) or Rosa26^{mT/mG} (tdTomato) transgenic mice as indicated, and were mixed in a 1:1 ratio (each 2.5×10⁵ cells). Cells were resuspended at a concentration of 0.1×10⁵ cells/μL. A 2.5 μL volume of neutrophil suspension was injected intradermally in the ear with a Hamilton syringe (33-gauge, 62RN). B6(Cg)-Tyrc^{-2J}/J (B6 albino) mice were used as recipient mice in all experiments. After two to three hours, mice were prepared for skin multiphoton imaging and laser focal injury was then performed as described previously (Li et al., 2012). Briefly, anesthetized mice were set up onto a custom ear imaging stage platform to stabilize the ear for intravital imaging. To induce a sterile injury, a chosen area (75 μm²) close to the injection site was briefly exposed to a focused laser pulse (850 nm) for ˜5 s. For image acquisition, an excitation wavelength of 990 nm was used to collect GFP (λₑₘ=510 nm), tdTomato (λₑₘ=580 nm) and second harmonic generation (SHG) (λₑₘ=495 nm) simultaneously. Filters used were 494/41, 510/20 and 579/34 (Semrock). Dichroic mirrors used were a 495 LP (Semrock), 560 LP (Semrock) and 640 LP (Chroma Technology). A scan-field dimension of 500 μm×500 μm, with a Z-step size of 4 μm was used to acquire the 40-50 μm stacks, taken at every half-minute intervals for 1 hour. Mice body temperatures were kept at 37° C. with a heating pad and mice ears were separately warmed at 35° C. during imaging. After acquisition, data correction and analysis were conducted using Imaris (Bitplane). Where necessary, FIJI is just ImageJ (FIJI) was used to correct for drifts that occurred during acquisition. Cell tracking was done semi-automatically in Imaris using the “spots” function and the “auto-regressive motion” algorithm. Reconstructed images and videos were finally generated using Imaris.

Oxidative Burst Assay

Sorted neutrophils (5×10⁵ for each cell subset) were incubated with 2.5 μg/mL Dihydrorhodamine 123 (DHR) (ThermoFisher) in RPMI, and subjected to 50 nM Phorbol 12-Myristate 13-Actetate (PMA) (Sigma-Aldrich) for 20 min at 37° C. Cells were subsequently washed with PBS and the fluorescence intensities of each subset were measured by flow cytometry.

Phagocytosis Assay In Vitro

DH5a Escherichia coli (E. coli) expressing GFP (Chua and Wong, 2013) were grown in Lysogeny Broth (LB) medium overnight at 37° C. to an Optical Density (OD) at 600 nm of 1.5-1.8, at which point the bacteria were diluted and grown for 1-2 hours to an OD600 of ˜0.5, and were finally washed twice with PBS. Sorted neutrophils (1×10⁵ for each cell subset) were incubated with bacteria in a ratio of 1:100 for 2 hours at 37° C. After incubation, the cells were washed with PBS, fixed with 2% PFA and analyzed by flow cytometry.

Reverse Passive Arthus (RPA) Reaction

RPA was conducted as described before (Li et al., 2016). Briefly, mice were intravenously injected with Evans blue dye (Sigma-Aldrich) at 8 μL/g bodyweight, 10 mg/ml in saline). RPA reaction is initiated by intradermal injection of 1.5 μL of 10 mg/mL anti-BSA (Sigma-Aldrich), followed by intraperitoneal injection of 200 μL of 5 mg/ml BSA (Sigma-Aldrich). For quantification of neutrophil numbers, mouse ears were subjected to tissue homogenization and enzymatic digestion as described (Li et al., 2016), followed by flow-cytometric analysis. For quantification of vascular leakage, readings were obtained through digital photographic analysis methods.

CLP-Induced Mid-Grade Sepsis

Cecal ligation and puncture was performed as described previously (Rittirsch et al., 2009). Briefly, the peritoneal cavity was exposed under ketamine/xylazine anesthesia and the cecum was exteriorized. 50% of the cecum was ligated distal of the ileo-cecal valve using a non-absorbable 7-0 suture. A 26-gauge needle was used to perforate the distal end of the cecum, and a small drop of feces was extruded through the puncture before being relocated into the peritoneal cavity. The peritoneum was closed and mice were subsequently treated with saline and Buprenorphine (5-20 mg/kg) by subcutaneous injection. For sham-operated controls, the peritoneum was exposed and the cecum was exteriorized before closing the peritoneum as mentioned above. Mice were euthanized and harvested 24 hours or 2 weeks after the surgery where indicated. For bacterial CFU measurements, blood and peritoneal fluid were collected after 24 hours and cultured overnight at 37° C. on blood-agar base plates (Trypticase Soy Agar II; Fisher scientific) and LB agar plates respectively.

Orthotopic Pancreas Tumor Model

Mice were administered intrapancreatic injections of FC1242 tumor cells (kind gift from Dr. Dannielle D. Engle, Tuveson lab) derived from Pdx1^{cre}; LsL-Kras^{G12D/+}; LsL-Trp53^{R172H/+} (termed KPC) mice as previously described (Zambirinis et al., 2015). Briefly, mice were anesthetized with ketamine/xylazine, and had their abdomen shaved and swabbed with antiseptics. A 5 mm vertical incision was made in the skin and abdominal layer at a point 1 cm down from the xiphoid process of the sternum, and 1 cm to the right of the midline. The pancreas was exposed, 1×10⁵ tumor cells were resuspended in 1× PBS and mixed with matrigel (BD) in a 1:1 ratio and were injected as a volume of 504 into the body of the pancreas to form a visible bolus using a 29-gauge insulin needle. The pancreas was then returned to the abdominal cavity. The abdominal layer was closed with absorbable 5/0 sutures, while the skin was closed with non-absorbable 5/0 sutures. Superglue was applied over the sutures to ensure that they do not come undone after surgery. Mice were resuscitated with saline and were subcutaneously administered Buprenophrine (10 mg/kg) and Enrofloxacin (Baytril, 1.5 mg/kg) for the 2 days following surgery. Mice were euthanized at day 27-30 following surgery and tumor weights were recorded.

Quantification and Statistical Analysis

Statistical analyses were done using Prism software (Graphpad). Student's t-test or one-way analysis of variance (ANOVA) with Bonferroni correction were performed. For correlation analysis, linear regression was used to generate the best-fit line for graphical representation, and Pearson's correlation test was performed to generate p values. P values <0.05 were considered as statistically significant.

Results

Multiparameter Analysis of Bone Marrow Cells Identifies Proliferating Neutrophils with Distinct Phenotypic Signatures.

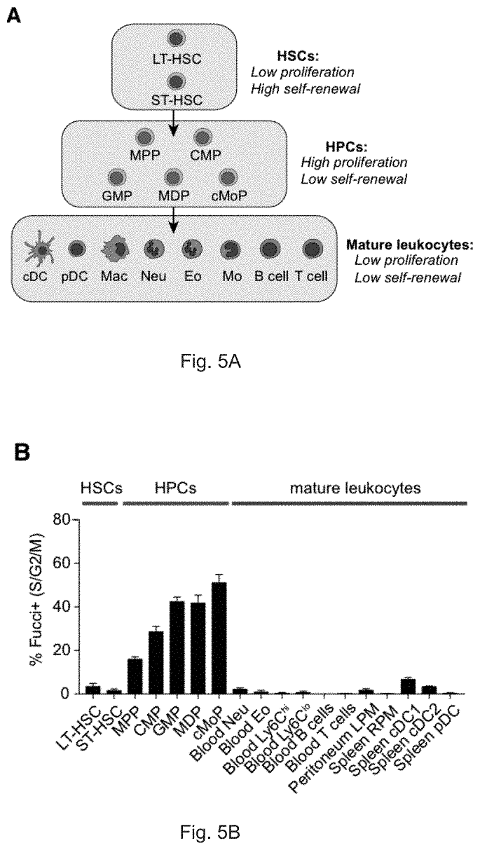

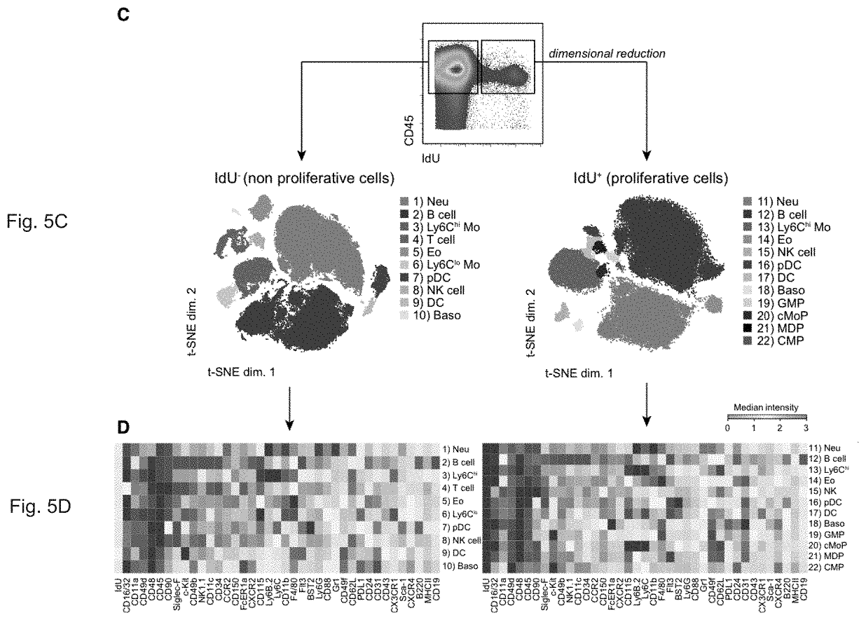

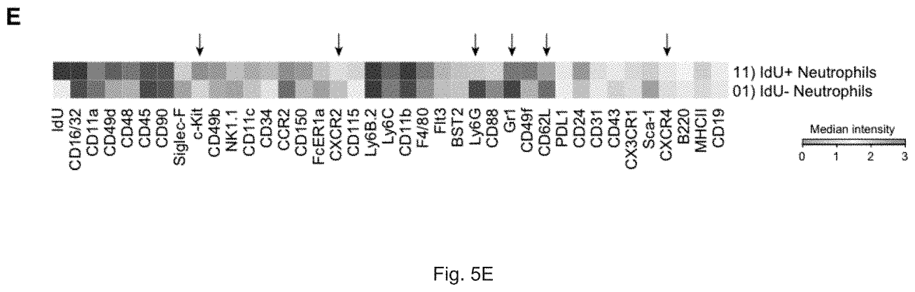

Cellular proliferation is central to hematopoiesis. The classical model suggests a hierarchical order, which begins with the cellular amplification of hematopoietic stem cells (HSCs) that leads to the generation of all blood cell lineages (FIG. 5A) (Manz and Boettcher, 2014; Orkin and Zon, 2008). Upon differentiation of slow proliferating HSCs to hematopoietic progenitor cells (HPCs), HPCs commit towards their respective cell lineages by reducing their self-renewal capacity and proliferate extensively instead to meet the demand of mature lineage specific cells (FIG. 5A). HPC differentiation to mature leukocytes represents a late stage of development for most immune cells and thus, mature leukocytes have little ability to self-renew or proliferate, with the exception of lymphocytes, DCs and tissue-resident macrophages (FIG. 5A) (Ginhoux and Jung, 2014; Manz and Boettcher, 2014).

To determine if this hematopoietic proliferative framework can be delineated experimentally, various immune cell types at different stages of development were analysed using the Fucci-474 reporter mouse that labels cells undergoing the S, G2 or M phase of the cell cycle (termed Fucci-(S-G2-M)) (Sakaue-Sawano et al., 2008; Tomura et al., 2013). It was found that less than 10% of HSCs and mature leukocytes were in cell cycle (FIG. 5B). In contrast, more than 40% of GMP engaged in cell proliferative activity (FIG. 5B), in agreement with previously published data (Yo et al., 2015).

To explore the phenotypic diversity between cycling leukocytes and those in cell cycle arrest, mass cytometry was utilized to segregate major leukocyte lineages in the BM (Becher et al., 2014) through 40 different expression markers. CD45⁺ hematopoietic cells were first separated into proliferative IdU⁺ and non-proliferative IdU⁻ cells (FIG. 5C). The t-distributed stochastic neighbor embedding (t-SNE) algorithm was next utilized to visualize similarities between cells on a 2D map (FIG. 5C) and Cytofkit was used to generate clusters (Chen et al., 2016; van der Maaten and Hinton, 2008).

Using this method, it was confirmed that HPCs such as CMPs and GMPs were highly proliferative and were present only among IdU⁺ cells. In addition, mature and terminally differentiated leukocytes were only present within the IdU⁻ populations. Notably, neutrophils formed the second largest cluster in both the proliferating and non-proliferating subsets (FIG. 5C, green). However, while B cell precursors, which forms the largest cluster among proliferative cells are well defined, the identification of a neutrophil committed precursor and their subsequent developmental stages remains unclear. Hence, the inventors extracted the median intensities of each marker and generated heatmaps for every identified cluster among the proliferating and non-proliferating populations (FIG. 5D). Differentially expressed markers among neutrophils was next explored by performing a side-by-side comparison of the markers expressed between IdU⁺ and IdU⁻ neutrophils (FIG. 5E). Using this approach, differentially expressed markers between proliferative and non-proliferative neutrophils that included cKit, CXCR2, Ly6G, Gr1, CD62L and CXCR4 were found. Of note, this approach was not only valid for neutrophils, but this approach was also able to identify differentially expressed markers between IdU⁺ and IdU⁻ basophils and eosinophils and Ly6C^{hi} monocytes (FIG. 12).

Collectively, the approach redefines the identity of neutrophil precursors by categorizing their maturation stages according to their proliferative and molecular properties.

Fucci-(S-G2-M) Reporter Mouse Reveals a Proliferative Neutrophil Precursor.

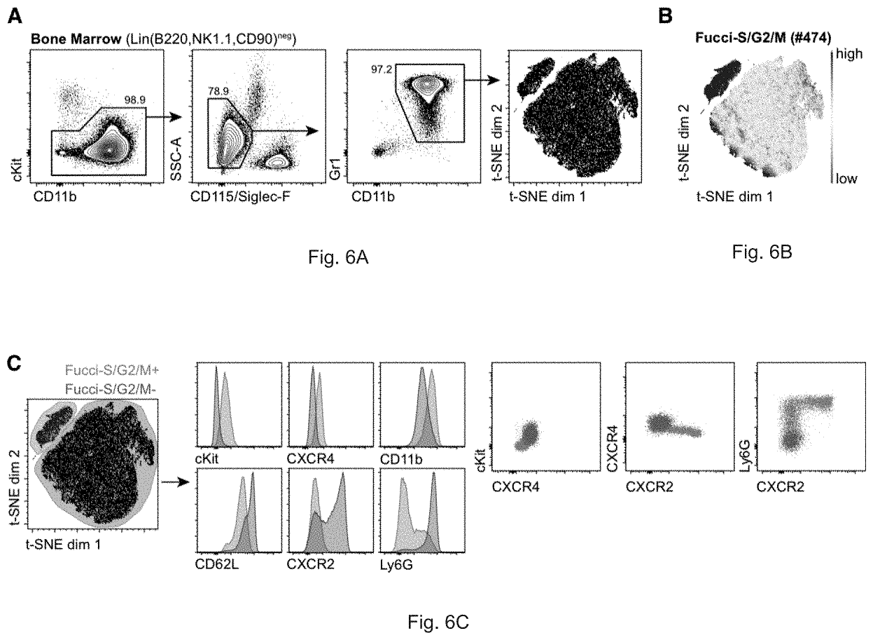

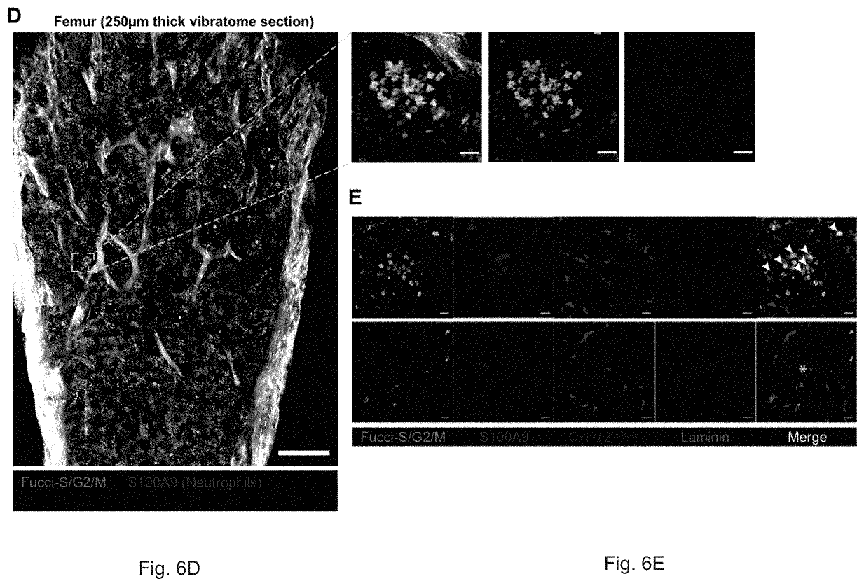

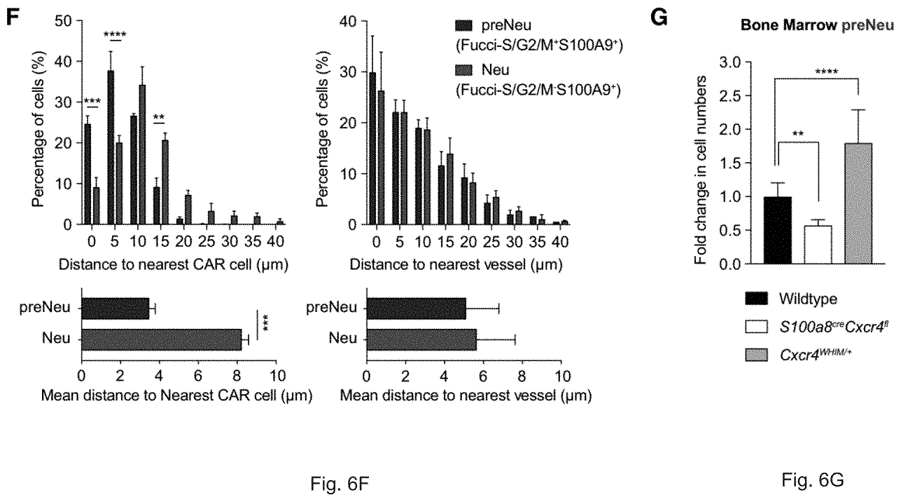

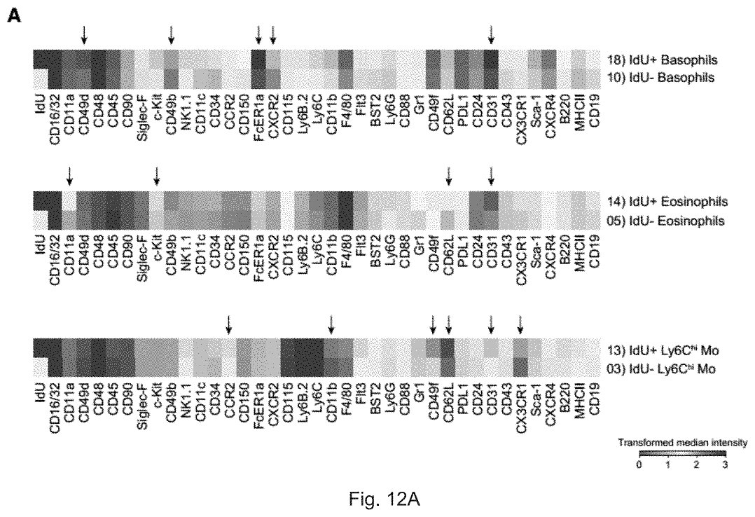

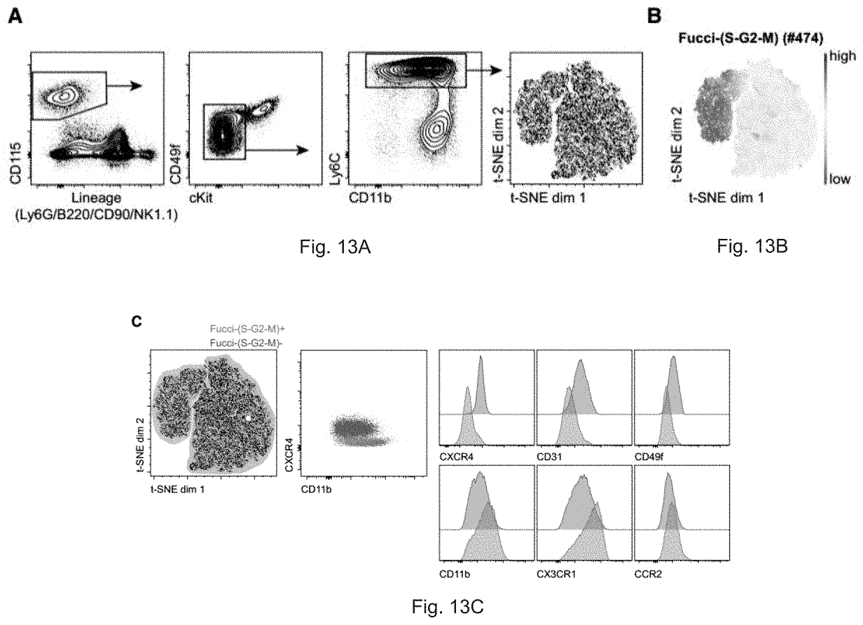

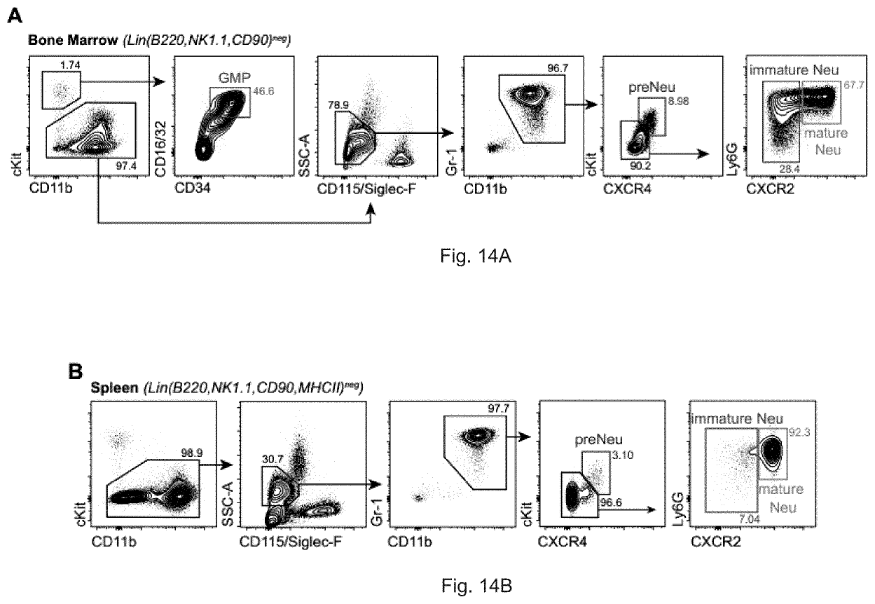

To identify a committed neutrophil progenitor or precursor, the markers identified in FIG. 5E and the Fucci-(S-G2-M) mouse were used. Lineage-positive cells, early progenitors (cKit^{hi} cells), monocytes (SSC^{lo}CD115⁺), eosinophils (SSC^{hi}SiglecF⁺) were excluded and Gr1⁺CD11b⁺ neutrophils (FIG. 6A) were gated. Dimensional reduction using t-SNE revealed two distinct clusters that were distinguishable based on Fucci-(S-G2-M) expression (FIG. 6B). The expression of various markers between proliferating (Fucci-(S-G2-M)⁺) and non-proliferating (Fucci-(S-G2-M)⁻) neutrophils was next compared. In agreement with the mass cytometry data (FIG. 5E), non-proliferating neutrophils highly expressed Ly6G and CXCR2, while proliferating neutrophils were Ly6G^{lo}CXCR2⁻ and were positive for cKit and CXCR4 (FIG. 6C). This “Fucci-based” approach proved to be robust as it identified proliferative transitional pre-monocytes (tpMo) among BM Ly6C^{hi} monocytes (FIG. 13), which the inventors have recently characterised (Chong et al., 2016).

Taken together, the cell cycle-based approaches have identified heterogeneity among the neutrophil lineage and revealed a putative proliferative neutrophil precursor, which the inventors term pre-neutrophils (preNeu).

PreNeu Form Clusters in Close Proximity with CXCL12-Abundant Reticular (CAR) Cells.

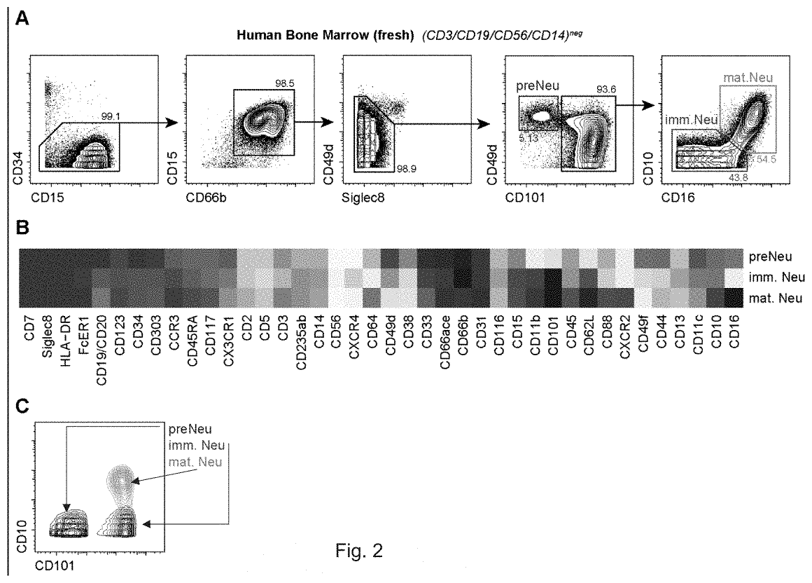

Hematopoietic lineage survival and development requires specialized BM niche factors to generate mature hematopoietic cells from HSCs and HPCs (Frenette et al., 2013). Since preNeu display proliferative activity (FIGS. 6B and 2C), the inventors next investigated if they were localized in a specialized niche.

Magnified femur areas (FIG. 6D) revealed that S100A9⁺Fucci-(S-G2-M)⁺ preNeu were preferentially found in clusters in vivo, consistent with their proliferative activity (FIG. 6D). Furthermore, preNeu were situated closely to CXCL12 chemokine-expressing cells (FIG. 6E). Since CAR cells and endothelial cells support the growth of HSCs and HPCs (Anthony and Link, 2014), the inventors next questioned whether preNeu were preferentially positioned in close proximity to these BM niche cells. Hence, the inventors quantified the distance between preNeu (S100A9⁺Fucci-(S-G2-M)⁺) or neutrophils (S100A9⁺Fucci-(S-G2-M)⁻) to the nearest CAR cell (Cxcl12-DsRed⁺) and endothelial cell (Laminin⁺). By doing so, it was found that neither preNeu nor neutrophils were specifically in contact with BM endothelial cells (FIGS. 6E and 6F). In contrast, it was found that the majority of preNeu, but not neutrophils, were positioned in clusters <5 μm away from CAR cells (FIGS. 6E and 6F). Since CAR cells produce large amounts of CXCL12, a neutrophil-specific CXCR4-deficient mouse (termed S100a8^{cre}Cxcr4^{fl}) was used. By doing so, the inventors detected a 50% decrease of BM preNeu in S100a8^{cre}Cxcr4^{fl} as compared to wildtype controls. Conversely, a CXCR4 gain-of-function mutation (termed Cxcr4^{WHIM}) showed an approximate 2-fold increase in BM preNeu as compared to wildtype counterparts (FIG. 6G).

In summary, the data indicates that proliferating preNeu cluster in close proximity to CAR cells and are retained in the BM through CXCR4.

Neutrophils Express Distinct Genetic Signatures Throughout Their Development.

While the cell cycle-based approaches have identified proliferative preNeu, it remains unclear how they may fit within the neutrophil lineage. To address this question, the Lin⁻Gr1⁺CD11b⁺ neutrophil fraction was analysed with cKit⁺CXCR4⁺ preNeu excluded (FIGS. 7A and 14A). While blood neutrophils were mostly Ly6G⁺ and CXCR2⁺, BM neutrophils were heterogeneous for these two markers, segregating them into Ly6G⁺CXCR2⁺ neutrophils that resemble blood neutrophils and a population of Ly6G^{lo/+}CXCR2⁻ neutrophils that appeared to be immature based on the lack of CXCR2 (FIG. 7A).

To better understand the developmental relationship between preNeu, immature neutrophils (Ly6G^{lo/+}CXCR2⁻; termed immature Neu) and mature neutrophils (Ly6G⁺ CXCR2⁺; termed mature Neu), these three subsets were sorted and their morphology were compared with sorted GMP and blood neutrophils. While GMP displayed a largely uncondensed nucleus with an immature cytosol, neutrophils progressively condensed their nucleus from a toroidal shape in preNeu to a poly-segmented shape in BM mature and blood neutrophils (FIG. 7B). PreNeu and immature Neu were also identified in the spleen (FIGS. 14B and 14E), but in much fewer numbers than the BM (FIGS. 14C and 14D) and these cells were absent from the blood (data not shown).

The inventors next determined how the molecular signature of these neutrophil precursors differed through whole transcriptome sequencing (RNAseq). Principal-component analysis (PCA) of all transcripts revealed distinct gene expression profiles between all subsets (FIG. 7C). Furthermore, while GMP, preNeu and immature Neu displayed distinct gene signatures, BM mature Neu and blood Neu displayed a similar gene expression profile using the Pearson correlation matrix (FIG. 7D). Although BM immature Neu and mature Neu were distinguishable only through CXCR2 expression based on the limited phenotypical analysis, these two subsets showed vast transcriptomic differences with more than 3000 differentially expressed genes (FIGS. 7D and 14F).

The 20% most variable genes were next plotted in a heatmap and seven distinct clusters of genes that were differentially regulated during neutrophil development were identified (FIG. 7E). Two gene clusters (1 and 2) were upregulated during neutrophil development, and comprised genes involved in chemotaxis, neutrophil motility (cluster 1) and response to microbial stimuli (cluster 2) (FIGS. 7F and 7G). In contrast, two gene clusters (5 and 6) were downregulated during neutrophil development, and consisted of genes involved in cell cycle and regulation of gene expression (FIGS. 7F and 7G). Since the RNAseq analysis revealed a progressive decrease in the expression of cell cycle-associated genes during neutrophil development, the inventors next determined the precise point where they lost their proliferative capacity in their lineage development. Using a dual Fucci reporting system 474 (S-G2-M)/639 (G0-G1) (Tomura et al., 2013), it was found that preNeu showed the highest amount of cells in the S phase while immature Neu abruptly arrested cell cycle and progressively entered the G0 phase upon maturation into mature Neu (FIGS. 7H and 14H). In agreement with these results, a downregulation of cell cycle-related genes between GMP to mature Neu, including Mki67, Cdk1, and Top2a (FIG. 14G) was found. Having established that these neutrophil subsets are engaged in different stages of the cell cycle, it was next investigated how these differences could translate to biological function. To address this question, in vitro culture of preNeu, immature and mature Neu was performed for two days. While immature and mature neutrophil numbers rapidly declined in culture, it was found that preNeu could expand in culture (FIGS. 7H and 7I). Colony forming assays also revealed that preNeu divided but did not form colonies unlike GMP, while immature and mature neutrophils did not divide at all (FIG. 14J). Taken together, the inventors have characterised three discrete subsets of BM neutrophils that are phenotypically, morphologically and transcriptionally distinct.

PreNeu are Committed Towards the Neutrophil Lineage.

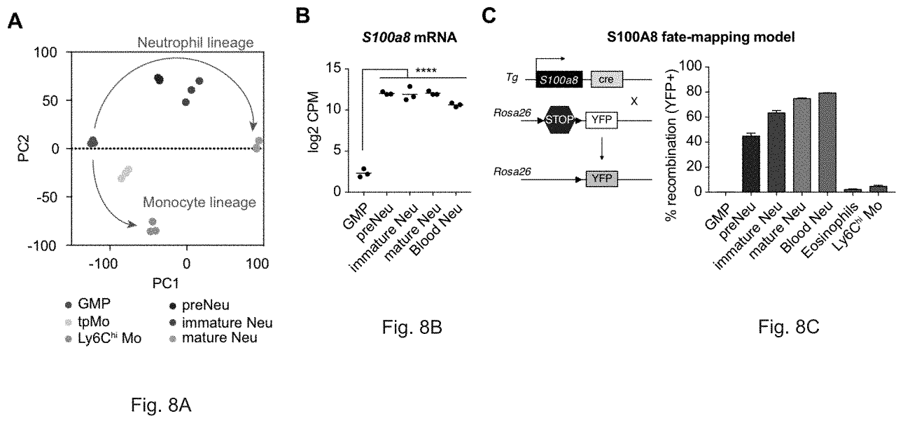

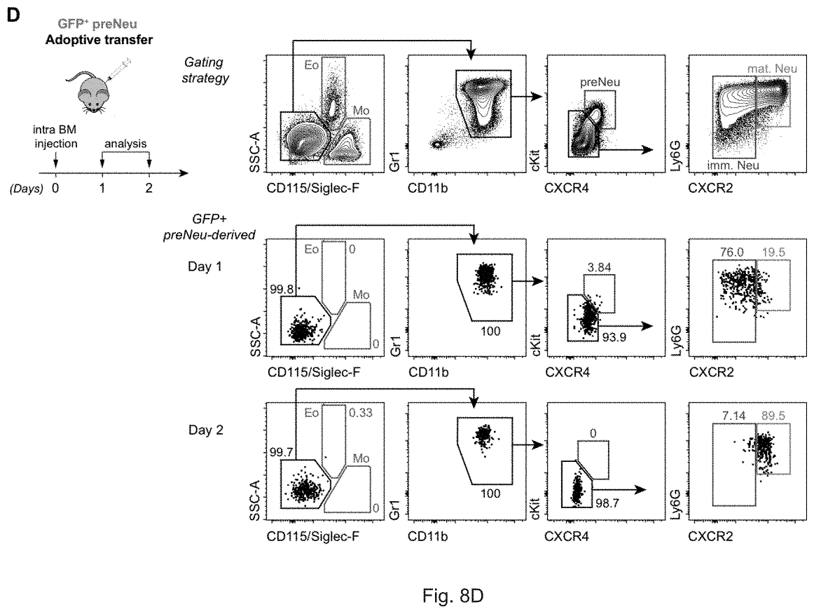

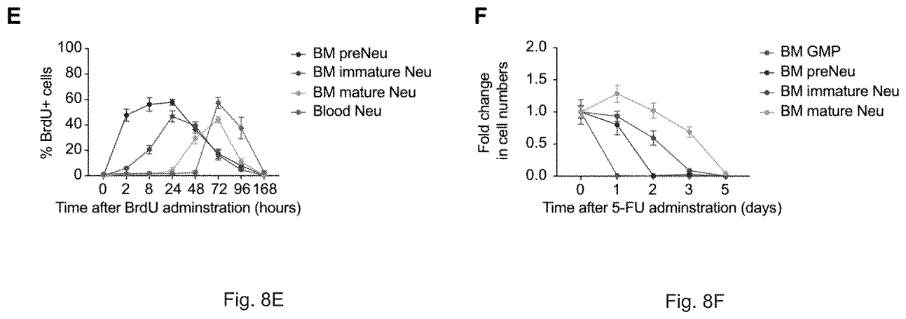

While the current results suggested a maturation process from preNeu into immature Neu and mature Neu, it remained unclear whether preNeu were fully committed towards the neutrophil lineage. To address this question, the inventors first compared the transcriptomic signature of members of the neutrophil and monocyte lineages together with GMP (FIG. 8A). PCA of all transcripts revealed that preNeu were more similar to members of the neutrophil lineage, such as immature Neu, than they were to members of the monocyte lineage (FIG. 8A).

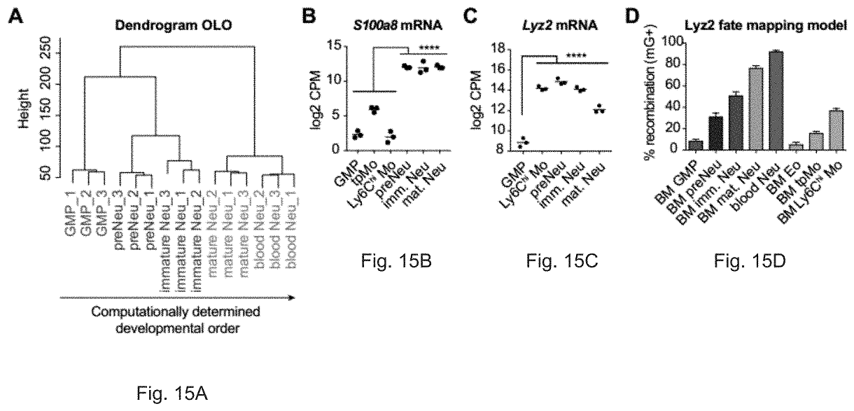

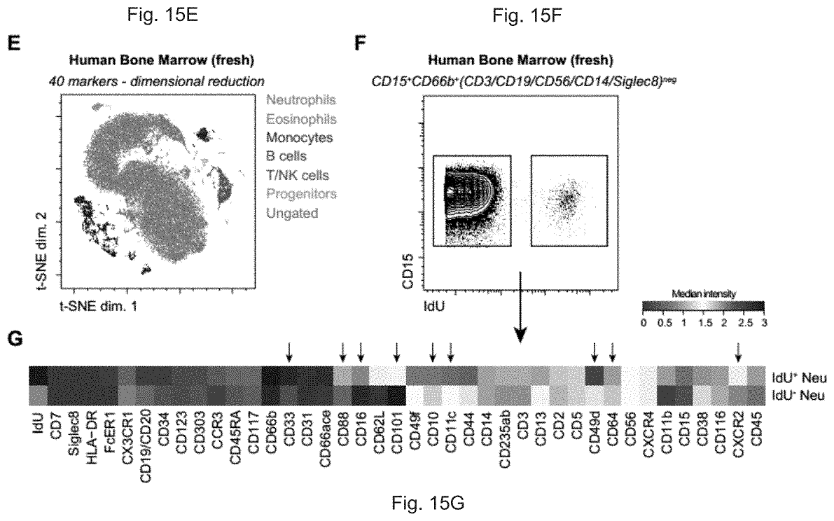

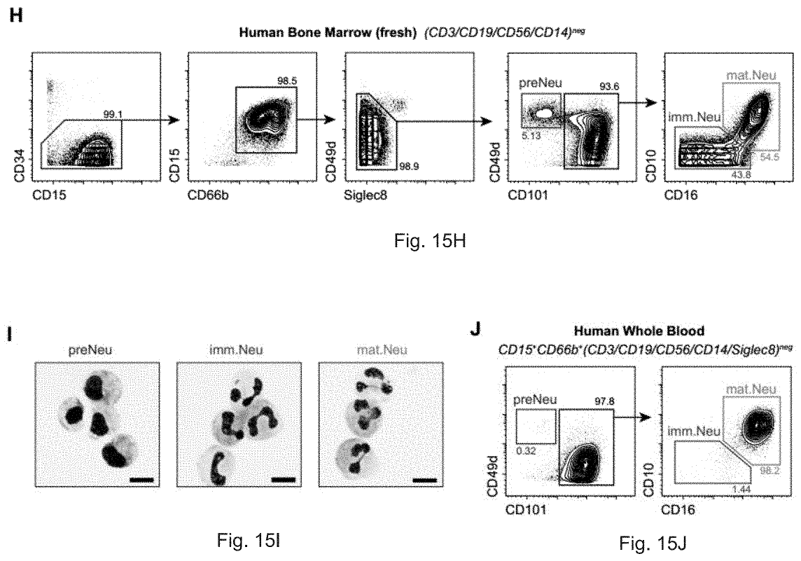

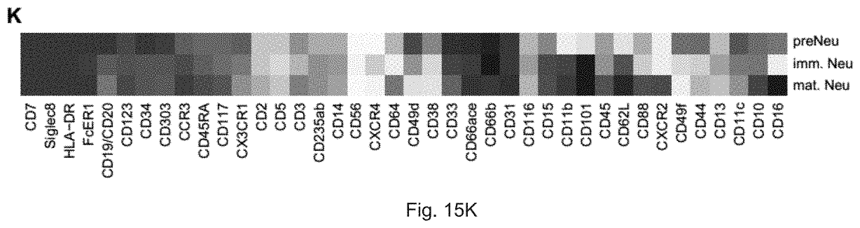

To further understand the developmental continuum of neutrophils subsets, gene expression data from these subpopulations was used and a hierarchical clustering by optimal leaf ordering (OLO) was performed. Dendrogram obtained through this method determined a development order from GMP to preNeu, immature Neu, mature Neu and finally to blood neutrophils (FIG. 15A).

The inventors validated this result by devising a cre-based fate-mapping strategy to follow the development of preNeu and their progeny. The inventors based their strategy on the expression of S100a8 as this gene was found to be selectively upregulated only from the preNeu stage, but minimally expressed in GMP and cells from the monocyte lineage, consistent with previously published data (FIGS. 8B and 15B) (Passegue et al., 2004; Reber et al., 2017). S100a8^{cre} mice were crossed together with the Rosa26^{LsL-YFP} and the recombination rate was determined during the development of myeloid cells. While GMP showed no detectable recombination, preNeu exhibited ˜40% recombination rate that progressively increased in immature, mature and blood neutrophils to reach ˜80% recombination (FIG. 8C). Similar results were found using a Lyz2^{cre}-based strategy (FIGS. 15C and 15D). In contrast, other myeloid cells such as monocytes and eosinophils showed <10% recombination rates (FIG. 8C). Together, these results suggest that preNeu only give rise to immature and mature neutrophils.

These results were next validated in vivo by transferring GFP⁺ preNeu into the BM of wildtype recipients, which gave rise to immature Neu after one day and differentiated into mature Neu one day later (FIG. 8D). Importantly, preNeu did not give rise to monocytes, eosinophils or other cell lineages, which further confirms the fate-mapping analyses (FIG. 8D).

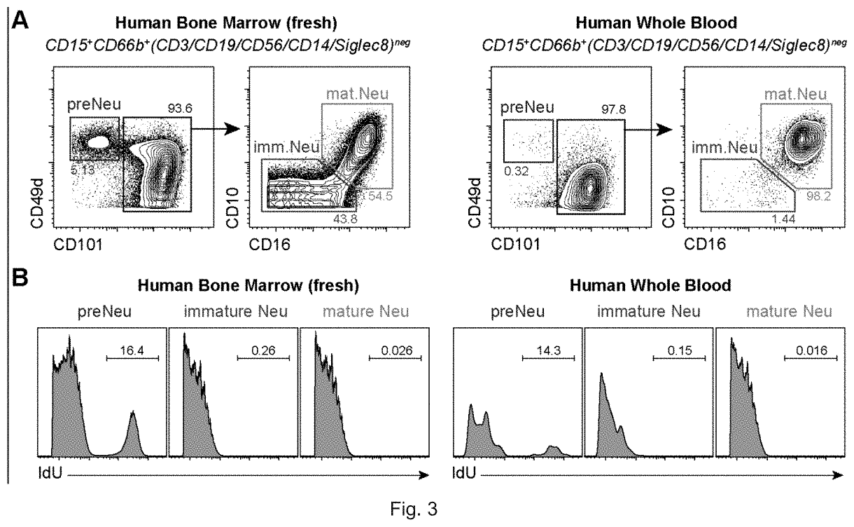

To further establish the developmental timeline of preNeu in vivo, their intrinsic highly proliferative capacity was employed as a marker (FIG. 3H and S3H). 5-bromo-2′-deoxyuridine (BrdU) that is only incorporated into actively dividing cells such as preNeu was employed, thereby allowing their differentiation over time to be followed as previously used for monocytes (Yona et al., 2013). As expected, only preNeu were BrdU⁺ 2 h after administration (FIG. 8E). Using this method, it was found that preNeu progressively differentiated into immature Neu after 24 h, mature Neu after 48 h and finally egressed into the circulation after 72 h (FIG. 8E). In addition, treatment with the chemotherapeutic drug 5-fluorouracil (5-FU) that inhibits thymidine synthesis and causes dividing cells to undergo apoptosis, triggered a successive loss of GMP, preNeu, immature and mature Neu (FIG. 8F).

Furthermore, the presence of preNeu in humans was confirmed by employing a similar workflow performed in FIG. 5C. To detect a putative neutrophil precursor in human BM (FIG. 15E), CD15⁺CD66b⁺ total neutrophils were manually gated and differentially expressed markers between proliferative (IdU⁺) and non-proliferative (IdU⁻) neutrophils including CD10, CD16, CD49d and CD101 (FIGS. 15F and 15G) were identified. Using this strategy, the human equivalents of preNeu, immature and mature Neu (FIG. 15H-K) were identified. Akin to mice, preNeu and immature Neu were virtually absent from the blood, thereby validating the workflow (FIG. 15J).

Together, the results provide evidence to show that preNeu acts as a proliferative precursor of the neutrophil lineage in both mice and humans.

The development of preNeu to mature Neu is accompanied by functional changes associated with maturation.

Thus far, the data suggested a developmental process that occurs from preNeu to immature Neu and finally to mature Neu. To gain further insights into the functional processes that occur during this maturation, the gene expression of transcriptions factors (TFs) involved at different stages of myeloid cell development (FIG. 9A) was analyzed. As anticipated, multipotent GMP highly expressed Cebpa, which is necessary for granulopoiesis initiation, as well as TFs involved in the development of other myeloid lineages such as Irf8, Gata1 and Gata2 (Fiedler and Brunner, 2012; Yanez et al., 2015). In contrast, preNeu highly expressed Gfi1 and Cebpe, two TFs crucial for early neutrophil differentiation (Hock et al., 2003; Yamanaka et al., 1997). Finally, mature and circulating neutrophils showed high expression of Cebpd and Spi1 (PU.1), which is in line with their role in terminal granulopoiesis (Borregaard, 2010) (FIG. 9A).

TFs from the C/EBP family promote the expression of granule associated enzymes. Specifically, C/EBPα induces the expression of primary granule enzymes (such as Mpo) (Ford et al., 1996), while C/EBP_{ε} and C/EBPδ promote secondary (such as Ltf) and tertiary granules enzymes (such as Mmp8) respectively (Gombart et al., 2003). Since a highly-coordinated expression of these TFs across the neutrophil lineage (FIG. 9A) was observed, it was next determined whether this pattern was correlated with granule expression. Indeed, while primary granules were mainly expressed at the GMP stage, secondary granules were formed mostly within preNeu and immature Neu, and tertiary granules were associated with mature Neu, thereby matching the expression patterns of C/EBPα, C/EBP_{ε} and C/EBPδ (FIGS. 9A and 9B).

The functional differences that occur across the different stages of differentiation was next determined. To this end, key genes involved in reactive oxygen species (ROS) production, phagocytosis and chemotaxis were looked at (FIGS. 9C, 9F and 9G). The highest expression of these genes was found among mature Neu (FIGS. 9C, 9F and 9G). This was associated with a superior capacity of mature Neu to produce ROS upon phorbol myristate acetate (PMA) stimulation (FIG. 9D), and to phagocytose bacteria as compared to the other populations of the neutrophil lineage (FIG. 9E). In addition, it was found that preNeu had a reduced migratory capacity, as mature Neu quickly swarmed towards the necrotic core while preNeu were immotile in response to sterile injury (FIG. 9H).

Altogether, the inventors found a progressive functional maturation of the neutrophil lineage during development with mature Neu possessing the full range of neutrophil effector functions.

C/EBP_{ε}-deficiency impairs the development of preNeu and downstream neutrophil populations.

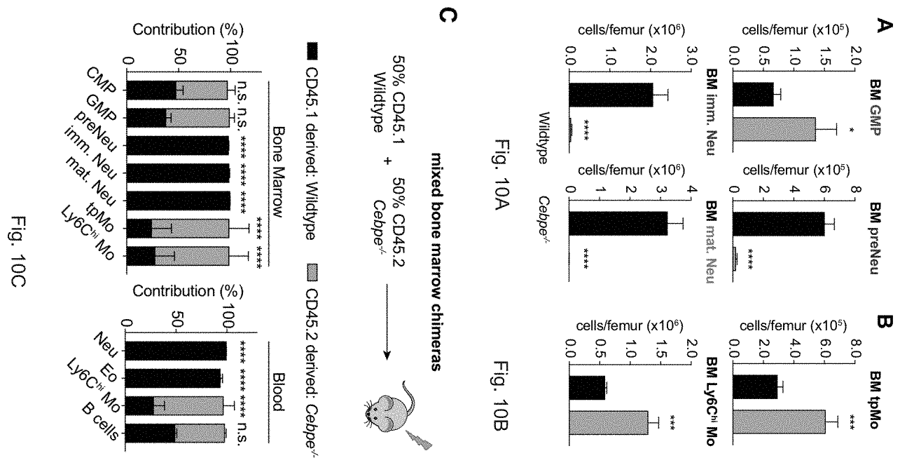

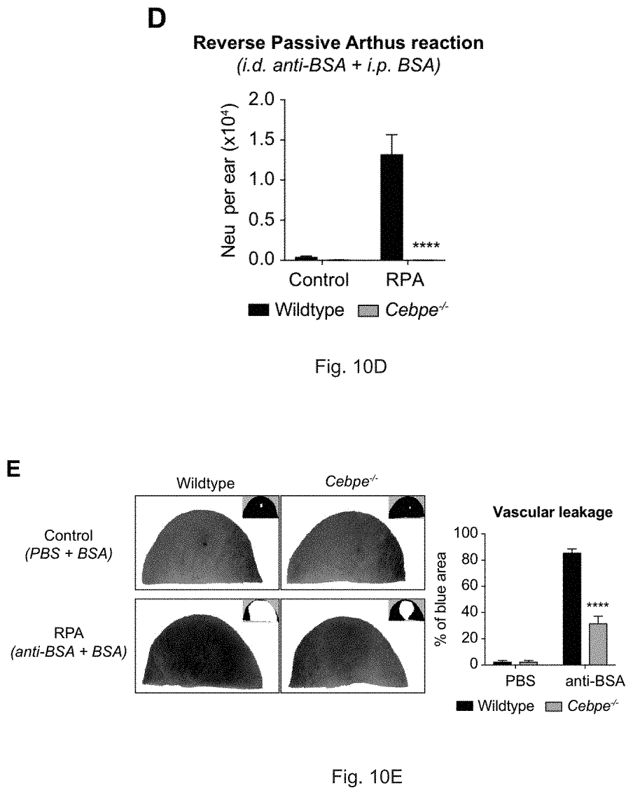

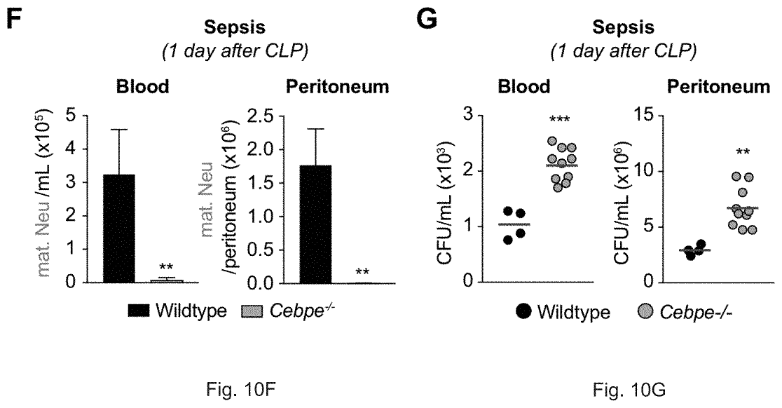

C/EBP_{ε} is a crucial TF for the production of secondary granules in mice and human (Gombart et al., 2003; Yamanaka et al., 1997). However, the precise involvement of C/EBP_{ε} in neutrophil development remains unclear. Since a strong upregulation of Cebpe expression was detected in preNeu population (FIG. 9A), the inventors hypothesized that C/EBP_{ε} could be involved in the transition from GMP to preNeu. To examine this, GMP, preNeu, immature and mature Neu numbers in the BM were compared between Cebpe^{-/-} and WT animals (FIG. 10A). It was observed that preNeu and downstream populations were severely reduced while GMP accumulated in Cebpe^{-/-} mice, supporting that Cebpe is important for the transition from GMP to preNeu (FIG. 10A). In contrast, an increase in tpMo and Ly6C^{hi} monocytes numbers in Cebpe^{-/-} mice (FIG. 10B) was found, which could indicate an aberrant differentiation of GMP to the monocyte fate. To confirm the role of C/EBP_{ε} in neutrophil development in a competitive setting, BM chimeras with an equal mixture of CD45.1⁺ WT and CD45.2⁺ Cebpe^{-/-} BM cells (FIG. 10C) were generated. In line with earlier results (FIG. 10A), preNeu and downstream neutrophil populations were not derived from Cebpe^{-/-} cells but WT cells. Since the development of preNeu is highly impacted in C/EBP_{ε}-deficient animals, it was next investigated how a lack of preNeu during inflammatory responses would result in functional consequences. A neutrophil-dependent model of immune complex-mediated inflammation [reverse passive Arthus (RPA) reaction] (Li et al., 2016) was utilized. Here, reduced neutrophil infiltration (FIG. 10D) and vascular leakage (FIG. 10E) at the site of the reaction were observed. A mid-grade cecal ligation and puncture (CLP) sepsis model also revealed that C/EBPE-deficient mice had poorer bacterial clearance in the blood and peritoneal cavity compared to wildtype control mice (FIGS. 10F and 10G).

Together, these data indicate that the absence of preNeu in Cebpe^{-/-} mice results in impaired development of downstream neutrophil populations.

PreNeu Expand in the Bone Marrow and the Spleen During Inflammation.

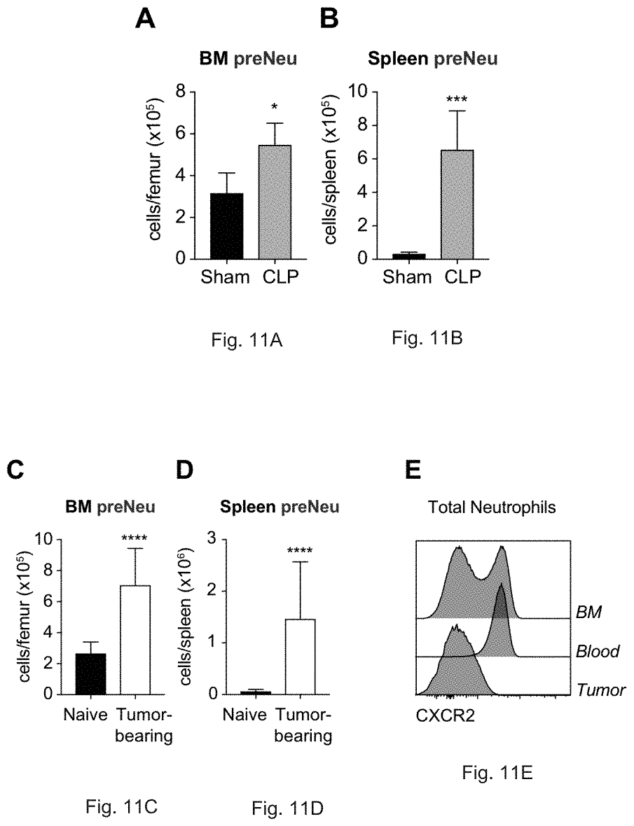

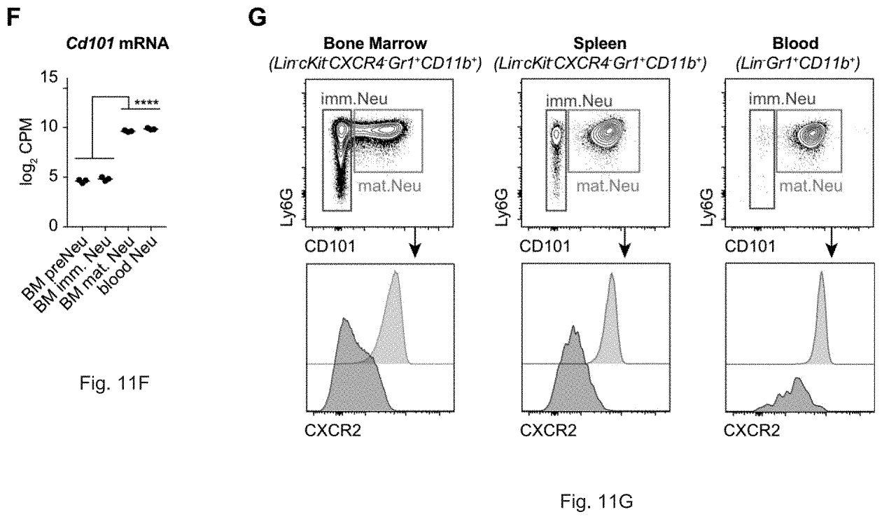

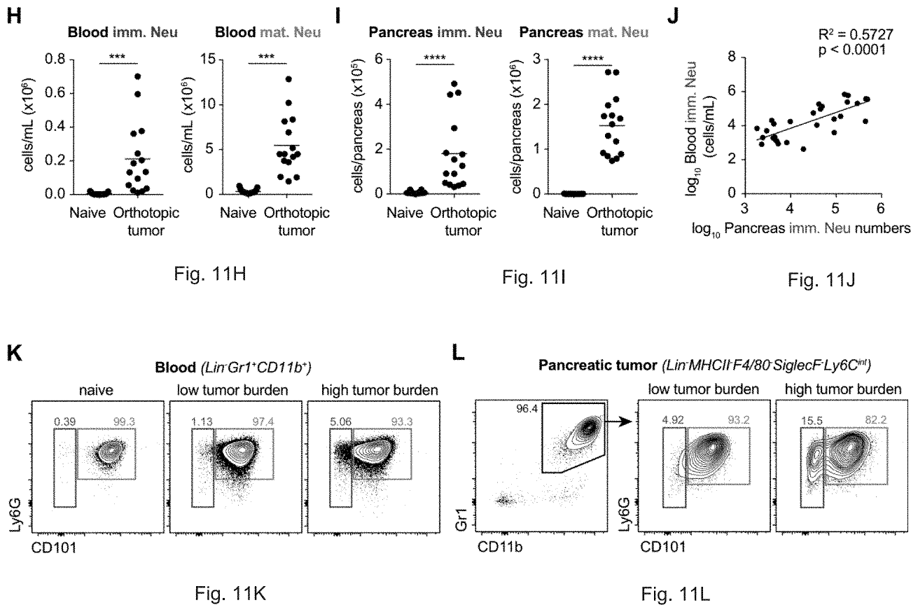

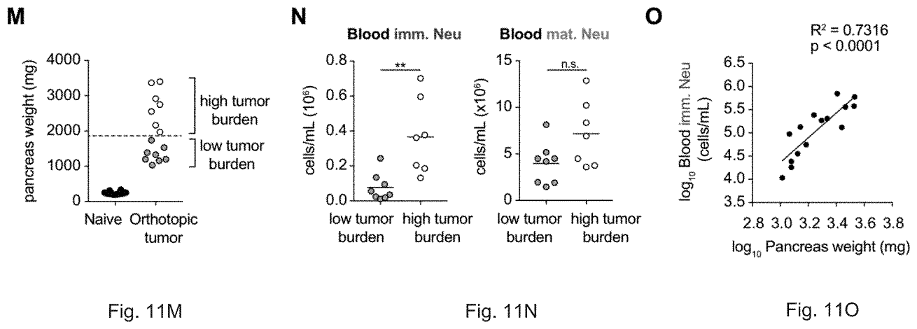

The data (FIG. 10) indicated that the absence of preNeu results in a lack of neutrophil-mediated responses. Since preNeu acted as a proliferative precursor in the steady state, the inventors next sought to understand how preNeu were affected during diseases that require increased myelopoiesis, such as sepsis and cancer. Specifically, an increase in preNeu numbers in the BM and spleen was found upon sepsis (FIGS. 11A-B and 16A). Additionally, a similar effect in an orthotopic tumor model of pancreatic carcinoma (FIGS. 11C-D and 16B) was observed. Of note, while a 2- to 4-fold increase in BM preNeu numbers was detected in both of these models, a significant and drastic >10-fold increase in spleen preNeu numbers (FIGS. 11B and D) was observed. Taken together, the results highlight the expansion of BM and spleen preNeu during inflammatory conditions, indicative of increased intra- and extramedullary granulopoiesis.

CD101^{neg} Immature Neutrophils are Associated with Tumor Progression.

Neutrophils are being increasingly recognized as important players in tumorigenesis. However, conflicting evidences indicate that neutrophils can carry both pro- and anti-tumoral properties (Coffelt et al., 2016; Nicolas-Avila et al., 2017). It is speculated that these opposing observations might be explained by differing maturation status of neutrophils in tumors as recently suggested by others (Coffelt et al., 2016).

To address the hypothesis, it was next investigated whether immature and mature Neu may be identified in orthotopic pancreatic tumors using CXCR2 expression since this marker is differentially expressed between these two populations in the BM (FIG. 7A). However, unlike in the BM and blood, a strong downregulation of CXCR2 in the tumor was found (FIG. 11E).

To overcome the challenge of segregating immature and mature Neu in the tumor, differentially expressed genes (DEGs) were screened for to identify markers that could clearly distinguish these cells in the tumor. Among these DEGs, Cd101, a surface marker that was significantly upregulated in BM mature and blood Neu (FIG. 11F) was identified. To validate, Gr1⁺CD11b⁺ neutrophils in BM, blood and spleen were identified through gating strategies as previously shown (FIGS. 14A and 14B). Importantly, segregating neutrophils with CD101 allowed us to distinguish two populations of neutrophils that matched the expression pattern of CXCR2, such that immature Neu could be defined as Ly6G^{lo/+}CXCR2⁻CD101⁻ while mature Neu could be defined as Ly6G⁺CXCR2⁺CD101⁺ (FIG. 11G). Notably, CD101⁻ immature Neu were nearly absent from the circulation at baseline conditions (FIG. 11G).

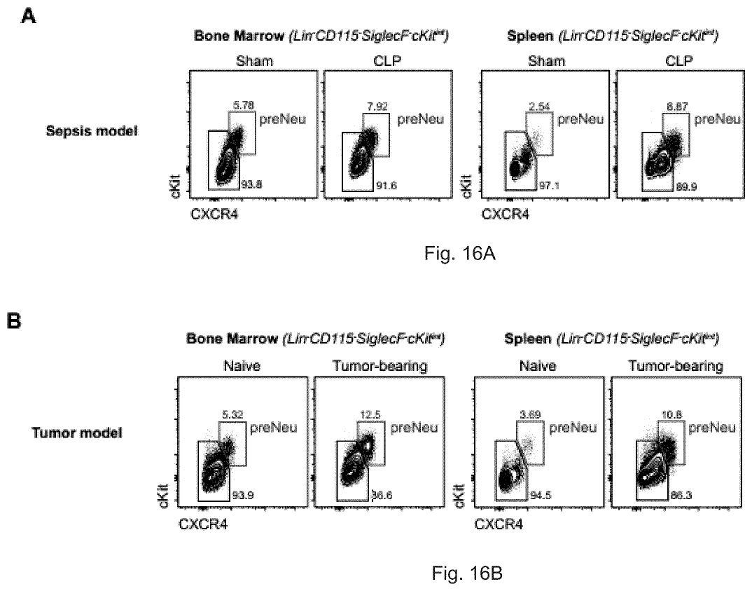

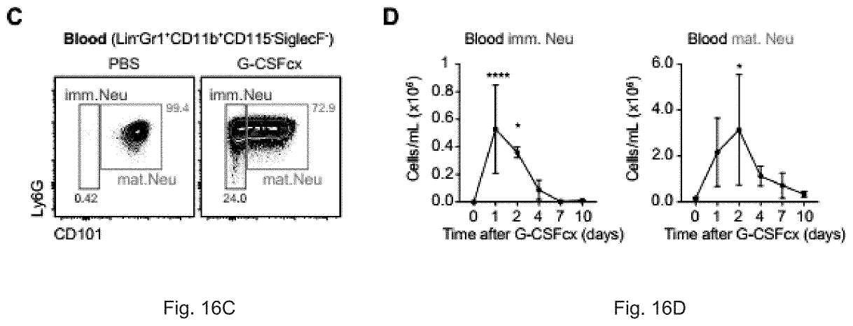

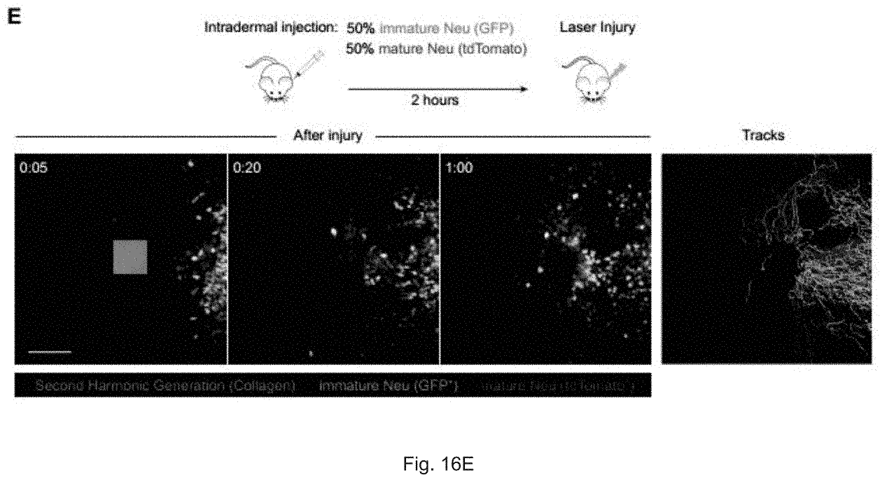

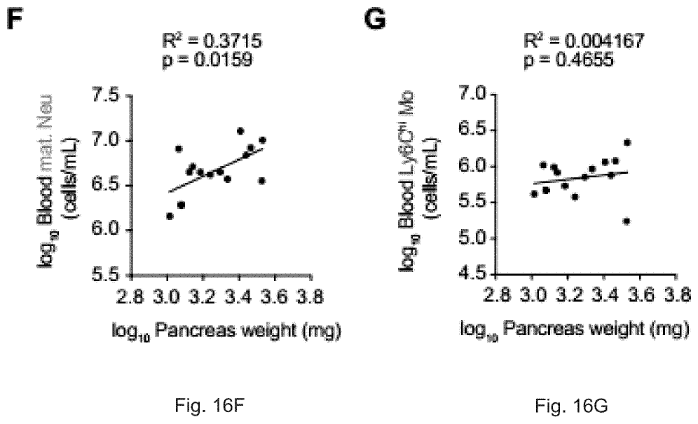

Using CD101 to distinguish immature Neu, it was first determined if this approach would allow for detection of them in the circulation. G-CSF, a strong neutrophil mobilizer that is similarly upregulated during cancer (Kowanetz et al., 2010), was administered into mice. Using this stimulus, immature Neu were detected in the blood and their numbers were maintained in the circulation for up to four days after treatment (FIG. 16C-D). Since immature Neu are able to enter the circulation, the inventors next addressed whether these cells had the capacity to migrate into tissues. Both immature and mature Neu could swarm equally towards the injury core in a sterile laser injury model (FIG. 16E). This is in sharp contrast to preNeu that displayed poor interstitial motility (FIG. 9H) and suggests that immature Neu already possess functional migratory machinery.

Having established that CD101 segregates immature Neu from mature Neu during G-CSF stimulation, this strategy was next validated in the tumor setting. Compared to naïve mice, mice bearing pancreatic tumors showed increased numbers of immature Neu in the blood and pancreas (FIG. 11H-I). Furthermore, a positive correlation between the number of immature Neu in the blood and the pancreas suggested that these cells were actively recruited to the tumor site from the circulation (FIG. 11J). To test whether infiltration of immature Neu contributed to tumoral progression, tumor-bearing mice were separated into two groups according to their tumor weight, and it was found that mice with a higher tumor burden showed higher infiltration of immature Neu into the pancreas (FIG. 11K-M). Additionally, mice with a higher tumor burden had significantly more immature Neu, but no significant differences in mature Neu in the circulation (FIG. 11N). Notably, the number of immature Neu in the blood was highly correlated with the weight of the pancreas (FIG. 11O). In contrast, circulating mature Neu and Ly6C^{hi} monocytes poorly correlated with the pancreas weight (FIG. 16F-G), which suggests that the presence of immature Neu in the blood may serve as a biomarker of disease progression.

In summary, the inventors have identified a strategy to distinguish immature from mature Neu in cancer and reveal that circulating immature Neu numbers are associated with increased tumor burden.

### Identification of Human Neutrophil Subsets Using Proliferative Activity

Cellular proliferation is central to hematopoiesis. The classical model suggests a hierarchical order, which begins with the cellular amplification of hematopoietic stem cells (HSCs) that ultimately leads to the generation of all blood cell lineages. Upon the differentiation of small numbers of slow proliferating HSCs to hematopoietic progenitor/precursor cells (HPCs), HPCs begin to commit towards their respective cell lineages by reducing their capacity for self-renewal and instead proliferate extensively to meet the demand of mature lineage specific cells. Among HPCs, the granulocyte-monocyte progenitor (GMP) gives rise to monocytes, dendritic cells and granulocyte populations such as neutrophils, eosinophils and basophils. More recently, committed progenitors downstream of the GMP, including the common monocyte progenitor (cMoP) that can only form monocytes have been formally identified. However, the developmental trajectory from GMP to functionally mature neutrophils remains poorly defined.

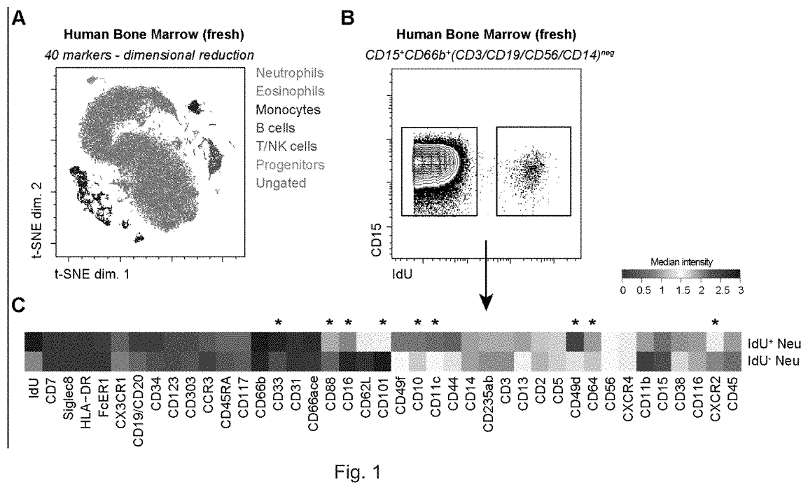

To solve this issue, the inventors employed mass cytometry and measured the expression of 40 different markers to deeply phenotype human bone marrow leukocyte populations. The inventors next utilized the t-distributed Stochastic Neighbor Embedding (t-SNE) algorithm to visualize similarities between cells on a 2D map (FIG. 1A). By doing so, all major lineages could be identified, with neutrophils being the most important cell type in terms of frequency (FIG. 1A). The inventors also used 5-ido-2′-deoxyuridine (IdU), which is readily detected by mass cytometry, to detect cells in the S phase of the cell cycle. Since BM progenitors/precursors undergo extensive proliferation, the inventors hypothesized that a putative neutrophil precursor would be highly proliferative, and therefore would be able to incorporate IdU. Thus, the inventors manually identified CD15⁺CD66⁺ total neutrophils, and gated IdU+ proliferative neutrophils and IdU− non-proliferative neutrophils (FIG. 1B). Median expression of surface markers between these two populations were next plotted onto a heat map to identify markers that could distinguish a putative neutrophil precursor from the other neutrophils (FIG. 1C). Using this approach, the inventors found differentially expressed markers between proliferative and non-proliferative neutrophils that include CD10, CD16, CD49d and CD101.

### Phenotypic Characterisation of Human Neutrophil Subsets

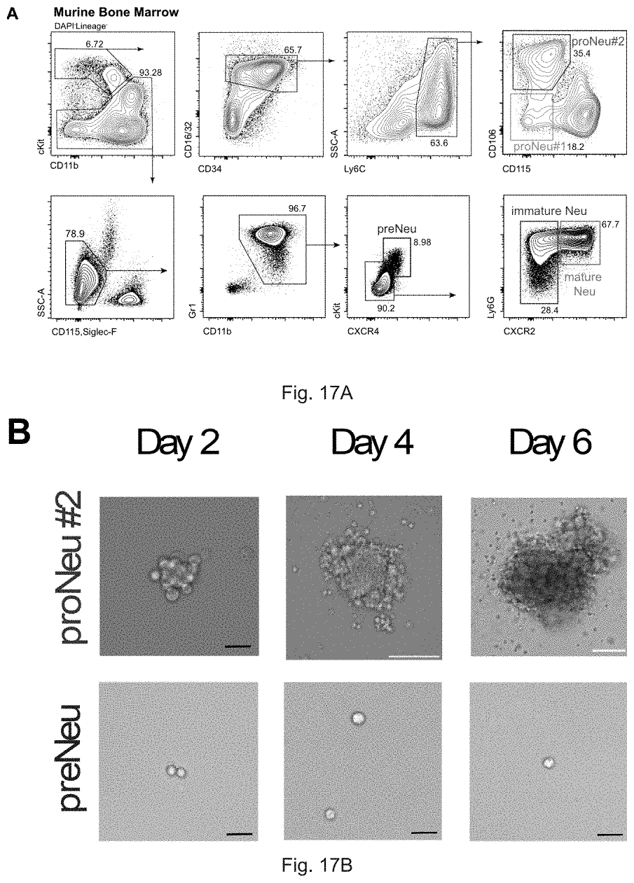

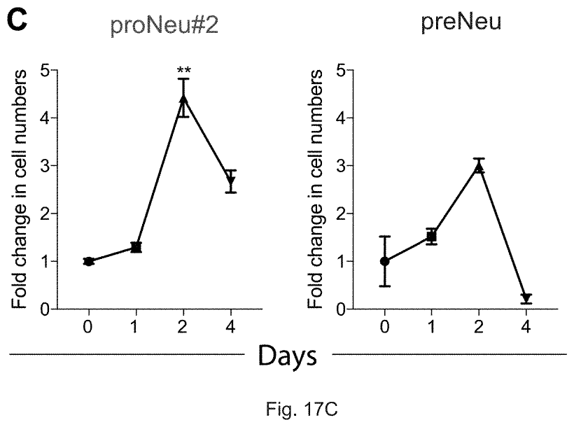

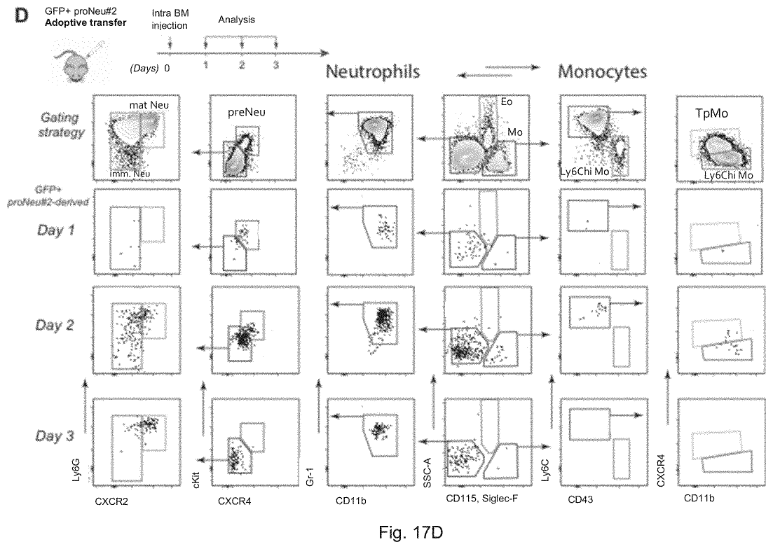

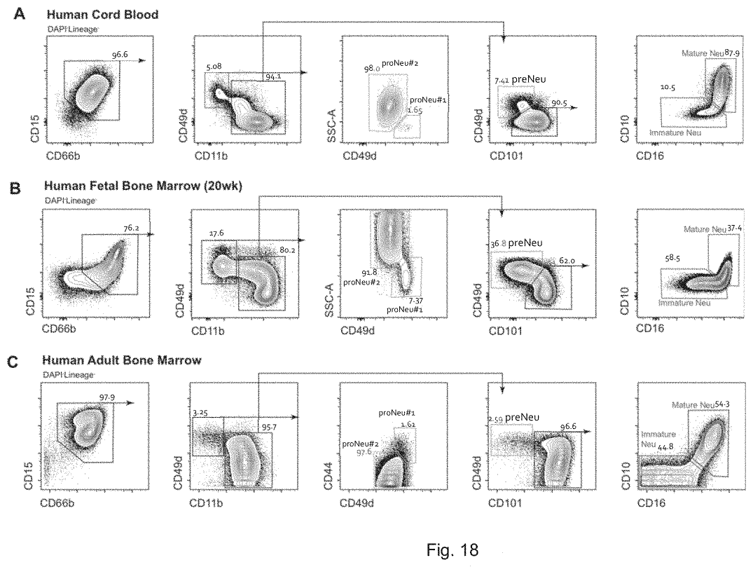

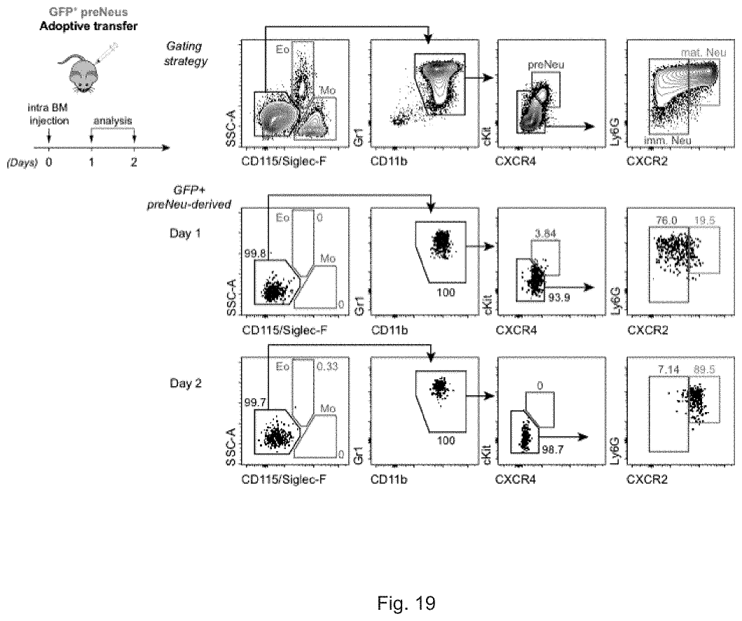

After finding differentially expressed markers between proliferative and non-proliferative neutrophils, the inventors next employed these markers to formally identify a neutrophil precursor population. For this purpose, the inventors manually gated lineage negative cells (CD3/CD19/CD56/CD14), excluded early progenitors (CD34⁺) and eosinophils (Siglec8⁺ or SiglecF⁺) to obtain CD15⁺CD66b⁺ total neutrophils (FIG. 2A). From total neutrophils, the inventors found a population of CD49d⁺CD101⁻ neutrophils that matched the profile of IdU+ proliferative neutrophils and named this population pre-neutrophils (preNeu) (FIG. 2A). The inventors have also found another population of CD49d⁺CD101⁻ neutrophils and named this population pro-neutrophils (proNeu) (FIG. 17, FIG. 18, and FIG. 19).

While it is reported that blood neutrophils show a homogeneous expression of CD10 and CD16, the inventors found that BM neutrophils were heterogeneous for these two markers. The inventors thus defined mature neutrophils that resemble blood neutrophils as CD10⁺CD16⁺, and discovered a population of immature neutrophils negative for both of these markers (FIG. 2A).

After delineating these three BM neutrophil subsets, the inventors screened 39 surface markers to deeply phenotype these populations (FIG. 2B). From these markers, the inventors identified CD10 and CD101 as the most informative and defined preNeu as CD10⁻CD101⁻, immature neutrophils as CD10⁻CD101⁺ and mature neutrophils as CD10⁺CD101⁺ (FIG. 2C).

After characterising the phenotype of human BM neutrophil subsets, the inventors next investigated their tissue distribution. While preNeu and immature neutrophils were found at relatively high frequency within the BM, these subsets were mostly absent from the peripheral circulation (FIG. 3A). This is in line with a tight regulation of neutrophil development, such that only fully mature neutrophils have the ability to egress the BM and enter the bloodstream under resting conditions.

To understand whether these neutrophils subsets were similar across tissues, the inventors used IdU incorporation to probe into the cell cycle status of these cells. Here, the inventors found that preNeu showed similar IdU incorporation in the BM and blood, suggesting that neutrophil subsets display similar proliferation capacity in different tissues (FIG. 3B).

### Application of the Neutrophil Identification Strategy and Isolation

To date, there are no good classification methods for neutrophil development. With this newly established method, subsets of neutrophils at various developmental stages can be identified. The current approach will allow proper characterisation of the presence of different subsets of neutrophils in the circulation or in inflamed tissue/organ (e.g. tumor), as neutrophils are the main leukocytes mobilized/recruited in response to inflammatory responses, thus the frequency of specific subsets of neutrophils can be used as disease prognostic/predictive markers.

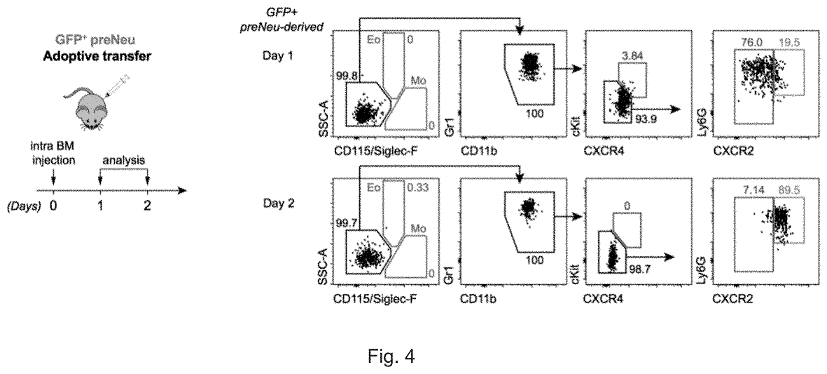

Since preNeu are shown to have proliferative capacity, the inventors have tested the cell lineage commitment of (mouse) preNeu, and the ability of these precursors to repopulate neutrophils in preclinical model (FIG. 4). To this end, the inventors observed that transferred preNeu specifically differentiate into mature CD10+CD101+ neutrophils but not other myeloid cells, indicating that these precursors may be transplanted to immune-compromised patients, such as chemotherapy patients, to temporarily boost their neutrophil counts in the blood for protection against infections. Since preNeu can be transferred and proliferate, preNeu can be a valuable treatment option to replace and/or supplement daily transfusions. Transfer of preNeu rather than mature neutrophils can extend the time between treatments, for example, a three (3) to five (5) days turnover time may be expected for transfer of preNeu.

Pro-Neutrophils

Materials and Method

Processing of Cells for Flow Cytometry and FACS

Bone marrow cells from wild-type mice were obtained by gently crushing bone marrow femora, tibias, pelvis bones, humeri, and spine bones in PBS containing 2% fetal bovine serum (FBS) and 2 mM EDTA. For human samples, cells were obtained from consent-taken donors according to their respective Institutional Review Board (IRBs). Samples were then lysed with 1× red blood cell (RBC) lysis buffer (eBioscience) for 5 min and washed with PBS, spun down at 400 g for 5 min. Samples were then stained with Fc-blocker (human or mouse respectively) for 15 min before adding the appropriate fluorophore-conjugated antibodies for 20 min at 4° C. Cells were then washed before analysing using the BD ARIAII for cell sorting or BD LSRII for analysis purposes.

Adoptive Cell Transfer

Sorted uGFP+ proNeus (1×10⁵ cells) were transferred intra-BM into wild-type recipients as described previously (Chong 2016). Briefly, recipient mice were anesthetized with ketamine (150 mg/kg)/xylazine (10 mg/kg), and had their right leg shaved to expose the kneecap. Sorted proNeus were resuspended in 1× PBS at a concentration of 1×10⁴ cells/μL, and a volume of 10 μL was administered into the tibia through the kneecap using a 29-gauge insulin needle. At 24, 48 and 60 hours after cell transfer, tibias were collected, stained and analyzed by flow cytometry.

In Vitro Proliferation Assay

Sorted cells (3×10⁴ for each cell subset) were plated onto 96-well plates in triplicates and cultured at 37° C., 5% CO₂ in Iscove's Modified Dulbecco's Medium with 25 mM HEPES and L-Glutamine (Chemtron) containing 10% (vol/vol) FBS, 1 mM sodium pyruvate, penicillin (100 U/ml) and streptomycin (100 ug/ml). A combination of 50 ng/ml SCF, 20 ng/ml LIF, 10 ng/ml IL-3, 20 ng/ml IL-6 (all from StemCell Technologies) was added to the cell culture medium. Cells were then analyzed over a period of 4 days.

Colony Formation Assay

Sorted cells (3×10⁴ for each cell subset) were plated onto 60 mm dishes in duplicates and cultured at 37° C., 5% CO₂ in 2% Methylcellulose MethoCult™ Medium with 25 mM HEPES and L-Glutamine (Chemtron) containing 10% (vol/vol) FBS, 1 mM sodium pyruvate, penicillin (100 U/ml) and streptomycin (100 ug/ml). A combination of 50 ng/ml SCF, 20 ng/ml LIF, 10 ng/ml IL-3, 20 ng/ml IL-6 (all from StemCell Technologies) was added to the cell culture medium.

Transcriptional Regulation of Neutrophil Precursors

Indicated progenitor subsets were single-cell sorted accordingly into 96-well plates containing 10 mM of dNTP and 1% BSA. Single-cell lysis was performed using 1 μl of RNase inhibitor to 19 μl of a 0.2% (vol/vol) Triton X-100 solution. Cells were incubated at 72° C. for 3 min and then spun down. Reverse transcription and PCR steps were performed according to the manufacturer's protocol (illumina). DNA was then sequenced with a HiSeq 2500. RNA-Seq data in the form of FASTQ files were subsequently mapped to the mouse genome build mm10 using the STAR alignment software. The mapped reads were then counted using featureCounts (part of Subread package) based on the GENCODE M7 annotations. Data was then analysed using Seurat.

Results

Phenotypic Information

Murine pro-neutrophils (proNeus) are characterised by cKit^{hi}Ly6C⁺CD106⁺CD115⁻CD205⁻CD11b^{lo}Gr1^{lo}. Murine pre-neutrophils (preNeus) are instead characterised by cKit^{lo}Ly6C⁺SiglecF⁻CD115⁻CD205⁺CD11b^{hi}Gr1^{hi}CXCR4^{hi}.

Human pro-neutrophils (proNeus) are defined by CD34⁻ CD66b⁺CD15⁺CD71⁺CD49d⁺CD101⁻CD11b⁻. Human pre-neutrophils (preNeus) are instead characterised by CD66b⁺CD15⁺CD71⁺CD49d⁺CD101⁻CD11b⁺.

These differences can be clearly seen in FIG. 17A and FIG. 18.

Comparisons with Pre-Neutrophils

Besides their phenotypic differences, pro-neutrophils (proNeu) are higher in proliferation potential compared to pre-neutrophils (preNeus). This is seen clearly by FIG. 17B and FIG. 17C. Ongoing studies are being done to show this difference in the human neutrophil precursors.

Also, in terms of neutrophil ontogeny, pro-neutrophils (proNeus) are earlier in differentiation compared to pre-neutrophils (preNeus). This is supported in the in vivo data in FIG. 17D as pro-neutrophils (proNeus) can differentiate into pre-neutrophils (preNeus) after 1 day.

It should be further appreciated by the person skilled in the art that variations and combinations of features described above, not being alternatives or substitutes, may be combined to form yet further embodiments falling within the intended scope of the invention.

Transcriptional Regulation of Neutrophil Precursors

At the single-cell level, pro-neutrophils (proNeus) are transcriptomically distinct from pre-neutrophils (preNeus), as shown in FIG. 20A. Pro-neutrophils (proNeus) express much higher levels of primary granules related genes compared to monocyte precursors (cMoPs) and pre-neutrophils (preNeus). Furthermore, the specification of neutrophil commitment is further appreciated in expression levels of the key known transcription factors required for neutrophil differentiation shown in FIG. 20B. Common neutrophil-related genes described in the literature (Giladi et al., 2018, Yanez et al., 2018, Olsson et al., 2016) was also noted in FIG. 20C. These genes were thought to represent one subset of precursor cells. However, data in the present disclosure shows both exclusive and shared gene signatures between pro-neutrophils (proNeus) and pre-neutrophils (preNeus).

While neutrophil heterogeneity is increasingly appreciated, their developmental path and functional properties from multipotent GMP to mature neutrophils remains elusive (Silvestre-Roig et al., 2016). Here, the inventors have established a methodological framework with the latest analytical approaches to identify and provide an in-depth functional characterisation of neutrophil subsets in their developmental pathway. Specifically, the inventors identified a proliferative neutrophil precursor population, which the inventors termed pro-neutrophils (proNeu) and pre-neutrophils (preNeu), that gives rise to an intermediate population (immature Neu) in the BM before differentiating into mature neutrophils. The sepsis and tumor models employed further revealed distinctive roles for proNeu, preNeu and immature Neu, with the numbers of immature Neu correlating with tumor burden. More importantly, the inventors also resolved an ongoing challenge of distinguishing neutrophil subsets during inflammation by identifying CD101 as a marker that segregates immature Neu from mature Neu in the circulation and tumor site. The study hence fills a long-standing gap in the neutrophil development pathway by providing a framework to better understand the functional characteristics of neutrophil subsets in both steady and inflammatory states.

Neutrophil development has been characterised historically through a density gradient separation technique, followed by their identification with Giemsa stain (Bjerregaard et al., 2003). While this approach delivers useful insights about neutrophil development and maturation, it lacks precision in delineating neutrophil heterogeneity at the single cell resolution and does not allow downstream functional and molecular characterisation. Here, mass cytometry-based analytical approaches were utilized to identify differentially expressed surface markers on proliferating hematopoietic cells. These surface markers were then incorporated into subsequent flow cytometric analysis in the Fucci-(S-G2-M) mouse, which led the inventors to identify three BM neutrophil subsets, namely: preNeu, immature Neu and mature Neu. Of note, this approach was robust in both mice and humans, and could also be applied to other leukocyte populations. On further investigation, the inventors also found a fourth BM neutrophil subset of pro-neutrophils (proNeu).

While the commitment of multipotent precursors to cell-restricted precursors involves upregulating and silencing of lineage-specific and irrelevant genes respectively (Fiedler and Brunner, 2012), how precursors commit towards the neutrophil lineage remains unclear. The transcriptomic analysis of BM neutrophil subsets revealed TF silencing of Irf8, Gata1, Gata2 and activation of Gfi1 and Cebpe in preNeu. Gfi1 is known to be critical for multipotent precursor commitment to the granulocytic lineage (Hock et al., 2003). Thus, a specific upregulation of Gfi1 expression in proNeu and/or preNeu further suggests that these cells are the first precursors committed towards the neutrophil lineage. The inventors confirmed the importance of C/EBPε in the development of preNeu to downstream neutrophil populations, as functionally mature neutrophils were absent in Cebpe^{-/-} mice. The inventors have also validated the developmental hierarchy of neutrophils to corroborate the notion that proliferative proNeu and/or preNeu undergo an intermediate developmental phase of immature Neu before differentiating into functionally mature Neu. In alignment with this discovery, it is believed that mapping of this trajectory in humans would provide further insights into the current established neutrophil development hierarchy.

Myeloid precursor expansion during emergency granulopoiesis is necessary to meet the demand for functionally mature neutrophils (Manz and Boettcher, 2014). Consequently, the sepsis and tumor models demonstrate an expansion of preNeu in both the spleen and BM during inflammation. Two possibilities may account for this phenomenon: an activation of extramedullary granulopoiesis in the spleen; or deployment of BM preNeu to extramedullary sites in response to inflammatory stimuli. The current findings rule out the latter possibility as preNeu were absent in the circulation (data not shown) and are poorly motile. The sessile nature of proliferative preNeu is in line with the “go or grow” hypothesis in cancer biology, which postulates that cytoskeleton machineries are unable to cater to the needs of proliferation and migration simultaneously (Garay et al., 2013). Therefore, the expansion of splenic preNeu is most likely attributed to heightened extramedullary granulopoiesis through increased production of GM-CSF and IL-3 in the spleen microenvironment (Weber et al., 2015).

In contrast to proNeu and/or preNeu, immature Neu are non-proliferative but can enter the bloodstream during inflammatory conditions. Importantly, immature Neu could migrate towards the site of injury as efficiently as mature Neu. These data hence suggest that while proNeu and/or preNeu are proliferative precursors that fine-tune the output of neutrophils; immature Neu may serve as a reservoir that can be deployed to sites of inflammation instead. It is currently unclear what the implications of this “premature” mobilization of immature Neu to the circulation and local sites of inflammation are. Nevertheless, the tumor studies indicate a strong correlation between circulating immature Neu numbers and tumor burden, suggesting that their numbers could be used as a prognostic measurement of tumor burden. These findings corroborate recent observations of neutrophils with immature or aberrant nuclear morphology in tumor-bearing mice (Coffelt et al., 2015) that have been negatively associated with disease outcome (Sagiv et al., 2015; Yang et al., 2011). The study highlights an important role for immature Neu during diseases and unlocks potential research topics on the relationship between immature Neu and granulocytic myeloid-derived suppressor cells (G-MDSC).

In summary, the study provides an advancement in the understanding of neutrophil development by identifying specialized granulocytic populations that ensure supply during homeostasis and early response under stress. More importantly, the current model may also serve as a fundamental platform for the re-examination of granulopoiesis under physiological and disease states, as well as the basis for new therapeutic interventions for neutrophil-related diseases.

### REFERENCES

1. Akashi, K., Traver, D., Miyamoto, T., and Weissman, I. L. (2000). A clonogenic common myeloid progenitor that gives rise to all myeloid lineages. Nature 404, 193-197.
2. Anthony, B. A., and Link, D. C. (2014). Regulation of hematopoietic stem cells by bone marrow stromal cells. Trends Immunol 35, 32-37.
3. Balabanian, K., Brotin, E., Biajoux, V., Bouchet-Delbos, L., Lainey, E., Fenneteau, O., Bonnet, D., Fiette, L., Emilie, D., and Bachelerie, F. (2012). Proper desensitization of CXCR4 is required for lymphocyte development and peripheral compartmentalization in mice. Blood 119, 5722-5730.
4. Becher, B., Schlitzer, A., Chen, J., Mair, F., Sumatoh, H. R., Teng, K. W., Low, D., Ruedl, C., Riccardi-Castagnoli, P., Poidinger, M., et al. (2014). High-dimensional analysis of the murine myeloid cell system. Nat Immunol 15, 1181-1189.
5. Bjerregaard, M. D., Jurlander, J., Klausen, P., Borregaard, N., and Cowland, J. B. (2003). The in vivo profile of transcription factors during neutrophil differentiation in human bone marrow. Blood 101, 4322-4332.
6. Borregaard, N. (2010). Neutrophils, from marrow to microbes. Immunity 33, 657-670.
7. Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128.
8. Chen, H., Lau, M. C., Wong, M. T., Newell, E. W., Poidinger, M., and Chen, J. (2016). Cytofkit: A Bioconductor Package for an Integrated Mass Cytometry Data Analysis Pipeline. PLoS Comput Biol 12, e1005112.
9. Chong, S. Z., Evrard, M., Devi, S., Chen, J., Lim, J. Y., See, P., Zhang, Y., Adrover, J. M., Lee, B., Tan, L., et al. (2016). CXCR4 identifies transitional bone marrow premonocytes that replenish the mature monocyte pool for peripheral responses. J Exp Med 213, 2293-2314.
10. Chua, R. Y., and Wong, S. H. (2013). SNX3 recruits to phagosomes and negatively regulates phagocytosis in dendritic cells. Immunology 139, 30-47.
11. Coffelt, S. B., Kersten, K., Doornebal, C. W., Weiden, J., Vrijland, K., Hau, C. S., Verstegen, N. J., Ciampricotti, M., Hawinkels, L. J., Jonkers, J., and de Visser, K. E. (2015). IL-17-producing gammadelta T cells and neutrophils conspire to promote breast cancer metastasis. Nature 522, 345-348.
12. Coffelt, S. B., Wellenstein, M. D., and de Visser, K. E. (2016). Neutrophils in cancer: neutral no more. Nat Rev Cancer 16, 431-446.
13. Devi, S., Wang, Y., Chew, W. K., Lima, R., N, A. G., Mattar, C. N., Chong, S. Z., Schlitzer, A., Bakocevic, N., Chew, S., et al. (2013). Neutrophil mobilization via plerixafor-mediated CXCR4 inhibition arises from lung demargination and blockade of neutrophil homing to the bone marrow. J Exp Med 210, 2321-2336.
14. Faust, N., Varas, F., Kelly, L. M., Heck, S., and Graf, T. (2000). Insertion of enhanced green fluorescent protein into the lysozyme gene creates mice with green fluorescent granulocytes and macrophages. Blood 96, 719-726.
15. Fiedler, K., and Brunner, C. (2012). The role of transcription factors in the guidance of granulopoiesis. Am J Blood Res 2, 57-65.
16. Ford, A. M., Bennett, C. A., Healy, L. E., Towatari, M., Greaves, M. F., and Enver, T. (1996). Regulation of the myeloperoxidase enhancer binding proteins Pu1, C-EBP alpha, -beta, and -delta during granulocyte-lineage specification. Proc Natl Acad Sci USA 93, 10838-10843.
17. Frenette, P. S., Pinho, S., Lucas, D., and Scheiermann, C. (2013). Mesenchymal stem cell: keystone of the hematopoietic stem cell niche and a stepping-stone for regenerative medicine. Annu Rev Immunol 31, 285-316.
18. Garay, T., Juhasz, E., Molnar, E., Eisenbauer, M., Czirok, A., Dekan, B., Laszlo, V., Hoda, M. A., Dome, B., Timar, J., et al. (2013). Cell migration or cytokinesis and proliferation?—revisiting the “go or grow” hypothesis in cancer cells in vitro. Exp Cell Res 319, 3094-3103.
19. Ginhoux, F., and Jung, S. (2014). Monocytes and macrophages: developmental pathways and tissue homeostasis. Nat Rev Immunol 14, 392-404.
20. Gombart, A. F., Kwok, S. H., Anderson, K. L., Yamaguchi, Y., Torbett, B. E., and Koeffler, H. P. (2003). Regulation of neutrophil and eosinophil secondary granule gene expression by transcription factors C/EBP epsilon and PU.1. Blood 101, 3265-3273.
21. Hahsler, M., Hornik, K., and Buchta, C. (2008). Getting things in order: an introduction to the R package seriation. Journal of Statistical Software 25, 1-34.
22. Hettinger, J., Richards, D. M., Hansson, J., Barra, M. M., Joschko, A. C., Krijgsveld, J., and Feuerer, M. (2013). Origin of monocytes and macrophages in a committed progenitor. Nat Immunol 14, 821-830.
23. Hock, H., Hamblen, M. J., Rooke, H. M., Traver, D., Bronson, R. T., Cameron, S., and Orkin, S. H. (2003). Intrinsic requirement for zinc finger transcription factor Gfi-1 in neutrophil differentiation. Immunity 18, 109-120.
24. Kim, H. K., De La Luz Sierra, M., Williams, C. K., Gulino, A. V., and Tosato, G. (2006). G-CSF down-regulation of CXCR4 expression identified as a mechanism for mobilization of myeloid cells. Blood 108, 812-820.
25. Kohler, A., De Filippo, K., Hasenberg, M., van den Brandt, C., Nye, E., Hosking, M. P., Lane, T. E., Mann, L., Ransohoff, R. M., Hauser, A. E., et al. (2011). G-CSF-mediated thrombopoietin release triggers neutrophil motility and mobilization from bone marrow via induction of Cxcr2 ligands. Blood 117, 4349-4357.
26. Kolaczkowska, E., and Kubes, P. (2013). Neutrophil recruitment and function in health and inflammation. Nat Rev Immunol 13, 159-175.
27. Kowanetz, M., Wu, X., Lee, J., Tan, M., Hagenbeek, T., Qu, X., Yu, L., Ross, J., Korsisaari, N., Cao, T., et al. (2010). Granulocyte-colony stimulating factor promotes lung metastasis through mobilization of Ly6G+Ly6C+ granulocytes. Proceedings of the National Academy of Sciences 107, 21248-21255.
28. Kyme, P., Thoennissen, N. H., Tseng, C. W., Thoennissen, G. B., Wolf, A. J., Shimada, K., Krug, U. O., Lee, K., Muller-Tidow, C., Berdel, W. E., et al. (2012). C/EBPepsilon mediates nicotinamide-enhanced clearance of Staphylococcus aureus in mice. J Clin Invest 122, 3316-3329.
29. Lahoz-Beneytez, J., Elemans, M., Zhang, Y., Ahmed, R., Salam, A., Block, M., Niederalt, C., Asquith, B., and Macallan, D. (2016). Human neutrophil kinetics: modeling of stable isotope labeling data supports short blood neutrophil half-lives. Blood 127, 3431-3438.
30. Li, J. L., Goh, C. C., Keeble, J. L., Qin, J. S., Roediger, B., Jain, R., Wang, Y., Chew, W. K., Weninger, W., and Ng, L. G. (2012). Intravital multiphoton imaging of immune responses in the mouse ear skin. Nat Protoc 7, 221-234.
31. Li, J. L., Lim, C. H., Tay, F. W., Goh, C. C., Devi, S., Malleret, B., Lee, B., Bakocevic, N., Chong, S. Z., Evrard, M., et al. (2016). Neutrophils Self-Regulate Immune Complex-Mediated Cutaneous Inflammation through CXCL2. J Invest Dermatol 136, 416-424.
32. Manz, M. G., and Boettcher, S. (2014). Emergency granulopoiesis. Nat Rev Immunol 14, 302-314.
33. Naik, S. H., Sathe, P., Park, H. Y., Metcalf, D., Proietto, A. I., Dakic, A., Carotta, S., O'Keeffe, M., Bahlo, M., Papenfuss, A., et al. (2007). Development of plasmacytoid and conventional dendritic cell subtypes from single precursor cells derived in vitro and in vivo. Nat Immunol 8, 1217-1226.
34. Nicolas-Avila, J. A., Adrover, J. M., and Hidalgo, A. (2017). Neutrophils in Homeostasis, Immunity, and Cancer. Immunity 46, 15-28.
35. Orkin, S. H., and Zon, L. I. (2008). Hematopoiesis: an evolving paradigm for stem cell biology. Cell 132, 631-644.
36. Passegue, E., Wagner, E. F., and Weissman, I. L. (2004). JunB deficiency leads to a myeloproliferative disorder arising from hematopoietic stem cells. Cell 119, 431-443.
37. Picelli, S., Faridani, O. R., Bjorklund, A. K., Winberg, G., Sagasser, S., and Sandberg, R. (2014). Full-length RNA-seq from single cells using Smart-seq2. Nat Protoc 9, 171-181.
38. Reber, L. L., Gillis, C. M., Starkl, P., Jonsson, F., Sibilano, R., Marichal, T., Gaudenzio, N., Berard, M., Rogalla, S., Contag, C. H., et al. (2017). Neutrophil myeloperoxidase diminishes the toxic effects and mortality induced by lipopolysaccharide. J Exp Med 214, 1249-1258.
39. Rittirsch, D., Huber-Lang, M. S., Flierl, M. A., and Ward, P. A. (2009). Immunodesign of experimental sepsis by cecal ligation and puncture. Nat Protoc 4, 31-36.
40. Rubinstein, M. P., Salem, M. L., Doedens, A. L., Moore, C. J., Chiuzan, C., Rivell, G. L., Cole, D. J., and Goldrath, A. W. (2013). G-CSF/anti-G-CSF antibody complexes drive the potent recovery and expansion of CD11b+Gr-1+ myeloid cells without compromising CD8+ T cell immune responses. J Hematol Oncol 6, 75.
41. Sagiv, J. Y., Michaeli, J., Assi, S., Mishalian, I., Kisos, H., Levy, L., Damti, P., Lumbroso, D., Polyansky, L., Sionov, R. V., et al. (2015). Phenotypic diversity and plasticity in circulating neutrophil subpopulations in cancer. Cell Rep 10, 562-573.
42. Sakaue-Sawano, A., Kurokawa, H., Morimura, T., Hanyu, A., Hama, H., Osawa, H., Kashiwagi, S., Fukami, K., Miyata, T., Miyoshi, H., et al. (2008). Visualizing spatiotemporal dynamics of multicellular cell-cycle progression. Cell 132, 487-498.
43. Silvestre-Roig, C., Hidalgo, A., and Soehnlein, O. (2016). Neutrophil heterogeneity: implications for homeostasis and pathogenesis. Blood 127, 2173-2181.
44. Summers, C., Rankin, S. M., Condliffe, A. M., Singh, N., Peters, A. M., and Chilvers, E. R. (2010). Neutrophil kinetics in health and disease. Trends Immunol 31, 318-324.
45. Tak, T., Tesselaar, K., Pillay, J., Borghans, J. A., and Koenderman, L. (2013). What's your age again? Determination of human neutrophil half-lives revisited. J Leukoc Biol 94, 595-601.
46. Tomura, M., Sakaue-Sawano, A., Mori, Y., Takase-Utsugi, M., Hata, A., Ohtawa, K., Kanagawa, O., and Miyawaki, A. (2013). Contrasting quiescent G0 phase with mitotic cell cycling in the mouse immune system. PLoS One 8, e73801.
47. van der Maaten, L., and Hinton, G. (2008). Visualizing data using t-SNE. The Journal of Machine Learning Research 9, 85.
48. Weber, G. F., Chousterman, B. G., He, S., Fenn, A. M., Nairz, M., Anzai, A., Brenner, T., Uhle, F., Iwamoto, Y., Robbins, C. S., et al. (2015). Interleukin-3 amplifies acute inflammation and is a potential therapeutic target in sepsis. Science 347, 1260-1265.
49. Yamanaka, R., Barlow, C., Lekstrom-Himes, J., Castilla, L. H., Liu, P. P., Eckhaus, M., Decker, T., Wynshaw-Boris, A., and Xanthopoulos, K. G. (1997). Impaired granulopoiesis, myelodysplasia, and early lethality in CCAAT/enhancer binding protein epsilon-deficient mice. Proc Natl Acad Sci USA 94, 13187-13192.
50. Yanez, A., Ng, M. Y., Hassanzadeh-Kiabi, N., and Goodridge, H. S. (2015). IRF8 acts in lineage-committed rather than oligopotent progenitors to control neutrophil vs monocyte production. Blood 125, 1452-1459.
51. Yang, X. D., Ai, W., Asfaha, S., Bhagat, G., Friedman, R. A., Jin, G., Park, H., Shykind, B., Diacovo, T. G., Falus, A., and Wang, T. C. (2011). Histamine deficiency promotes inflammation-associated carcinogenesis through reduced myeloid maturation and accumulation of CD11b+Ly6G+ immature myeloid cells. Nat Med 17, 87-95.
52. Yo, M., Sakaue-Sawano, A., Noda, S., Miyawaki, A., and Miyoshi, H. (2015). Fucci-guided purification of hematopoietic stem cells with high repopulating activity. Biochem Biophys Res Commun 457, 7-11.
53. Yona, S., Kim, K. W., Wolf, Y., Mildner, A., Varol, D., Breker, M., Strauss-Ayali, D., Viukov, S., Guilliams, M., Misharin, A., et al. (2013). Fate mapping reveals origins and dynamics of monocytes and tissue macrophages under homeostasis. Immunity 38, 79-91.
54. Zambirinis, C. P., Levie, E., Nguy, S., Avanzi, A., Barilla, R., Xu, Y., Seifert, L., Daley, D., Greco, S. H., Deutsch, M., et al. (2015). TLR9 ligation in pancreatic stellate cells promotes tumorigenesis. J Exp Med 212, 2077-2094.

### Application

Using mass cytometry (CyTOF) and cell cycle-based analysis, the inventors of the present disclosure are the first to identify four neutrophil subsets within the bone marrow (BM): a proliferative neutrophil precursor including pro-neutrophils (i.e. proNeu) and pre-neutrophils (i.e. preNeu), an immature neutrophil, and a mature neutrophil. Unlike mature neutrophils, pro-neutrophils, pre-neutrophils and immature neutrophils are largely absent from the blood circulation. In addition, screening of surface markers revealed that these neutrophil subsets could be separated by the expression of CD101. In some examples, the neutrophil subsets could be separated by the expression of one or more (or two markers), such as CD101 and/or CD10. The inventors believe that this identification strategy could be of use in cases of inflammation whereby pro-neutrophils (proNeu) and pre-neutrophils (preNeu) subset are expanded in the bone marrow and immature neutrophils are mobilized into the peripheral circulation, which could be used as therapeutic targets. Surprisingly, the use of CD101 to separate two populations of neutrophils has never been described before. Further, the possibility of combining surface markers CD10 and CD101 for the identification/characterisation of four neutrophil populations have also never been described before. The proliferative pro-neutrophils (proNeu) and pre-neutrophils (preNeu) populations are lineage committed and can have potential applications in transfusion therapy.

Current state of art treatment for neutropenic patients (preceding chemotherapy) may include granulocyte transfusions and G-CSF injections. These granulocytes are short-lived and are required in large quantities to confer any protective function. As such, granulocytes transfusions typically must be performed frequently. Using pre-neutrophils, instead, may allow for a more effective way of supplying neutrophils to recipients. Even further, using pro-neutrophils may provide a greater source of neutrophil supply where needed. For example, proliferative neutrophils may be obtained/supplied from a donor who is HLA-matched with the recipient. As would be understood by the person skilled in the art, HLA-matching allows for better engraftment and acceptance of the transplanted cells (i.e. the proliferative neutrophils).

Features of the present disclosure include:
Total neutrophils can be separated into 4 different populations based on cell-cycle activity and cell surface markers identified by mass cytometry.
Proliferative population comprising pro-neutrophils (proNeu) and pre-neutrophils (preNeu) and non-proliferative population comprising immature neutrophils are mainly localized in the bone marrow in healthy patients, unlike mature neutrophils.
These neutrophil subsets can be delineated using one or more surface markers, such as: CD101 or CD10.
Perturbation in the amount of pro-neutrophils (proNeu), pre-neutrophils (preNeu) and immature neutrophils in the blood circulation could be used as a biomarker of inflammation.
Pro-neutrophils (proNeu) may provide a greater source of neutrophil supply in certain cases where needed.

1-54. (canceled)

55. A method of characterising and/or separating neutrophils, the method comprising:
characterising and/or separating the neutrophils into a first population comprising proliferative neutrophils and a second population comprising mature neutrophils, according to the expression of CD101 on the neutrophils.

56. The method according to claim 55, wherein the first population expresses CD101⁻ and the second population expresses CD101⁺.

57. The method according to claim 55, wherein when the neutrophils are human neutrophils, the method further comprises characterising and/or separating the neutrophils according to the expression of CD10 on the neutrophils, wherein the first population comprising proliferative neutrophils are CD10⁻CD101⁻ and the second population comprising mature neutrophils are CD10⁺CD101⁺, optionally the second neutrophils population further comprises immature neutrophils that are CD10⁻CD101⁺, optionally the method further comprises characterising and/or separating the neutrophils according to the expression of one or more biomarkers selected from the group consisting of CD49d, CD16, CXCR2, CD34, CD66, CD15, CD71, and CD11b.

58. The method according to claim 55, wherein the first population expresses CD101⁻ and the second population expresses CD101⁺, wherein when the neutrophils are human neutrophils, the method further comprises characterising and/or separating the neutrophils according to the expression of CD10 on the neutrophils, wherein the first population comprising proliferative neutrophils are CD10⁻CD101⁻ and the second population comprising mature neutrophils are CD10⁺CD101⁺, optionally the second neutrophils population further comprises immature neutrophils that are CD10⁻CD101⁺,
optionally wherein the method further comprises characterising and/or separating the neutrophils according to the expression of one or more biomarkers selected from the group consisting of CD49d, CD16, CXCR2, CD34, CD66, CD15, CD71, and CD11b.

59. The method according to claim 55, wherein the proliferative neutrophils comprise pro-neutrophils and pre-neutrophils,
optionally wherein the pro-neutrophils are CD101⁻CD10⁻CD16⁻CD34⁻ CD66b⁺CD15⁺CD71⁺CD49d⁺CD11b⁻CXCR2⁻, the pre-neutrophils are CD101⁻CD10⁻CD16⁻ CD34⁻CD66b⁺CD15⁺CD71⁺CD49d⁺CD11b⁺CXCR2⁻, the immature neutrophils are CD101⁺CD10⁻CD16⁻CD34⁻CD66b⁺CD15⁺CD71⁻CD49d^{lo}CD11b⁺CXCR2⁻, and the mature neutrophils are CD101⁺CD10⁺CD16⁺CD34⁻CD66b⁺CD15⁺CD71⁻CD49d^{lo}CD11b⁺CXCR2⁺.

60. The method according to claim 55, wherein when the neutrophils are murine neutrophils, the method further comprises characterising and/or separating the neutrophils according to the expression of cKit on the neutrophils, wherein the first population comprising proliferative neutrophils is one of cKit^{hi}CD101⁻, cKit^{int}CD101⁻, or cKit^{lo}CD101⁻ and the second population comprising mature neutrophils are cKit-CD101⁺,
optionally the first neutrophils population further comprises immature neutrophils that are cKit^{lo}CD101⁺.

61. The method according to claim 55, wherein the method further comprising characterising and/or separating the neutrophils according to the expression of one or more biomarkers selected from the group consisting of CD101, cKit, Ly6C, CD106, SiglecF, CD115, CD205, CD11b, Gr1, and CXCR4.

62. The method according to claim 55, wherein the proliferative neutrophils comprise pro-neutrophils and pre-neutrophils, optionally wherein pro-neutrophils are CD101⁻ cKit^{Hi}Ly6C⁺CD106⁺SiglecF⁻CD115⁻CD205⁻CD11b^{Lo}Gr1^{Lo}CXCR4^{Hi}, the pre-neutrophils are CD101⁻cKit^{lo}Ly6C⁺CD106⁺⁺SiglecF⁻CD115⁻CD205⁺CD11b^{Hi}Gr1^{Hi} CXCR4^{Hi} or CD101⁻ cKit^{int}Ly6C⁺CD106⁺⁺SiglecF⁻CD115⁻CD205⁺CD11b^{Hi}Gr1^{Hi} CXCR4^{Hi} the immature neutrophils are CD101⁻ cKit^{int}Ly6C⁺CD106⁺SiglecF⁻CD115⁻CD205⁺CD11b^{Hi}Gr1^{Hi}CXCR4^{Lo} or CD101⁻ cKit^{lo}Ly6C⁺CD106⁺SiglecF⁻CD115⁻CD205⁺CD11b^{Hi}Gr1^{Hi}CXCR4^{Lo} and the mature neutrophils are CD101⁺cKit⁻Ly6C⁺CD106^{lo}SiglecF⁻CD115⁻CD205⁺CD11b^{Hi}Gr1^{Hi}CXCR4^{Lo}.

63. A kit for separating neutrophils, the kit comprising:
an agent for detecting the expression of CD101 on the neutrophils; and/or
a separator for separating a first population comprising proliferative neutrophils and a second population comprising mature neutrophils according to the expression of CD101 on the neutrophils.

64. The kit according to claim 63, wherein the first population expresses CD101⁻ and the second population expresses CD101⁺, the kit is for separating human neutrophils and the kit further comprises an agent for detecting the expression of CD10 on the human neutrophils, and the separator is adapted to separate the neutrophils according to the expression of CD10 on the neutrophils, wherein the first population comprising proliferative neutrophils are CD10⁻CD101⁻, and the second population comprising mature neutrophils are CD10⁺CD101⁺, optionally the second population further comprises immature neutrophils that are CD10⁻CD101⁺, optionally the agent for detecting the expression of CD10 is an antibody adapted to target CD10, and/or wherein the agent for detecting the expression of CD101 is an antibody adapted to target CD101.

65. The kit according to claim 63, wherein the kit further comprises an agent for detecting the expression on the neutrophils one or more biomarkers selected from a group consisting of CD49d, CD16, CXCR2, CD34, CD66, CD15, CD71, and CD11b, and wherein the separator is adapted to separate the neutrophils according to the expression of one or more of CD49d, CD16, CXCR2, CD34, CD66, CD15, CD71, and CD1lb on the neutrophils, optionally the kit is for separating murine neutrophils, and wherein the separator is further adapted to separate the neutrophils according to the expression of CD101 and/or cKit, wherein the first population comprising proliferative neutrophils is one of cKit^{hi}CD101⁻, cKit^{int}CD101⁻, or cKit^{lo}CD101⁻ and the second population comprising mature neutrophils are cKit-CD101⁺, optionally, wherein the first population further comprises immature neutrophils that are cKit^{lo}CD101⁺.

66. The kit according to claim 63, wherein the agent for detecting the expression of CD101 and/or cKit is an antibody adapted to target CD101 and/or cKit, the kit further comprises an agent for detecting the expression on the neutrophils of one or more biomarkers selected from a group consisting of CD101, cKit, Ly6C, CD106, SiglecF, CD115, CD205, CD11b, Gr1, and CXCR4, and wherein the separator is adapted to separate the neutrophils according to the expression of CD101, cKit, Ly6C, CD106, SiglecF, CD115, CD205, CD11b, Gr1, and/or CXCR4 on the neutrophils.

67. The method of claim 55, further comprising isolating and/or enriching a desired neutrophil, the method comprising:
categorizing neutrophils in a sample into a first population comprising proliferative neutrophils and a second population comprising mature neutrophils according to the expression of CD101 on the neutrophils; and isolating and/or enriching one or more neutrophil from the first population and/or the second population.

68. A method of treating immunodeficiency related diseases and/or disorders in a patient and/or enhancing the immune system of a patient, the method comprising administering a therapeutically effective amount of proliferative neutrophils to a patient, wherein the proliferative neutrophils are CD10⁻CD101⁻.

69. The method of claim 68, the method further comprising the steps of:
(a) obtaining a population of cells comprising neutrophils; and
(b) isolating proliferative neutrophils from the population of cells according to CD10 and/or CD101 expression on the neutrophils, wherein the proliferative neutrophils are CD10⁻CD101⁻.

70. The method of claim 68, wherein the method further comprising diagnosing or prognosing a medical condition in the patient, the method comprising the steps of:
(a) testing a sample comprising neutrophils obtained from the patient, to detect the expression of CD10 and/or CD101 on the neutrophils;
(b) measuring the levels of proliferative neutrophils, immature neutrophils and/or mature neutrophils in the sample, wherein proliferative neutrophils are CD10⁻CD101⁻, immature neutrophils are CD10⁻CD101⁺, and mature neutrophils are CD10⁺CD101⁺; and
(c) comparing the levels of the proliferative neutrophils, immature neutrophils and/or mature neutrophils in the sample, to reference levels in a control to determine the absence or presence of the medical condition, or to predict the course of the medical condition, optionally
the sample is a bone marrow sample and/or a spleen sample, and wherein a level of proliferative neutrophils in the sample higher than the reference level in the control indicates that the patient has an inflammatory medical condition, optionally
the inflammatory medical condition is associated with an autoimmune disease, sepsis and/or cancer.

71. The method according to claim 68, wherein a level of immature neutrophils in the sample higher than the reference level in the control indicates that the patient has the medical condition, optionally the level of immature neutrophils correlates with the progression of the medical condition.

72. The method according to claim 68, wherein the sample is a blood sample or a tumor sample, and wherein the medical condition is cancer, optionally the cancer is pancreatic cancer.

73. The kit of claim 63, wherein the kit is for detecting and/or predicting inflammation in a patient, the kit comprises:
an agent for detecting the expression of CD10 on neutrophils and/or an agent for detecting the expression of CD101 on neutrophils to measure the level of proliferative neutrophils in a sample taken from the patient, wherein the proliferative neutrophils are CD10⁻CD101⁻; and
a reference level for comparing the measured level of proliferative or immature neutrophils,
wherein a level of proliferative neutrophils in the sample higher than the reference level indicates that the patient has an inflammatory medical condition.

74. The kit of claim 63, wherein the kit is for diagnosis and/or prognosing cancer in a patient, the kit comprises:
an agent for detecting the expression of CD10 on neutrophils and/or an agent for detecting the expression of CD101 on neutrophils to measure the level of immature neutrophils in a sample taken from the patient, wherein the immature neutrophils are CD10⁻CD101⁺; and
a reference level for comparing the measured level of immature neutrophils,
wherein a level of immature neutrophils in the sample higher than the reference level indicates that the patient has cancer, and/or wherein the level of immature neutrophils correlates with the progression of cancer.
